(19) United States
(12) Patent Application Publication
(10) Pub. No.: US 2024/0199661 A1
(43) Pub. Date: Jun. 20, 2024

(54) BICYCLIC HETEROARYL PHOSPHONATE DERIVATIVES AS ECTONUCLEOTIDE PYROPHOSPHATASE PHOSPHODIESTERASE 1 INHIBITORS

(71) Applicant: RIBOSCIENCE LLC, Palo Alto, CA (US)

(72) Inventor: Ronald HAWLEY, Palo Alto, CA (US)

(21) Appl. No.: 18/551,850

(22) PCT Filed: Mar. 30, 2022
(86) PCT No.: PCT/US2022/022524

§ 371 Date: Sep. 21, 2023

Related U.S. Application Data
(60) Provisional application No. 63/168,548, filed on Mar. 31, 2021.

(51) Int. Cl. C07F 9/6512; A61K 31/675; C07F 9/6553

(52) U.S. Cl. CPC C07F 9/6512; A61K 31/675; C07F 9/655345

(57) ABSTRACT

The present disclosure provides certain bicyclic heteroaryl phosphonate compounds that inhibit ectonucleotide pyrophosphatase/phosphodiesterase 1 (ENPP1) enzymatic activity and are therefore useful for the treatment of diseases and conditions Also provided herein are pharmaceutical compositions containing such compounds and processes for preparing such compounds.

### CROSS-REFERENCES TO RELATED APPLICATIONS

This application claims the benefit of priority under 35 U.S.C. § 119(e) to U.S. Provisional Application Ser. No. 63/168,548 filed Mar. 31, 2021, the disclosure of which is incorporated herein by reference in its entirety for all purposes.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### FIELD OF THE DISCLOSURE

The present disclosure provides certain bicyclic heteroaryl phosphonate compounds that inhibit ectonucleotide pyrophosphatase/phosphodiesterase 1 (ENPP1) enzymatic activity and are therefore useful for the treatment of diseases treatable by inhibition of ENPP1. Also provided are pharmaceutical compositions containing such compounds and processes for preparing such compounds.

### BACKGROUND

ENPP1 enzyme is present in a wide range of tissues and cell types, such as lymphocytes, macrophages, liver, brain, heart, kidney, vascular smooth muscle cells, and chondrocytes. ENPP1 hydrolyzes ATP and other nucleoside triphosphates and releases AMP or other nucleoside monophosphates as well as pyrophosphate (PPi) (Kato K et al. 2012 PNAS 109:16876-16881; Hessle L et al. 2002 PNAS 99:9445-9449). The enzyme can also hydrolyze other nucleoside monophosphate esters (Kato K et al. 2012 PNAS 109:16876-16881). ENPP1 has been identified as the dominant 2′-3′-cGAMP hydrolase in cultured cells, tissue extracts and blood (Li L et al. 2014 Nat Chem Biol 10:1043-1048). Tissues and blood from ENPP1 knockout mice lack 2′-3′-cGAMP hydrolase activity. Elevated levels of ENPP1 have been associated with calcific aortic valve disease (CAVD) and calcium pyrophosphate dihydrate (CPPD) disease, an inflammatory disease resulting from CPPD crystal deposits in the joint and surrounding tissues (Cote N et al. 2012 Eur J Pharmacol 689:139-146; Johnson K et al. 2001 Arthritis Rheum 44:1071). ENPP1 expression is upregulated in certain hepatocellular carcinomas, glioblastomas, melanomas, testicular, pancreatic and thyroid and breast cancers and has been associated with resistance to chemotherapy (see Lau W M et al. 2013 PLoS One 8:5; Bageritz J et al. 2014 Mol Cell Oncology 1:3; Bageritz J et al. 2014 Cell Death, Differentiation 21:929-940; Umar A et al. 2009 Mol Cell Proteomics 8:1278-1294). ENPP1 upregulation and variants of ENPP1 are also associated with insulin resistance and type 2 diabetes (Meyre D et al. 2005 Nat Genet 37:863-867; Maddux B A et al. 1995 Nature 373:448-451; Rey D et al. 2012 Mol Biol Rep 39:7687-7693) and enzyme activity of ENPP1 was reported to be required for the inhibition of insulin receptor signaling (Chin C N et al. 2009 Eur J Pharmacol 606:17-24).

Cyclic GMP-AMP synthase (cGAS) is a pattern recognition receptor that synthesizes the endogenous messenger molecule cGAMP from ATP and GTP in response to the presence of DNA derived from viruses, bacteria, damaged mitochondria or cancer cells. The cGAMP molecule then binds to the stimulator of interferon genes (STING) protein, which initiates a signaling response that activates innate immunity and results in the production of type I interferon, antiviral and immune-stimulatory cytokines (Sun L et al. 2013 Science 339:786-791; Wu J et al. 2013 Science 339:826-830; Gao D et al. 2013 Science 341:903-906; Li X et al. 2013 Science 341:1390-1394; Schoggins J W et al. 2014 Nature 505:691-695; Wassermann R et al. 2015 Cell Host Microbe 17:799-810; Watson R O et al. 2015 Cell Host Microbe 17:811-819; Collins A et al. 2015 Cell Host Microbe 17:820-828; West A et al. 2015 Nature 520:533-557; Woo S R et al. 2014 Immunity 41:830-842; Deng L et al. 2014 Immunity 41:843-852; Chen Q et al. 2016 Nat Immunol 17:1142-1148). The cGAS enzyme, cGAMP messenger and STING are is also involved in host defense against RNA viruses and the immune control of tumor development (Aguirre S et al. 2012 PLoS Pathog 8: e1002934; Barber G N 2015 Nat Rev Immunol 15:760-770). ENPP1 has been identified as the enzyme that naturally hydrolyzes cGAMP and therefore counteracts the innate immune response against infectious agents, damaged cells and cancer cells (Li L et al. 2014 Nat Chem Biol 10:1043-1048). The efficacy of non-hydrolyzable cGAMP analogs in inducing functional immune responses is higher than that of natural, hydrolysable cGAMP (Li L et al. 2014 Nat Chem Biol 10:1043-1048; Corrales L et al. 2015 Cell Rep 11:1018-1030). Virus infection has been demonstrated to be facilitated by ENPP1 overexpression and is attenuated by silencing of ENPP1 (Wang J et al. 2018 Mol Immunol 95:56-63).

Inhibitors of cGAMP hydrolysis may therefore be used to increase the effectiveness of immune responses against cancer cells and tumors and against infections by RNA or DNA viruses or bacteria. Inhibitors of ENPP1 and of cGAMP or nucleoside triphosphate hydrolysis may also be used for the treatment of inflammatory diseases that are associated with elevated nucleotidase levels, reduced nucleoside triphosphate, reduced cGAMP or reduced nucleoside monophosphate ester levels or diseases associated with elevated nucleoside or nucleoside monophosphate levels. For these reasons, ENPP1 is an attractive therapeutic target for the treatment of diseases, including cancer.

The present disclosure addresses these needs and provides related advantages as well.

### SUMMARY

In a first aspect, provided is a compound of Formula (I):

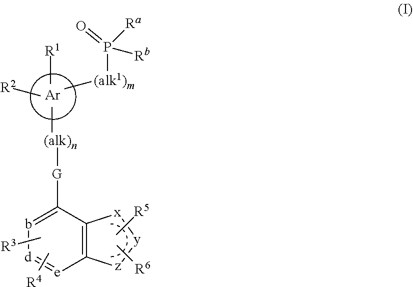

wherein:
[symbol] dashed line is a bond between x and y or y and z;
b, d, and e are CH; or one or two of b, d, and e are N and remaining of b, d, and e are CH;
one of x and z is NH, O, or S and the other of x and z is CH or N; and y is CH or N; provided that, at least one of y and the x or z that is CH or N, is CH;
G is a bond, NR (where R is hydrogen or alkyl), O, S, or SO₂;
alk is alkylene optionally substituted with one, two, or three halo or alkynylene, provided that when alk is alkynylene G is a bond and n is 1;
alk¹ is alkylene optionally substituted with one, two, or three halo;
m and n are independently 0 or 1; provided that at least one of m and n is 1;
Ar is aryl, heteroaryl, or heterocyclyl;
R^{a} and R^{b} are independently selected from hydroxy, alkoxy, —Oaryl (where aryl is optionally substituted with one to three substituents independently selected from alkyl, alkenyl, alkoxy, halo, haloalkyl, amino, alkylamino, dialkylamino, cyano, and nitro), —O—(CH₂)OCOR^{c} (where R^{c} is alkyl), —O—(CH₂)OCOOR^{c1} (where R^{c1} is alkyl), —O-(alk²)OR^{d} (where alk² is alkylene and R^{d} is alkyl), —S—(CH₂)₂SCOR^{c} (where R^{c} is alkyl), and —NR^{g}—(CHR^{h})OCOR^{f} (where R^{h} is hydrogen, alkyl, hydroxymethyl, thiomethyl, methylthiomethyl, amidinopropyl, indol-3-ylmethyl, indol-4-ylmethyl, carboxymethyl, carboxyethyl, aminocarbonylmethyl, aminocarbonylethyl, phenyl or phenylalkyl (wherein phenyl, either alone or as part of phenylalkyl, is optionally substituted with one, two, or three substituents independently selected from alkyl, alkoxy, halo, hydroxy, cyano, and nitro), R^{f} is alkyl or benzyl and R^{g} is hydrogen or R^{g} together with R^{h} forms —(CH₂)₃—); or
R^{a} and R^{b} together with the phosphorus atom to which they are attached form a ring of formula (a):

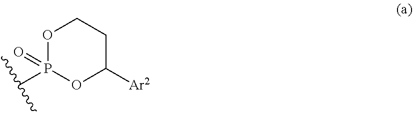

wherein Ar² is phenyl or six membered heteroaryl wherein the phenyl and six membered heteroaryl are optionally substituted with one to three halo;
R¹ and R² are independently absent, alkyl, alkoxy, halo, haloalkyl, haloalkoxy, or cyano;
R³ and R⁴ are independently absent, alkyl, alkoxy, hydroxy, halo, haloalkyl, haloalkoxy, alkylsulfonyl, hydroxyalkyl, alkoxyalkyl, hydroxyalkoxy, alkoxyalkoxy, hydroxyalkylamino, alkoxyalkylamino, amino, aminoalkyl, aminoalkoxy, aminoalkylamino, heterocyclyl, heterocyclyloxy, heterocyclylamino (wherein heterocyclyl, either alone or part of heterocyclyloxy and heterocyclylamino, is optionally substituted with Rⁱ, R^{j}, and/or R^{k} independently selected from alkyl, halo, hydroxy, alkoxy, hydroxyalkyl, alkoxyalkyl, and aminoalkyl), heterocyclylalkyl, heterocyclylalkyloxy, heterocyclylalkylamino (wherein the heterocyclyl ring in heterocyclylalkyl, heterocyclylalkyloxy, and heterocyclylalkylamino is optionally substituted with one, two, or three substituents independently selected from alkyl, halo, hydroxy, alkoxy, hydroxyalkyl, alkoxyalkyl, and aminoalkyl), cycloalkyloxy, phenyl, heteroaryl, phenylalkenyl, heteroarylalkenyl, phenyloxy, or heteroaryloxy (where phenyl, by itself or as part of phenylalkenyl and phenyloxy and heteroaryl, by itself or as part of heteroarylalkenyl and heteroaryloxy are optionally substituted with one, two, or three substituents independently selected from alkyl, hydroxy, alkoxy, halo, haloalkyl, haloalkoxy, and cyano); provided that when two of b, d, and e are N, then at least one of R³ and R⁴ is absent;
R⁵ is absent, alkyl, hydroxy, alkoxy, alkoxycarbonyl, halo, acylamino, hydroxyalkylamino, alkoxyalkylamino, cyano, aminocarbonyl, alkylaminocarbonyl, dialkylaminocarbonyl, aminoalkyloxycarbonyl, alkoxyalkyloxycarbonyl, or hydroxyalkylaminocarbonyl; and
R⁶ is absent or alkyl; provided that one of R³ and R⁶ is absent when two of x, y, and z are other than CH; or
a pharmaceutically acceptable salt thereof.

It is understood in Formula (I) and all of the subembodiments described herein that when one or more of R³, R⁴, R⁵, and R⁶ are present, these variable groups replace a hydrogen atom at the ring vertex to which each is attached. Additionally, in Formula (I) and all of the relevant subembodiments described herein, it is understood that the dashed line in the fused five membered ring comprising ring vertices x, y, and z indicates the presence of one double bond between either x and y or y and z.

In a second aspect, provided is a compound of Formula (IA):

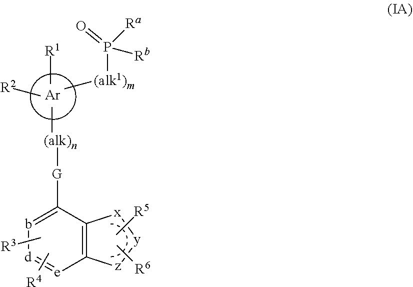

wherein:
[symbol] dashed line is a bond between x and y or y and z;
b, d, and e are CH; or one or two of b, d, and e are N and remaining of b, d, and e are CH;
x is NH, O, or S;
y and z are independently CH or N; provided that at least one of y and z is CH;
G is NR (where R is hydrogen or alkyl), O, or S;
alk is alkylene optionally substituted with one, two, or three halo;
alk¹ is alkylene optionally substituted with one, two, or three halo;
m and n are independently 0 or 1; provided that at least one of m and n is 1;
Ar is aryl, heteroaryl, or heterocyclyl;
R^{a} and R^{b} are independently selected from hydroxy, alkoxy, —Oaryl (where aryl is optionally substituted with one to three substituents independently selected from alkyl, alkenyl, alkoxy, halo, haloalkyl, amino, alkylamino, dialkylamino, cyano, and nitro), —O—(CH₂)OCOR^{c} (where R^{c} is alkyl), —O—(CH₂)OCOOR^{c1} (where R^{c1} is alkyl), —O-(alk²)OR^{d} (where alk² is alkylene and R^{d} is alkyl), —S—(CH₂)₂SCOR^{e} (where R^{c} is alkyl), and —NR^{g}—(CHR^{h})OCOR^{f} (where R^{h} is hydrogen, alkyl, hydroxymethyl, thiomethyl, methylthiomethyl, amidinopropyl, indol-3-ylmethyl, indol-4-ylmethyl, carboxymethyl, carboxyethyl, aminocarbonylmethyl, aminocarbonylethyl, phenyl or phenylalkyl (wherein phenyl, either alone or as part of phenylalkyl, is optionally substituted with one, two, or three substituents independently selected from alkyl, alkoxy, halo, hydroxy, cyano, and nitro), R^{f} is alkyl or benzyl and R^{g} is hydrogen or R^{g} together with R^{h} forms —(CH₂)₃—); or
R^{a} and R^{b} together with the phosphorus atom to which they are attached form a ring of formula (a):

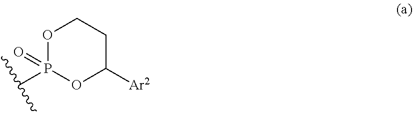

wherein Ar² is phenyl or six membered heteroaryl wherein the phenyl and six membered heteroaryl are optionally substituted with one to three halo;
R¹ and R² are independently absent, alkyl, alkoxy, halo, haloalkyl, haloalkoxy, or cyano;
R³ and R⁴ are independently absent, alkyl, alkoxy, hydroxy, halo, haloalkyl, haloalkoxy, alkylsulfonyl, hydroxyalkyl, alkoxyalkyl, hydroxyalkoxy, alkoxyalkoxy, hydroxyalkylamino, alkoxyalkylamino, amino, aminoalkyl, aminoalkoxy, aminoalkylamino, heterocyclyl, heterocyclyloxy, heterocyclylamino (wherein heterocyclyl, either alone or part of heterocyclyloxy and heterocyclylamino, is optionally substituted with Rⁱ, R^{j}, and/or R^{h} independently selected from alkyl, halo, hydroxy, alkoxy, hydroxyalkyl, alkoxyalkyl, and aminoalkyl), heterocyclylalkyl, heterocyclylalkyloxy, heterocyclylalkylamino (wherein the heterocyclyl ring in heterocyclylalkyl, heterocyclylalkyloxy, and heterocyclylalkylamino is optionally substituted with one, two, or three substituents independently selected from alkyl, halo, hydroxy, alkoxy, hydroxyalkyl, alkoxyalkyl, and aminoalkyl), cycloalkyloxy, phenyloxy, or heteroaryloxy (where phenyl in phenyloxy and heteroaryl in heteroaryloxy are optionally substituted with one, two, or three substituents independently selected from alkyl, hydroxy, alkoxy, halo, haloalkyl, haloalkoxy, and cyano); provided that when two of b, d, and e are N, then at least one of R³ and R⁴ is absent;
R⁵ is absent, alkyl, hydroxy, halo, acylamino, hydroxyalkylamino, alkoxyalkylamino, cyano, aminocarbonyl, alkylaminocarbonyl, or dialkylaminocarbonyl; and
R⁶ is absent or alkyl; provided that one of R³ and R⁶ is absent when two of x, y, and z are other than CH; or
a pharmaceutically acceptable salt thereof.

In a third aspect, provided is a compound of Formula (IB):

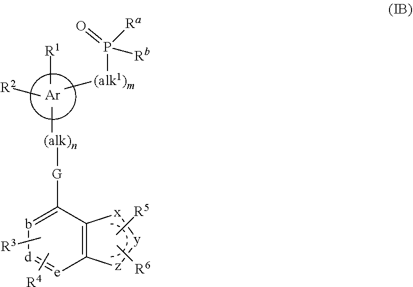

wherein:
[symbol] dashed line is a bond between x and y or y and z;
b, d, and e are CH; or one or two of b, d, and e are N and remaining of b, d, and e are CH;
x is NH, O, or S;
y and z are independently CH or N; provided that at least one of y and z is CH;
G is NR (where R is hydrogen or alkyl) or O;
alk is alkylene optionally substituted with one, two, or three halo;
alk¹ is alkylene optionally substituted with one, two, or three halo;
m and n are independently 0 or 1; provided that at least one of m and n is 1;
Ar is aryl, heteroaryl, or heterocyclyl;
R^{a} and R^{b} are independently selected from hydroxy, alkoxy, —Oaryl (where aryl is optionally substituted with one to three substituents independently selected from alkyl, alkenyl, alkoxy, halo, haloalkyl, amino, alkylamino, dialkylamino, cyano, and nitro), —O—(CH₂)OCOR^{c} (where R^{c} is alkyl), —O—(CH₂)OCOOR^{c1} (where R^{c1} is alkyl), —O-(alk²)OR^{d} (where alk² is alkylene and R^{d} is alkyl), —S—(CH₂)₂SCOR^{e} (where R^{c} is alkyl), and —NR^{g}—(CHR^{h})OCOR^{f} (where R^{h} is hydrogen, alkyl, hydroxymethyl, thiomethyl, methylthiomethyl, amidinopropyl, indol-3-ylmethyl, indol-4-ylmethyl, carboxymethyl, carboxyethyl, aminocarbonylmethyl, aminocarbonylethyl, phenyl or phenylalkyl (wherein phenyl, either alone or as part of phenylalkyl, is optionally substituted with one, two, or three substituents independently selected from alkyl, alkoxy, halo, hydroxy, cyano and nitro), R^{f} is alkyl or benzyl and R^{g} is hydrogen or R^{g} together with R^{h} forms —(CH₂)₃—); or
R^{a} and R^{b} together with the phosphorus atom to which they are attached form a ring of formula (a):

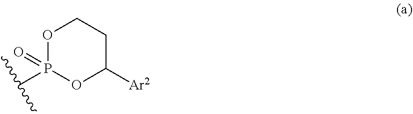

wherein Ar² is phenyl or six membered heteroaryl optionally substituted with one to three halo;
R¹ and R² are independently absent, alkyl, alkoxy, halo, haloalkyl, haloalkoxy, or cyano;
R³ and R⁴ are independently absent, alkyl, alkoxy, hydroxy, halo, haloalkyl, haloalkoxy, alkylsulfonyl, hydroxyalkyl, alkoxyalkyl, hydroxyalkoxy, alkoxyalkoxy, hydroxyalkylamino, alkoxyalkylamino, amino, aminoalkyl, aminoalkoxy, aminoalkylamino, heterocyclyl, heterocyclyloxy, heterocyclylamino (wherein heterocyclyl, either alone or part of heterocyclyloxy, and heterocyclylamino is optionally substituted with Rⁱ, R^{j}, and/or R^{k} independently selected from alkyl, halo, hydroxy, alkoxy, hydroxyalkyl, alkoxyalkyl, and aminoalkyl), heterocyclylalkyl, heterocyclylalkyloxy, heterocyclylalkylamino (wherein the heterocyclyl ring in heterocyclylalkyl, heterocyclylalkyloxy, and heterocyclylalkylamino is optionally substituted with one, two, or three substituents independently selected from alkyl, halo, hydroxy, alkoxy, hydroxyalkyl, alkoxyalkyl, and aminoalkyl), cycloalkyloxy, phenyloxy, or heteroaryloxy (where phenyl in phenyloxy and heteroaryl in heteroaryloxy are optionally substituted with one, two, or three substituents independently selected from alkyl, hydroxy, alkoxy, halo, haloalkyl, haloalkoxy, and cyano); provided that when two of b, d, and e are N, then at least one of R³ and R⁴ is absent;
R⁵ is absent, alkyl, hydroxy, halo, acylamino, hydroxyalkylamino, alkoxyalkylamino, cyano, aminocarbonyl, alkylaminocarbonyl, or dialkylaminocarbonyl; and
R⁶ is absent or alkyl; provided that one of R³ and R⁶ is absent when two of x, y, and z are other than CH; or
a pharmaceutically acceptable salt thereof.

In a fourth aspect, provided is a pharmaceutical composition comprising a compound of Formula (I), (IA), or (IB) (or any of the embodiments thereof described herein) or a pharmaceutically acceptable salt thereof and a pharmaceutically acceptable excipient.

In a fifth aspect, provided are methods of treating a disease or mediated by ENPP1 in a patient, preferably in a patient recognized as needing such a treatment, comprising administering to the patient (i) a compound of Formula (I), (IA), or (IB) (or any of the embodiments thereof described herein) or a pharmaceutically acceptable salt thereof in a therapeutically effective amount or (ii) a pharmaceutical composition comprising a compound of Formula (I), (IA), or (IB) (or any of the embodiments thereof described herein) or a pharmaceutically acceptable salt thereof and a pharmaceutically acceptable excipient in a therapeutically effective amount. In one embodiment, the disease is cancer such as hepatocellular carcinomas, glioblastomas, melanomas, testicular, pancreatic, thyroid and breast cancer. In another embodiment, the disease is an inflammatory disease e.g., calcific aortic valve disease and calcium pyrophosphate dihydrate. In yet another embodiment the disease metabolic disease e.g., type 2 diabetes or a viral infection.

In a sixth aspect, provided is a compound of Formula (I), (IA), or (IB) (or any embodiments thereof described herein) or a pharmaceutically acceptable salt thereof for use as a medicament. In one embodiment, the medicament is for use in the treatment of cancer such as hepatocellular carcinomas, glioblastomas, melanomas, testicular, pancreatic, thyroid and breast cancer. In another embodiment, the medicament is for use in the treatment of an inflammatory disease e.g., calcific aortic valve disease and calcium pyrophosphate dihydrate. In yet another embodiment, the medicament is for use in the treatment of a metabolic disease e.g., type 2 diabetes or a viral infection.

In a seventh aspect provided is a compound of Formula (I), (IA), or (IB) or a pharmaceutically acceptable salt thereof (and any embodiments thereof disclosed herein) for use in treating a disease in a patient in which the activity of ENPP1 contributes to the pathology and/or symptoms of the disease. In one embodiment, the disease is cancer such as hepatocellular carcinomas, glioblastomas, melanomas, testicular, pancreatic, thyroid and breast cancer. In another embodiment, the disease is an inflammatory disease e.g., calcific aortic valve disease and calcium pyrophosphate dihydrate. In yet another embodiment, the disease metabolic disease e.g., type 2 diabetes or a viral disease.

Unless noted otherwise, reference to an embodiment above includes subembodiments contained within such embodiment.

In any of the aforementioned aspects involving the treatment of cancer, are further embodiments comprising administering the compound of Formula (I), (IA), or (IB) or a pharmaceutically acceptable salt thereof (or any embodiments thereof disclosed herein) in combination with at least one additional anticancer. When combination therapy is used, the agents can be administered simultaneously or sequentially.

### DETAILED DESCRIPTION

### Definitions

Unless otherwise stated, the following terms used in the specification and claims are defined for the purposes of this Application and have the following meaning:

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl, pentyl, and the like.

“Alkylene” means a linear saturated divalent hydrocarbon radical of one to six carbon atoms or a branched saturated divalent hydrocarbon radical of three to six carbon atoms unless otherwise stated e.g., methylene, ethylene, propylene, 1-methylpropylene, 2-methylpropylene, butylene, pentylene, and the like.

“Alkenyl” means a linear or branched monovalent hydrocarbon radical of two to six carbon atoms containing a double bond, e.g., ethenyl, propenyl, 2-propenyl, and the like.

“Alkenylene” means a linear or branched divalent hydrocarbon radical of two to six carbon atoms containing a double bond, e.g., ethenylene, propenylene, and the like.

“Alkynylene” means a linear or branched divalent hydrocarbon radical of two to six carbon atoms containing a triple bond, e.g., ethynylene, propynylene, and the like.

“Alkylsulfonyl” means —SO₂R radical where R is alkyl as defined above, e.g., methylsulfonyl, ethylsulfonyl, and the like.

“Amino” means a —NH₂.

“Aminocarbonyl” means —CONH₂.

“Aminocarbonylmethyl” means —CH₂CONH₂.

“Aminocarbonylethyl” means —(CH₂)₂CONH₂.

“Alkylaminocarbonyl” means —CONHR radical where R is alkyl as defined above, e.g., methylaminocarbonyl, ethylaminocarbonyl, and the like.

“Acylamino” means —NHCOR radical where R is hydrogen (—NHCOR is formyl), alkyl, phenyl, or heterocyclyl as defined above, e.g., acetylamino, ethylcarbonylamino, benzoylamino, azetidin-1-ylcarbonylamino, and the like.

“Alkylamino” means a —NHR radical where R is alkyl as defined above, e.g., methylamino, ethylamino, propylamino, or 2-propylamino, and the like.

“Aminoalkyl” means a linear monovalent hydrocarbon radical of one to six carbon atoms or a branched monovalent hydrocarbon radical of three to six carbons substituted with —NR′R″ where R′ and R″ are independently hydrogen or alkyl as defined above, e.g., aminomethyl, aminoethyl, methylaminomethyl, dimethylaminomethyl, and the like.

“Aminoalkylamino” means a —NR^{a}R^{b} radical where R^{a} is hydrogen or alkyl and R^{b} is aminoalkyl as defined above, e.g., aminoethylamino, dimethylaminoethylamino, diethylaminoethylamino, dimethylaminopropylamino, diethylaminopropylamino, and the like.

“Aminoalkyloxy” or “aminoalkoxy” means a —OR^{a} radical where R is aminoalkyl as defined above, e.g., aminoethyloxy, dimethylaminoethyloxy, diethylaminoethyloxy, dimethylaminopropyloxy, diethylaminopropyloxy, and the like.

“Aminoalkyloxycarbonyl” means a —COOR radical where R is aminoalkyl as defined above, e.g., aminoethyloxycarbonyl, dimethylaminomethyloxycarbonyl, and the like.

“Alkoxy” means a —OR radical where R is alkyl as defined above, e.g., methoxy, ethoxy, propoxy, or 2-propoxy, n-, iso-, or tert-butoxy, and the like.

“Alkoxyalkyl” means a linear monovalent hydrocarbon radical of one to six carbon atoms or a branched monovalent hydrocarbon radical of three to six carbons substituted with at least one alkoxy group, such as one or two alkoxy groups, as defined above, e.g., 2-methoxyethyl, 1-, 2-, or 3-methoxypropyl, 2-ethoxyethyl, and the like.

“Alkoxyalkyloxycarbonyl” means a —COOR radical where R is alkoxyalkyl as defined above, e.g., methoxyethyloxycarbonyl, methoxymethyloxycarbonyl, and the like.

“Alkoxycarbonyl” means a —COOR radical where R is alkyl as defined above, e.g., methoxycarbonyl, ethoxycarbonyl, propoxycarbonyl, or 2-propoxycarbonyl, n-, iso-, or tert-butoxycarbonyl, and the like.

“Alkoxyalkylamino” means a —NRR′ radical where R is hydrogen or alkyl and R′ is alkoxyalkyl as defined above, e.g., methoxyethylamino, ethoxyethylamino, propoxypropylamino, ethoxypropylamino, and the like.

“Alkoxyalkyloxy” or “alkoxyalkoxy” means a —(O)R radical where R is alkoxyalkyl as defined above, e.g., methoxyethoxy, ethoxyethoxy, and the like.

“Amidinopropyl” refers to —(CH₂)₃NHC(═NH)NH₂ radical.

“Aryl” means a monovalent monocyclic or bicyclic aromatic hydrocarbon radical of 6 to 10 ring atoms e.g., phenyl or naphthyl.

“Cycloalkyl” means a cyclic saturated monovalent hydrocarbon radical of three to ten carbon atoms, e.g., cyclopropyl, cyclobutyl, cyclopentyl, or cyclohexyl, and the like.

“Cycloalkyloxy” means a —OR radical where R is cycloalkyl (including specific cycloalkyl rings) as defined above e.g., cyclopropyloxy, and the like.

“Carboxy” means —COOH.

“Carboxymethyl” means —CH₂COOH.

“Carboxyethyl” means —(CH₂)₂COOH.

“Dialkylaminocarbonyl” means —CONHRR′ where R and R′ are independently alkyl as defined above, e.g., dimethylaminocarbonyl, methylethylaminocarbonyl, and the like.

“Dialkylamino” means a —NRR′ radical where R and R′ are alkyl as defined above, e.g., dimethylamino, methylethylamino, and the like.

“Halo” means fluoro, chloro, bromo, or iodo, preferably fluoro or chloro.

“Haloalkyl” means alkyl radical as defined above, which is substituted with one or more halogen atoms, such as one to five halogen atoms, such as fluorine or chlorine, including those substituted with different halogens, e.g., —CH₂Cl, —CF₃, —CHF₂, —CH₂CF₃, —CF₂CF₃, —CF(CH₃)₂, and the like. When the alkyl is substituted with only fluoro, it can be referred to in this Application as fluoroalkyl.

“Haloalkoxy” means a —OR radical where R is haloalkyl as defined above e.g., —OCF₃, —OCHF₂, and the like. When R is haloalkyl where the alkyl is substituted with only fluoro, it is referred to in this Application as fluoroalkoxy.

“Hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxy-ethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl. When hydroxyalkyl is —CH₂OH it is referred to herein as hydroxymethyl.

“Hydroxyalkylamino” means a —NR^{a}R^{b} radical where R^{a} is hydrogen or alkyl and R^{b} is hydroxyalkyl as defined above, e.g., hydroxyethylamino, hydroxypropylamino, and the like.

“Hydroxyalkylaminocarbonyl” means a —CONR^{a}R^{b} radical where R is hydrogen or alkyl and R^{b} is hydroxyalkyl as defined above, e.g., hydroxyethylaminocarbonyl, hydroxypropylaminocarbonyl, and the like.

“Hydroxyalkyloxy” or “hydroxyalkoxy” means a —OR^{a} radical where R^{a} is hydroxyalkyl as defined above, e.g., hydroxyethyloxy, hydroxypropyloxy, and the like.

“Heterocyclyl” means a saturated or unsaturated monovalent monocyclic group of 4 to 8 ring atoms in which one or two ring atoms are heteroatom selected from N, O, and S(O)ₙ, where n is an integer from 0 to 2, the remaining ring atoms being C. Additionally, one or two ring carbon atoms in the heterocyclyl ring can optionally be replaced by a —CO— group. More specifically the term heterocyclyl includes, but is not limited to, pyrrolidinyl, piperidinyl, homopiperidinyl, 2-oxopyrrolidinyl, 2-oxopiperidinyl, morpholinyl, piperazinyl, tetrahydro-pyranyl, thiomorpholinyl, and the like. When the heterocyclyl ring is unsaturated it can contain one or two ring double bonds provided that the ring is not aromatic. When the heterocyclyl group contains at least one nitrogen atom, it is also referred to herein as heterocycloamino and is a subset of the heterocyclyl group.

“Heterocyclylalkyl” or “heterocycloalkyl” means a -(alkylene)-R radical where R is heterocyclyl ring (including specific heterocyclyl rings) as defined above e.g., tetraydrofuranylmethyl, piperazinylmethyl, morpholinylethyl, and the like.

“Heterocyclylamino” means a —NRR′ radical where R is hydrogen or alkyl and R′ is heterocyclyl (including specific heterocyclyl rings) as defined above.

“Heterocyclylalkylamino” or “heterocycloalkylamino” means a —NRR′ radical where R is hydrogen or alkyl and R′ is heterocyclylalkyl ring (including specific heterocyclyl rings) as defined above e.g., tetraydrofuranylmethylamino, piperazinylethylamino, morpholinylethylamino, piperidinylmethylamino, and the like.

“Heterocyclyloxy” means a —OR radical where R is heterocyclyl (including specific heterocyclyl rings) as defined above e.g., piperidinyloxy, pyrrolidinyloxy, and the like.

“Heterocyclylalkyloxy” or “heterocycloalkyloxy” means a —OR radical where R is heterocyclylalkyl ring (including specific heterocyclyl rings) as defined above e.g., tetraydrofuranylmethyloxy, piperazinylethyloxy, morpholinylethyloxy, piperidinylmethyloxy, and the like.

“Heteroaryl” means a monovalent monocyclic or fused bicyclic aromatic radical of 5 to 10 ring atoms, unless otherwise stated, where one or more, (in one embodiment, one, two, or three), ring atoms are heteroatom selected from N. O, or S, the remaining ring atoms being carbon. Representative examples include, but are not limited to, pyrrolyl, thienyl, thiazolyl, imidazolyl, furanyl, indolyl, isoindolyl, oxazolyl, isoxazolyl, benzothiazolyl, benzoxazolyl, quinolinyl, isoquinolinyl, pyridinyl, pyrimidinyl, pyrazinyl, pyridazinyl, triazolyl, tetrazolyl, and the like. As defined herein, the terms “heteroaryl” and “aryl” are mutually exclusive. When the heteroaryl ring contains 5- or 6 ring atoms it is also referred to herein as 5- or 6-membered heteroaryl.

“Heteroaryloxy” means a —OR radical where R is heteroaryl (including specific heteroaryl rings) as defined above.

“Heteroarylalkenyl” means -(alkenylene)-R radical where R is heteroaryl and alkenylene are as defined above, e.g., 2-pyridinylethenylene, and the like.

“Methylthiomethyl” refers to —CH₂SCH₃ radical.

“Phenyloxy” means a —OR radical where R is phenyl.

“Phenylalkyl” means -(alkylene)-R radical where R is phenyl and alkylene is as defined above, e.g., benzyl, phenethyl, and the like.

“Phenylalkenyl” means -(alkenylene)-R radical where R is phenyl and alkenylene is as defined above, e.g., 2-phenylethenylene, and the like.

The present disclosure also includes protected derivatives of compounds of the present disclosure (I). For example, when compounds of the present disclosure contain groups such as hydroxy, carboxy, thiol or any group containing a nitrogen atom(s), these groups can be protected with a suitable protecting groups. A comprehensive list of suitable protective groups can be found in T. W. Greene, Protective Groups in Organic Synthesis, John Wiley & Sons, Inc. (1999), the disclosure of which is incorporated herein by reference in its entirety. The protected derivatives of compounds of the present disclosure can be prepared by methods well known in the art.

The present disclosure also includes polymorphic forms and deuterated forms of the compound of the present disclosure and/or a pharmaceutically acceptable salt thereof.

A “pharmaceutically acceptable salt” of a compound means a salt that is pharmaceutically acceptable and that possesses the desired pharmacological activity of the parent compound. Such salts include: acid addition salts, formed with inorganic acids such as hydrochloric acid, hydrobromic acid, sulfuric acid, nitric acid, phosphoric acid, and the like; or formed with organic acids such as formic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. It is understood that the pharmaceutically acceptable salts are non-toxic. Additional information on suitable pharmaceutically acceptable salts can be found in Remington's Pharmaceutical Sciences, 17th ed., Mack Publishing Company, Easton, PA, 1985, which is incorporated herein by reference in its entirety.

The compounds of the present disclosure may have asymmetric centers. Compounds of the present disclosure containing an asymmetrically substituted atom may be isolated in optically active or racemic forms. It is well known in the art how to prepare optically active forms, such as by resolution of materials. All chiral, diastereomeric, all mixtures of chiral or diasteromeric forms, and racemic forms are within the scope of this disclosure, unless the specific stereochemistry or isomeric form is specifically indicated. It will also be understood by a person of ordinary skill in the art that when a compound is denoted as (R) stereoisomer, it may contain the corresponding (S) stereoisomer as an impurity i.e., the (S) stereoisomer in less than about 5%, preferably 2% by wt and then it is denoted as a mixture of R and S isomers, the amounts of R or S isomer in the mixture is greater than about 5%, preferably 2% w/w.

Certain compounds of the present disclosure can exist as tautomers and/or geometric isomers. All possible tautomers and cis and trans isomers, as individual forms and mixtures thereof are within the scope of this disclosure.

Additionally, as used herein the term alkyl includes all the possible isomeric forms of said alkyl group. Furthermore, when the cyclic groups such as aryl, heteroaryl, heterocyclyl are substituted, they include all the positional isomers. Furthermore, all hydrates of a compound of the present disclosure are within the scope of this disclosure.

Certain structures provided herein are drawn with one or more floating substituents e.g., in the structure:

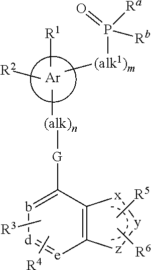

of Formula (I), (IA), and (IB), R¹, R², R³, R⁴, R⁵, and R⁶ are floating substituents. Unless provided otherwise or otherwise clear from the context, the floating substituent(s) may be present on any atom of the ring through which the substituent is drawn, where chemically feasible and valency rules permitting. For example, in the structure:

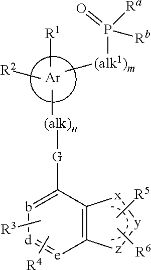

of Formula (I), (IB), and (IC), the R³ substituent can replace any hydrogen on the six membered aromatic ring portion of the bicyclic ring system when any of b, d, and e is CH.

The compounds of the present disclosure may also contain unnatural amounts of isotopes at one or more of the atoms that constitute such compounds. Unnatural amounts of an isotope may be defined as ranging from the amount found in nature to an amount 100% of the atom in question. that differ only in the presence of one or more isotopically enriched atoms. Exemplary isotopes that can be incorporated into compounds of the present invention, such as a compound of Formula (I) (and any embodiment thereof disclosed herein including specific compounds) include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I, and ¹²⁵I, respectively. Isotopically-labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, in compounds disclosed herein, including in Table 1 below one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁵F are useful for positron emission tomography (PET) studies to examine substrate receptor occupancy. Isotopically labeled compounds can generally be prepared by following procedures analogous to those disclosed in the Schemes or in the Examples herein, by substituting an isotopically labeled reagent for a non-isotopically labeled reagent.

“Oxo” or “carbonyl” means ═(O) group.

“Optional” or “optionally” means that the subsequently described event or circumstance may but need not occur, and that the description includes instances where the event or circumstance occurs and instances in which it does not. For example, “heterocyclyl group optionally substituted with an alkyl group” means that the alkyl may but need not be present, and the description includes situations where the heterocyclyl group is substituted with an alkyl group and situations where the heterocyclyl group is not substituted with alkyl.

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass ±10%, preferably ±5%, the recited value and the range is included.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “patient” is generally synonymous with the term “subject” and includes all mammals including humans. Examples of patients include humans, livestock such as cows, goats, sheep, pigs, and rabbits, and companion animals such as dogs, cats, and horses. Preferably, the patient is a human.

The terms “inhibiting” and “reducing,” or any variation of these terms in relation of EPPI, includes any measurable decrease or complete inhibition to achieve a desired result. For example, there may be a decrease of about, at most about, or at least about 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, reduction of EPP1 activity compared to normal.

“Treating” or “treatment” of a disease includes:
(1) preventing the disease, i.e. causing the clinical symptoms of the disease not to develop in a mammal that may be exposed to or predisposed to the disease but does not yet experience or display symptoms of the disease;
(2) inhibiting the disease, i.e., arresting or reducing the development of the disease or its clinical symptoms; or
(3) relieving the disease, i.e., causing regression of the disease or its clinical symptoms.

In one embodiment, treating or treatment includes inhibiting or relieving the disease.

A “therapeutically effective amount” means the amount of a compound of the present disclosure and/or a pharmaceutically acceptable salt thereof that, when administered to a patient for treating a disease, is sufficient to effect such treatment for the disease. The “therapeutically effective amount” will vary depending on the compound, the disease and its severity and the age, weight, etc., of the mammal to be treated.

“Thiomethyl” refers to —CH₂SH radical.

Representative compound of Formula (I) are disclosed in Table 1 below:

| Cpd # | Structure | Name |
|---|---|---|
| 1 | 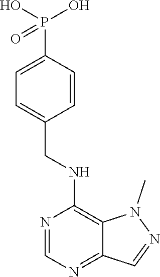 [SMILES: Cn1ncc2ncnc(NCc3ccc(P(=O)(O)O)cc3)c21] | 4-[([1-methylpyrazolo[4,3-d]pyrimidin-7-yl]amino)methyl]-phenyl- phosphonic acid |
| 2 | 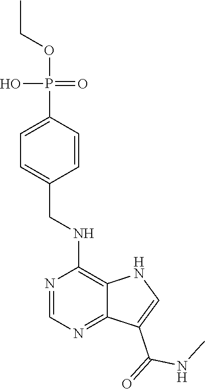 [SMILES: CCOP(=O)(O)c1ccc(CNc2ncnc3c(C(=O)NC)c[nH]c23)cc1] | ethoxy(4-([[7-(methylcarbamoyl)-5H-pyrrolo[3,2-d]pyrimidin-4-yl]- amino]methyl)phenyl)phosphinic acid |
| 3 | 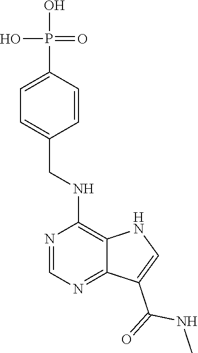 [SMILES: CNC(=O)c1c[nH]c2c(NCc3ccc(P(=O)(O)O)cc3)ncnc12] | 4-([[7-(methylcarbamoyl)-5H-pyrrolo[3,2-d]pyrimidin-4-yl]amino]- methyl)phenylphosphonic acid |
| 4 | 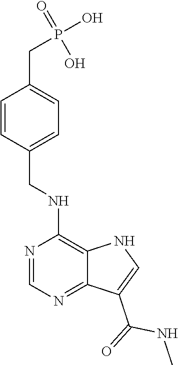 [SMILES: CNC(=O)c1c[nH]c2c(NCc3ccc(CP(=O)(O)O)cc3)ncnc12] | [4-([[7-(methylcarbamoyl)-5H-pyrrolo[3,2-d]pyrimidin-4-yl]amino]- methyl)phenyl]methylphosphonic acid |
| 5 | 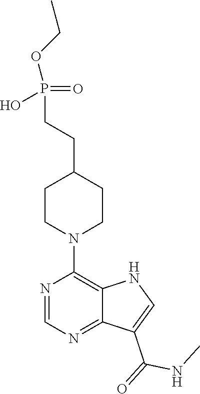 [SMILES: CCOP(=O)(O)CCC1CCN(c2ncnc3c(C(=O)NC)c[nH]c23)CC1] | ethoxy(2-[1-[7-(methylcarbamoyl)-5H-pyrrolo[3,2-d]pyrimidin-4-yl]- piperidin-4-yl]ethyl)phosphinic acid |
| 6 | 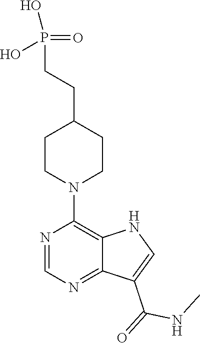 [SMILES: CNC(=O)c1c[nH]c2c(N3CCC(CCP(=O)(O)O)CC3)ncnc12] | 2-[1-[7-(methylcarbamoyl)-5H-pyrrolo[3,2-d]pyrimidin-4-yl]- piperidin-4-yl]ethylphosphonic acid |
| 7 | 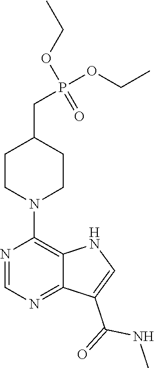 [SMILES: CCOP(=O)(CC1CCN(c2ncnc3c(C(=O)NC)c[nH]c23)CC1)OCC] | diethyl [1-[7-(methylcarbamoyl)-5H-pyrrolo[3,2-d]pyrimidin-4-yl]- piperidin-4-yl]methylphosphonate |
| 8 | 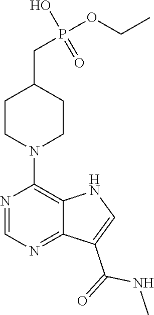 [SMILES: CCOP(=O)(O)CC1CCN(c2ncnc3c(C(=O)NC)c[nH]c23)CC1] | ethoxy([1-[7-(methylcarbamoyl)-5H-pyrrolo[3,2-d]pyrimidin-4-yl]- piperidin-4-yl]methyl)phosphinic acid |
| 9 | 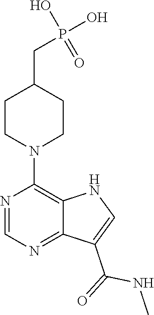 [SMILES: CNC(=O)c1c[nH]c2c(N3CCC(CP(=O)(O)O)CC3)ncnc12] | [1-[7-(methylcarbamoyl)-5H-pyrrolo[3,2-d]pyrimidin-4-yl]piperidin- 4-yl]methylphosphonic acid |
| 10 | 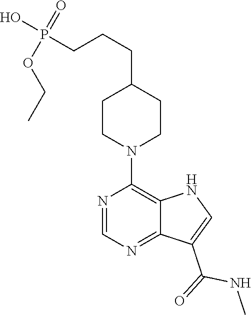 [SMILES: CCOP(=O)(O)CCCC1CCN(c2ncnc3c(C(=O)NC)c[nH]c23)CC1] | ethoxy(3-[1-[7-(methylcarbamoyl)-5H-pyrrolo[3,2-d]pyrimidin-4-yl]- piperidin-4-yl]propyl)phosphinic acid |
| 11 | 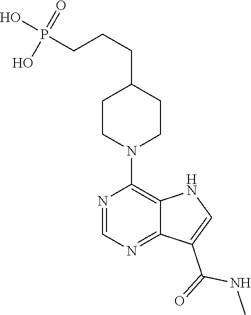 [SMILES: CNC(=O)c1c[nH]c2c(N3CCC(CCCP(=O)(O)O)CC3)ncnc12] | 3-[1-[7-(methylcarbamoyl)-5H-pyrrolo[3,2-d]pyrimidin-4-yl]- piperidin-4-yl]propylphosphonic acid |
| 12 | 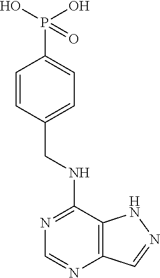 [SMILES: O=P(O)(O)c1ccc(CNc2ncnc3cn[nH]c23)cc1] | 4-([1H-pyrazolo[4,3-d]pyrimidin-7-ylamino]methyl)phenyl- phosphonic acid |
| 13 | 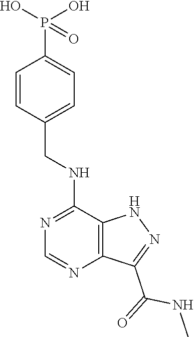 [SMILES: CNC(=O)c1n[nH]c2c(NCc3ccc(P(=O)(O)O)cc3)ncnc12] | 4-([[3-(methylcarbamoyl)-1H-pyrazolo[4,3-d]pyrimidin-7-yl]amino]- methyl)phenylphosphonic acid |
| 14 | 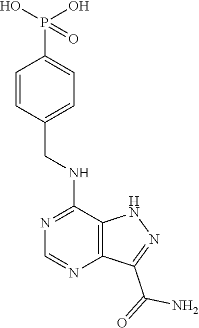 [SMILES: NC(=O)c1n[nH]c2c(NCc3ccc(P(=O)(O)O)cc3)ncnc12] | 4-[([3-carbamoyl-1H-pyrazolo[4,3-d]pyrimidin-7-yl]amino)methyl]- phenylphosphonic acid |
| 15 | 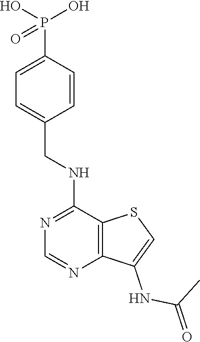 [SMILES: CC(=O)Nc1csc2c(NCc3ccc(P(=O)(O)O)cc3)ncnc12] | 4-[([7-acetamidothieno[3,2-d]pyrimidin-4-yl]amino)methyl]- phenylphosphonic acid |
| 16 | 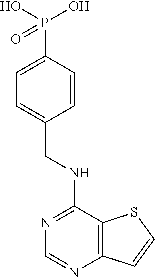 [SMILES: O=P(O)(O)c1ccc(CNc2ncnc3ccsc23)cc1] | 4-([thieno[3,2-d]pyrimidin-4-ylamino]methyl)phenyl-phosphonic acid |
| 17 | 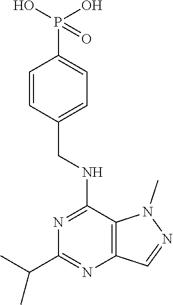 [SMILES: CC(C)c1nc(NCc2ccc(P(=O)(O)O)cc2)c2c(cnn2C)n1] | 4-(((5-isopropyl-1-methyl-1H-pyrazolo[4,3-d]pyrimidin-7- yl)amino)methyl)phenyl-phosphonic acid |
| 18 | 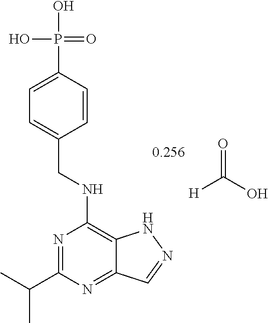 [SMILES: CC(C)c1nc(NCc2ccc(P(=O)(O)O)cc2)c2[nH]ncc2n1 ; O=CO] | 4-[([5-isopropyl-1H-pyrazolo[4,3-d]pyrimidin-7- yl]amino)methyl]phenylphosphonic acid formic acid salt |
| 19 | 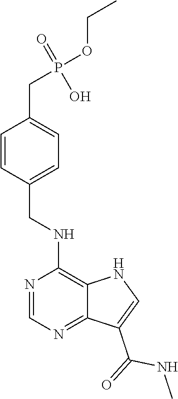 [SMILES: CCOP(=O)(O)Cc1ccc(CNc2ncnc3c(C(=O)NC)c[nH]c23)cc1] | ethoxy([4-([[7-(methylcarbamoyl)-5H-pyrrolo[3,2-d]pyrimidin-4- yl]amino]-methyl)-phenyl]methyl)phosphinic acid |
| 20 | 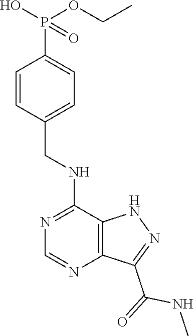 [SMILES: CCOP(=O)(O)c1ccc(CNc2ncnc3c(C(=O)NC)n[nH]c23)cc1] | ethoxy(4-([[3-(methylcarbamoyl)-1H-pyrazolo[4,3-d]pyrimidin-7- yl]amino methyl)-phenyl)phosphinic acid |

### EMBODIMENTS

In embodiments A to R and subembodiments contained therein, the present disclosure includes:

### Embodiment A

In embodiment A, provided is a compound of Formula (I), or a pharmaceutically acceptable salt thereof, where:
alk is alkylene optionally substituted with one to three halo;
one or both of R³ and R⁴ are other than phenyl, heteroaryl, phenylalkenyl, and heteroarylalkenyl (where phenyl, by itself or as part of phenylalkenyl and heteroaryl, by itself or as part of heteroarylalkenyl are optionally substituted with one, two, or three substituents independently selected from alkyl, hydroxy, alkoxy, halo, haloalkyl, haloalkoxy, and cyano); and
R⁵ is absent, alkyl, hydroxy, halo, acylamino, hydroxyalkylamino, alkoxyalkylamino, cyano, aminocarbonyl, alkylaminocarbonyl, or dialkylaminocarbonyl;
and other groups are as defined in the Summary; or
a compound of Formula (IA), or (IB), or a pharmaceutically acceptable salt thereof, as defined in the Summary above.

(Ai) In subembodiment (Ai) of embodiment A, the compounds, or a pharmaceutically acceptable salt thereof, have a structure of Formula (I) in embodiment A above.

(Aii) In subembodiment (Aii) of embodiment A, the compounds, or a pharmaceutically acceptable salt thereof, have a structure of Formula (IA) as defined in the Summary above.

(Aiii) In subembodiment (Aiii) of embodiment A, the compounds, or a pharmaceutically acceptable salt thereof, have a structure of Formula (IB) as defined in the Summary above.

### Embodiment B

In embodiment B, the compounds of any one of embodiment A and subembodiments contained therein (i.e., (Ai), (Aii) and (Aiii)), or a pharmaceutically acceptable salt thereof, have a structure of formula (Ia) or (Ib):

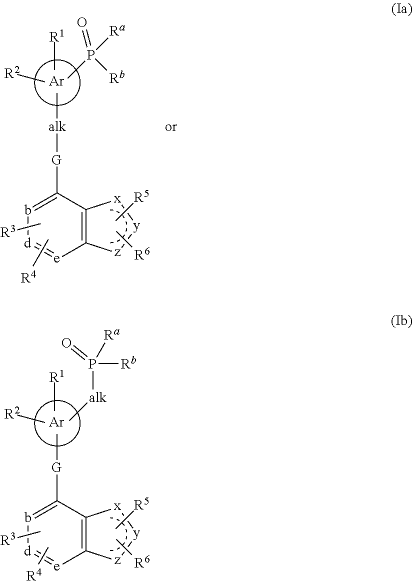

(Bi) In subembodiment (Bi) of embodiment B, the compounds of embodiment B, or a pharmaceutically acceptable salt thereof, have a structure of formula (Ia).

(Bii) In subembodiment (Bii) of embodiment B, the compounds of embodiment B, or a pharmaceutically acceptable salt thereof, have a structure of formula (Ib).

### Embodiment C

In embodiment C, the compounds of any one of embodiment A and subembodiments contained therein (i.e., (Ai), (Aii) and (Aiii)), or a pharmaceutically acceptable salt thereof, have a structure of formula (Ic) or (Id):

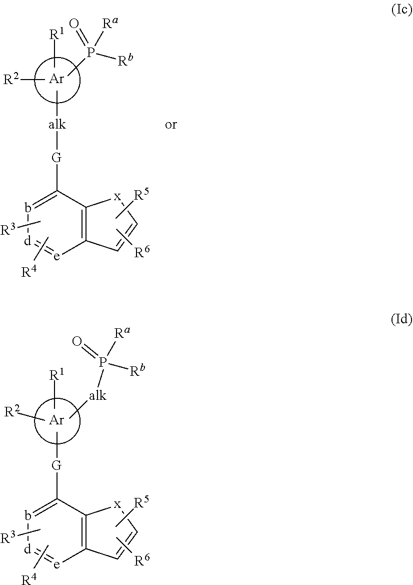

wherein x is NH, O, or S.

(Ci) In subembodiment (Ci) of embodiment C, the compounds of embodiment C, or a pharmaceutically acceptable salt thereof, have a structure of formula (Ic).

(Cii) In subembodiment (Cii) of embodiment C, the compounds of embodiment C, or a pharmaceutically acceptable salt thereof have a structure of formula (Id).

(Ciii) In subembodiment (Ciii) of embodiment C, the compounds of any one of embodiment C and subembodiments (Ci), and (Cii) contained therein, or a pharmaceutically acceptable salt thereof, are wherein x is NH.

(Civ) In subembodiment (Civ) of embodiment C, the compounds of any one of embodiment C and subembodiments (Ci), and (Cii) contained therein, or a pharmaceutically acceptable salt thereof, are wherein x is O.

(Cv) In subembodiment (Cv) of embodiment C, the compounds of any one of embodiment C and subembodiments (Ci), and (Cii) contained therein, or a pharmaceutically acceptable salt thereof, are wherein x is S.

(Cvi) In subembodiment (Cvi) of embodiment C, the compounds of any one of embodiment C and subembodiments (Ci) to (Ciii) contained therein, or a pharmaceutically acceptable salt thereof, are where, when x is NH, then R⁶ is either attached to the nitrogen of NH or the carbon of the 5-membering ring that is adjacent to x, and R⁵ is attached to carbon of the 5-membered ring that is adjacent to a bridgehead carbon, i.e.

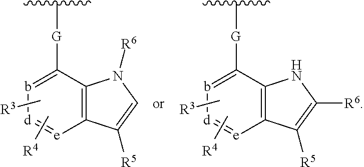

(Cvii) In subembodiment (Cvii) of embodiment C, the compounds of any one of embodiment Cand subembodiments (Ci), (Cii), (Civ) and (Cv) contained therein, or a pharmaceutically acceptable salt thereof, are where, when, x is O or S, R⁶ is attached to the carbon of the 5-membered ring that is adjacent to x and R⁵ when present is attached to carbon of the 5-membered ring that is adjacent to a bridgehead carbon, i.e.

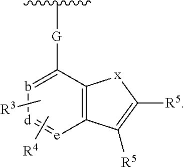

### Embodiment D

In embodiment D, the compound of any one of embodiment A and subembodiments c(Ai), (Aii), and (Aiii) contained therein, or a pharmaceutically acceptable salt thereof, has a structure of formula (Ie) or (If):

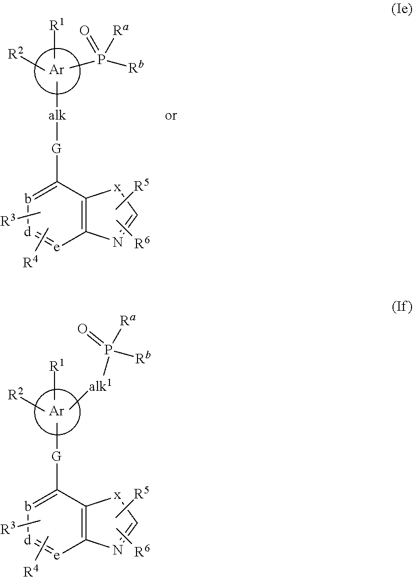

wherein x is NH, O, or S.

(Di) In subembodiment (Di) of embodiment D, the compounds of embodiment D, or a pharmaceutically acceptable salt thereof, have a structure of formula (Ie).

(Dii) In subembodiment (Dii) of embodiment D, the compounds of embodiment D, or a pharmaceutically acceptable salt thereof, have a structure of formula (If).

(Diii) In subembodiment (Diii) of embodiment D, the compounds of any one of embodiment D and subembodiments (Di), and (Dii) contained therein, or a pharmaceutically acceptable salt thereof, are wherein x is NH.

(Div) In subembodiment (Div) of embodiment D, the compounds of any one of embodiment D and subembodiments (Di), and (Dii) contained therein, or a pharmaceutically acceptable salt thereof, are wherein x is O.

(Dv) In subembodiment (Dv) of embodiment D, the compounds of any one of embodiment D and subembodiments (Di), and (Dii) contained therein, or a pharmaceutically acceptable salt thereof, are wherein x is S.

(Dvi) In subembodiment (Dvi) of embodiment D, the compounds of any one of embodiments D and subembodiments (Di) to (Diii) contained therein, or a pharmaceutically acceptable salt thereof, are where, when x is NH, then R⁶ is attached to the nitrogen of NH and R³ is attached to carbon of the 5-membered ring that is adjacent to x, i.e.

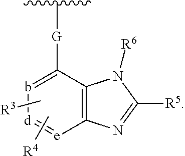

(Dvii) In subembodiment (Dvii) of embodiment D, the compounds of any one of embodiments D and subembodiments (Di), (Dii), (Div) and (Dv) contained therein, or a pharmaceutically acceptable salt thereof, are where, when, x is O or S, R⁶ is absent and R⁵ is attached to carbon of the 5-membered ring that is adjacent to x, i.e.

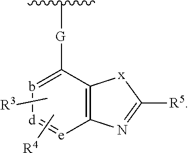

### Embodiment E

In embodiment E, the compounds of any one of embodiment A and subembodiments (Ai), (Aii), and (Aiii) contained therein, or a pharmaceutically acceptable salt thereof, have a structure of formula (Ig) or (Ih):

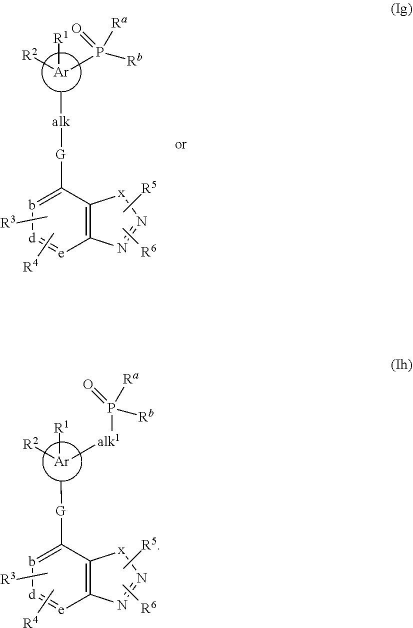

wherein x is NH, O, or S.

(Ei) In subembodiment (Ei) of embodiment E, the compounds of embodiment E, or a pharmaceutically acceptable salt thereof, have a structure of formula (Ig).

(Eii) In subembodiment (Eii) of embodiment E, the compounds of embodiment E, or a pharmaceutically acceptable salt thereof, have as structure of formula (Ih).

(Eiii) In subembodiment (Eiii) of embodiment E, the compounds of any one of embodiment E and subembodiments (Ei), and (Eii) contained therein, or a pharmaceutically acceptable salt thereof, are wherein x is NH.

(Eiv) In subembodiment (Eiv) of embodiment E, the compounds of any one of embodiment E and subembodiments (Ei) and (Eii) contained therein, or a pharmaceutically acceptable salt thereof, are wherein x is O.

(Ev) In subembodiment (Ev) of embodiment E, the compounds of any one of embodiments E and subembodiments (Ei) and (Eii) contained therein, or a pharmaceutically acceptable salt thereof, are wherein x is S.

(Evi) In subembodiment (Evi) of embodiment E, the compounds of any one of embodiment E and subembodiments (Ei) to (Eiii) contained therein, or a pharmaceutically acceptable salt thereof, are where, when x is NH, then R⁶, when present, is attached to the nitrogen of NH and R⁵ is attached to carbon of the 5-membered ring that is adjacent to a bridgehead carbon, i.e.

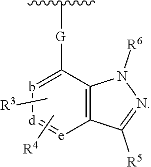

(Evii) In subembodiment (Evii) of embodiment E, the compounds of any one of embodiment E and subembodiments (Ei), (Eii), (Eiv) and (Ev) contained therein, or a pharmaceutically acceptable salt thereof, are where, when, x is O or S, R⁶ is absent and R³ is attached to carbon of the 5-membered ring that is adjacent to a bridgehead carbon, i.e.

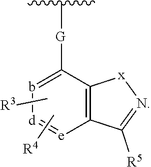

### Embodiment E1

In embodiment E1, the compounds of any one of embodiments A, B, C, D, and E and subembodiments contained therein, or a pharmaceutically acceptable salt thereof, are wherein G is NR, preferably NH.

### Embodiment E2

In embodiment E2, the compounds of any one of embodiments A, B, C, D, and E and subembodiments contained therein, or a pharmaceutically acceptable salt thereof, are wherein G is O.

### Embodiment E3

In embodiment E3, the compounds of any one of embodiments A, B, C, D, and E and subembodiments contained therein, or a pharmaceutically acceptable salt thereof, are wherein G is bond.

### Embodiment F

In embodiment F, the compounds of any one of embodiments A, B, C, D, E, E1, E2, and E3 and subembodiments contained therein, or a pharmaceutically acceptable salt thereof, are wherein b, d, and e are CH or C when attached to any one of R³ and R⁴.

### Embodiment G

In embodiment G, the compounds of any one of embodiments A, B, C, D, E, E1, E2, and E3 and subembodiments contained therein, or a pharmaceutically acceptable salt thereof, are wherein b is N and d, and e are CH or C when attached to any one of R³ and R⁴.

### Embodiment H

In embodiment H, the compounds of any one of embodiments A, B, C, D, E, E1 E2, and E3 and subembodiments contained therein, or a pharmaceutically acceptable salt thereof, are wherein d is N and b and e are CH or C when attached to any one of R³ and R⁴.

### Embodiment I

In embodiment I, the compounds of any one of embodiments A, B, C, D, E, E1, E2, and E3 and subembodiments contained therein, or a pharmaceutically acceptable salt thereof, are wherein e is N and b and d are CH or C when attached to any one of R³ and R⁴.

### Embodiment J

In embodiment J, the compounds of any one of embodiments A, B, C, D, E, E1, E2, and E3 and subembodiments contained therein, or a pharmaceutically acceptable salt thereof, are wherein b and e are N and d is CH or C when attached to any one of R³ and R⁴.

### Embodiment K

In embodiment K, the compounds of any one of embodiments A, B, C, D, E, E1, E2, E3, F, G, H, I, and J and subembodiment contained therein, or a pharmaceutically acceptable salt thereof, are wherein —P(═O)(R^{a})(R^{b}) is —P(═O)(OH)₂.

### Embodiment L

In embodiment L, the compounds of any one of embodiments A, B, C, D, E, E1, E2, E3, F, G, H, I, and J and subembodiment contained therein, or a pharmaceutically acceptable salt thereof, are wherein R^{a} and R^{b} are independently selected from hydroxy, alkoxy, —Oaryl (where aryl is optionally substituted with one to three substituents independently selected from alkyl, halo, haloalkyl, cyano, and nitro), —O—(CH₂)OCOR^{c} (where R^{c} is alkyl), and —O-(alk²)OR^{d} (where alk² is alkylene and R^{d} is alkyl); or
R^{a} and R^{b} together with the phosphorus atom to which they are attached form a ring of formula (a):

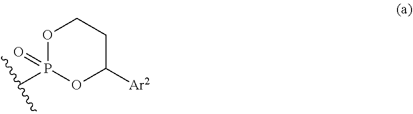

wherein Ar² is phenyl or six membered heteroaryl wherein the phenyl and six membered heteroaryl are optionally substituted with one to three halo. Preferably, R^{a} and R^{b} are independently selected from alkoxy, —Oaryl (where aryl is optionally substituted with one to three substituents independently selected from alkyl, halo, haloalkyl, cyano, and nitro), —O—(CH₂)OCOR^{c} (where R^{c} is alkyl), and —O-(alk²)OR^{d} (where alk² is alkylene and R^{d} is alkyl, such as methyl, isopropyl, n-propyl, isobutyl, or n-butyl). Preferably, R^{a} and R^{b} are independently hydroxy, alkoxy, or —Ophenyl (where phenyl is optionally substituted with one to three substituents independently selected from alkoxy, halo, haloalkyl, cyano, and nitro).

(Li) In subembodiment (Li) of embodiment L, the compounds of embodiment L, or a pharmaceutically acceptable salt thereof, are wherein R^{a} and R^{b} are independently selected from hydroxy and alkoxy.

(Lii) In subembodiment (Lii) of embodiment L, the compounds of embodiment L, or a pharmaceutically acceptable salt thereof, are wherein R^{a} and R^{b} together with the phosphorus atom to which they are attached form a ring of formula (a):

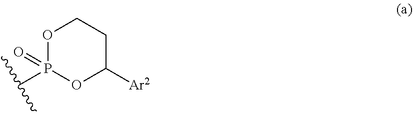

### Embodiment M

In embodiment M, the compounds of any one of embodiments A, B, C, D, E, E1, E2, E3, F, G, H, I, J, K, and L, and subembodiments contained therein or a pharmaceutically acceptable salt thereof, are wherein Ar is aryl, heteroaryl, or heterocyclyl, preferably aryl or heteroaryl.

(Mi). In subembodiment (Mi) of embodiment M, the compounds of embodiment M, or a pharmaceutically acceptable salt thereof, are wherein Ar is phenyl.

(Mii). In subembodiment (Mii) of embodiment M, the compounds of embodiment M, or a pharmaceutically acceptable salt thereof, are wherein Ar is phenyl and —P(═O)(R^{a})(R^{b}) is attached to carbon of the phenyl ring that is meta to the carbon attaching the phenyl ring to remaining compound of Formula (I), (IA), (IB), and (Ia) to (Ih).

(Miii). In subembodiment (Miii) of embodiment M, the compounds of embodiment M, or a pharmaceutically acceptable salt thereof, are wherein Ar is phenyl and —P(═O)(R^{a})(R^{b}) is attached to carbon on the phenyl ring that is para to the carbon attaching the phenyl ring to remaining compound of Formula (I), (IA), (IB), and (Ia) to (Ih), respectively.

(Miv). In subembodiment (Miv) of embodiment M, the compounds of embodiment M, or a pharmaceutically acceptable salt thereof, are wherein Ar is heteroaryl.

(Mv). In subembodiment (Mv) of embodiment M, the compounds of embodiment M, or a pharmaceutically acceptable salt thereof, are wherein Ar is pyridinyl, pyrimidinyl, pyridazinyl, thienyl, furanyl, thiazolyl, oxazolyl, isoxazolyl, pyrazolyl, tiazolyl, oxadiazolyl, or imidazolyl.

(Mvi). In subembodiment (Mvi) of embodiment M, the compounds of embodiment M, or a pharmaceutically acceptable salt thereof, are wherein Ar is a six-membered ring such as pyridinyl, pyrimidinyl, or pyridazinyl wherein —P(═O)(R^{a})(R^{b}) is attached to carbon on the pyridinyl, pyrimidinyl, or pyridazinyl ring that is meta to the carbon attaching the pyridinyl, pyrimidinyl, or pyridazinyl ring to remaining compound of Formula (I), (IA), (IB), and (Ia) to (Ih), respectively.

(Mvii). In subembodiment (Mvii) of embodiment M, the compounds of embodiment M, or a pharmaceutically acceptable salt thereof, are those wherein Ar is benzofuranyl, quinolinyl, quinazolinyl, benzimidazolyl, indazolyl, benzotriazolyl, or benzoxazolyl.

(Mviii). In subembodiment (Mviii) of embodiment M, the compounds of embodiment M, or a pharmaceutically acceptable salt thereof, are those wherein Ar is heterocyclyl e.g., pyrrolidinyl or piperidinyl. In a subembodiment of embodiment (Mviii), Ar is piperidin-1-yl (i.e., piperidinyl ring is attached to the remainder of the molecule of Formula (I), (IA), (IB), and (Ia) to (Ih) via the nitrogen atom of the piperidinyl ring) and —P(═O)(R^{a})(R^{b}) is attached to carbon on the piperidin-1-yl ring that is para to the nitrogen atom of the piperidin-1-yl ring.

### Embodiment N

In embodiment N, the compounds of any one of embodiments A, B, C, D, E, E1, E2, E3, F, G, H, I, J, K, L, and M, and subembodiments contained therein, or a pharmaceutically acceptable salt thereof, are wherein alk and alk¹ are independently methylene, ethylene, or propylene.

(Ni) In subembodiment (Ni) of embodiment N, the compounds of embodiment N, or a pharmaceutically acceptable salt thereof, are those wherein alk and alk¹ are methylene.

### Embodiment O

In embodiment O, the compounds of any one of embodiments A, B, D, E, E1, E2, E3, F, G, H, J, K, L, M, and N and subembodiments contained therein, or a pharmaceutically acceptable thereof, are wherein R⁶ is absent, methyl, or isopropyl.

(Oi). In subembodiment (Oi) of embodiment O, the compounds of embodiment O, or a pharmaceutically acceptable salt thereof, are those wherein R⁶ is absent.

(Oii). In subembodiment (Oii) of embodiment O, the compounds of embodiment O, or a pharmaceutically acceptable salt thereof, are wherein R⁶ is methyl or isopropyl.

### Embodiment P

In embodiment P, the compounds of any one of embodiments A, B, D, E, E1, E2, E3, F, G, H, J, K, L, M, N, and O, and subembodiments contained therein, or a pharmaceutically acceptable thereof, are wherein R⁵ is absent, alkyl, hydroxy, halo, acylamino, alkoxyalkylamino, cyano, aminocarbonyl, alkylaminocarbonyl, or dialkylaminocarbonyl.

(Pi). In subembodiment (Pi) of embodiment P, the compounds of embodiment P, or a pharmaceutically acceptable salt thereof, are wherein R³ is absent or alkyl.

(Pii). In subembodiment (Pii) of embodiment P, the compounds of embodiment P, or a pharmaceutically acceptable salt thereof, are wherein R³ is cyano, acylamino, aminocarbonyl, alkylaminocarbonyl, or dialkylaminocarbonyl. Preferably R³ is acylamino (e.g., methylcarbonylamino, ethylcarbonylamino), aminocarbonyl, methylaminocarbonyl, ethylaminocarbonyl, dimethylaminocarbonyl, or isopropylcarbonyl.

### Embodiment Q

In embodiment Q, the compounds of any one of embodiments A, B, C, D, E, E1, E2, E3, F, G, H, I, J, K, L, M, N, O, and P, and subembodiments contained therein, or a pharmaceutically acceptable thereof, are wherein R¹ and R² are independently absent, methyl, ethyl, methoxy, fluoro, trifluoromethyl, trifluoromethoxy, or cyano.

(Qi). In subembodiment (Qi) of embodiment, Q, the compounds of embodiment Q, or a pharmaceutically acceptable salt thereof, are wherein R¹ and R² are absent.

### Embodiment R

(Ri) In embodiment Ri, the compounds of any one of embodiments A, B, C, D, E, E1, E2, E3, F, G, H, I, J, K, L, M, N, O, P, an Q and subembodiments contained therein, or a pharmaceutically acceptable thereof, are wherein R³ and R⁴ are independently absent, alkyl, alkoxy, hydroxy, amino, halo, haloalkyl, or haloalkoxy.

(Ri1). In subembodiment (Ri1) of embodiment (Ri), the compounds of embodiment (Ri), or a pharmaceutically acceptable thereof, are wherein R³ and R⁴ are independently absent, alkyl, alkoxy, amino, or hydroxy.

(Ri2). In subembodiment (Ri2) of embodiment (Ri), the compounds of embodiment (Ri), or pharmaceutically acceptable thereof, are wherein R³ and R⁴ are independently absent, alkyl, alkoxy, amino, or hydroxy, preferably, R³ and R⁴ are independently absent, methyl, ethyl, isopropyl, hydroxy, methoxy, ethoxy, or propoxy and R⁴, is present and is attached to the six membered ring comprising b, d, and e of Formula (I), (IA), (IB), and (Ia) to (Ih) as shown below:

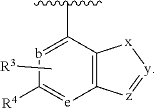

wherein the wavy line denotes the attachment point to the remainder of the molecule.

(Ri3). In subembodiment (Ri3) of embodiment (Ri), the compounds of embodiment (Ri), or pharmaceutically acceptable thereof, are wherein R³ and R⁴ are absent.

(Ri4). In subembodiment (Ri4) of embodiment (Ri), the compounds of embodiment (Ri), or pharmaceutically acceptable thereof, are those wherein when R⁴ is present, R⁴ is attached to the six membered ring comprising b, d, and e of Formula (I), (IA), (IB), and (Ia) to (Ih) as shown below:

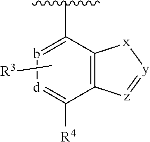

wherein the wavy line denotes the attachment point to the remainder of the molecule.

(Rii). In embodiment (Rii), the compounds of any one of embodiments A, B, C, D, E, E1, E2, E3, F, G, H, I, J, K, L, M, N, O, P, an Q and subembodiments contained therein, or a pharmaceutically acceptable thereof, are wherein:
R³ is absent, alkyl, alkoxy, hydroxy, halo, haloalkyl, or haloalkoxy; and
R⁴ is hydroxyalkyl, alkoxyalkyl, hydroxyalkoxy, alkoxyalkoxy, hydroxyalkylamino, alkoxyalkylamino, aminoalkyl, aminoalkoxy, aminoalkylamino, heterocyclyl, heterocyclyloxy, heterocyclylamino (wherein heterocyclyl either alone or part of heterocyclyloxy and heterocyclylamino is optionally substituted with one, two, or three substituents independently selected from alkyl, halo, hydroxy, alkoxy, hydroxyalkyl, alkoxyalkyl, and aminoalkyl), heterocyclylalkyl, heterocyclylalkyloxy, heterocyclylalkylamino (wherein the heterocyclyl ring in heterocyclylalkyl, heterocyclylalkyloxy, and heterocyclylalkylamino is optionally substituted with one, two, or three substituents independently selected from alkyl, halo, hydroxy, alkoxy, hydroxyalkyl, alkoxyalkyl, and aminoalkyl), cycloalkyloxy, phenyloxy, and heteroaryloxy (where phenyl in phenyloxy and heteroaryl in heteroaryloxy are optionally substituted with one, two, or three substituents, preferably one or two optional substituents, where two of the optional substituents are independently selected from alkyl, hydroxy, alkoxy, halo, haloalkyl, haloalkoxy, and cyano).

In a first subembodiment of embodiment (Rii), the compounds of embodiment (Rii), or pharmaceutically acceptable thereof, are wherein R³ is absent, methoxy, ethoxy, or hydroxy, preferably R³ is methoxy or ethoxy; and R⁴ is 2-hydroxyethyloxy, 3-hydroxypropyloxy, 2-methoxyethyloxy, 2-ethoxyethyloxy, 3-methoxypropyloxy, 3-ethoxypropyloxy, 2-aminoethyloxy, 2-methylaminoethyloxy, 2-dimethylaminoethyloxy, 2-diethylaminoethyloxy, 3-aminopropyloxy, 3-methylaminopropyloxy, 3-dimethylaminopropyloxy, 3-diethylaminopropyloxy, pyrrolidinyloxy, piperidinyloxy, pyrrolidinylmethyloxy, piperidinylmethyloxy, pyrrolidinylethyloxy, piperidinylethyloxy, 2-hydroxyethylamino, 3-hydroxypropylamino, 2-methoxyethylamino, 2-ethoxyethylamino, 3-methoxypropylamino, 3-ethoxypropylamino, 2-aminoethylamino, 2-methylaminoethylamino, 2-dimethylaminoethylamino, 2-diethylaminoethylamino, 3-aminopropylamino, 3-methylaminopropylamino, 3-dimethylaminopropylamino, 3-diethylaminopropylamino, pyrrolidinylamino, piperidinylamino, pyrrolidinylmethylamino, piperidinylmethylamino, pyrrolidinylethylamino, or piperidinylethylamino (wherein pyrrolidinyl and piperidinyl in each of aforementioned groups, alone or part of another group is optionally substituted with one or two substituents independently selected from methyl, fluoro, hydroxy, and methoxy). Preferably, R³ and R⁴ are attached to the six membered ring comprising b, d, and e of Formula (I), (A), (IB), and (Ia) to (Ih) as shown below

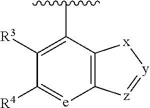

wherein the wavy line denotes the attachment point to the remainder of the molecule.

(Riii). In embodiment (Riii), the compounds of any one of embodiments A, B, C, D, E, E1, E2, E3, F, G, H, I, J, K, L, M, N, O, P, an Q and subembodiments contained therein, or a pharmaceutically acceptable thereof, are wherein R³ and R⁴ are independently hydroxyalkyl, alkoxyalkyl, hydroxyalkoxy, alkoxyalkoxy, hydroxyalkylamino, alkoxyalkylamino, aminoalkyl, aminoalkoxy, aminoalkylamino, heterocyclyl, heterocyclyloxy, heterocyclylamino (wherein heterocyclyl either alone or part of heterocyclyloxy and heterocyclylamino is optionally substituted with one, two, or three substituents independently selected from alkyl, halo, hydroxy, alkoxy, hydroxyalkyl, alkoxyalkyl, and aminoalkyl), heterocyclylalkyl, heterocyclylalkyloxy, heterocyclylalkylamino (wherein the heterocyclyl ring in heterocyclylalkyl, heterocyclylalkyloxy, and heterocyclylalkylamino is optionally substituted with one, two, or three substituents independently selected from alkyl, halo, hydroxy, alkoxy, hydroxyalkyl, alkoxyalkyl, and aminoalkyl), cycloalkyloxy, phenyloxy, and heteroaryloxy (where phenyl of phenyloxy and heteroaryl of heteroaryloxy are optionally substituted with one, two, or three substituents, preferably one or two optional substituents, where two of the optional substituents are independently selected from alkyl, hydroxy, alkoxy, halo, haloalkyl, haloalkoxy, and cyano).

In a first subembodiment of embodiment (Riii), the compounds of embodiment (Riii), or pharmaceutically acceptable thereof, are wherein R³ and R⁴ are independently 2-hydroxyethyloxy, 3-hydroxypropyloxy, 2-methoxyethyloxy, 2-ethoxyethyloxy, 3-methoxypropyloxy, 3-ethoxypropyloxy, 2-aminoethyloxy, 2-methylaminoethyloxy, 2-dimethylaminoethyloxy, 2-diethylaminoethyloxy, 3-aminopropyloxy, 3-methylaminopropyloxy, 3-dimethylaminopropyloxy, 3-diethylaminopropyloxy, pyrrolidinyloxy, piperidinyloxy, pyrrolidinylmethyloxy, piperidinylmethyloxy, pyrrolidinylethyloxy, piperidinylethyloxy, 2-hydroxyethylamino, 3-hydroxypropylamino, 2-methoxyethylamino, 2-ethoxyethylamino, 3-methoxypropylamino, 3-ethoxypropylamino, 2-aminoethylamino, 2-methylaminoethylamino, 2-dimethylaminoethylamino, 2-diethylaminoethylamino, 3-aminopropylamino, 3-methylaminopropylamino, 3-dimethylaminopropylamino, 3-diethylaminopropylamino, pyrrolidinylamino, piperidinylamino, pyrrolidinylmethylamino, piperidinylmethylamino, pyrrolidinylethylamino, or piperidinylethylamino (wherein pyrrolidinyl and piperidinyl in each of aforementioned groups, alone or part of another group is optionally substituted with one or two substituents independently selected from methyl, fluoro, hydroxy, and methoxy). Preferably, R³ and R⁴ are attached to the six membered ring comprising b, d, and e of Formula (I), (IA), (IB), and (Ia) to (Ih) as shown below:

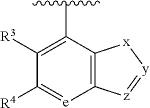

wherein the wavy line denotes the attachment point to the remainder of the molecule.

Further embodiments A1 to R1, the present disclosure includes:

### Embodiment A1

In embodiment A1, provided is a compound of Formula (I), or a pharmaceutically acceptable salt thereof, where alk is alkynylene; and other groups are as defined in the Summary.

### Embodiment B1

In embodiment B1, the compounds of any one of embodiment A1, or a pharmaceutically acceptable salt thereof, have a structure of formula (Ia1):

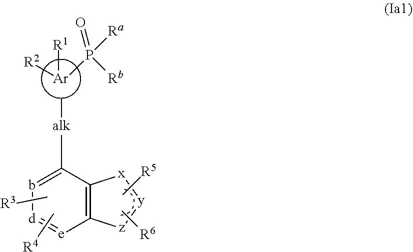

where X is N, O, or S.

### Embodiment C1

In embodiment C1, the compounds of embodiment A, or a pharmaceutically acceptable salt thereof, have a structure of formula (Ic1):

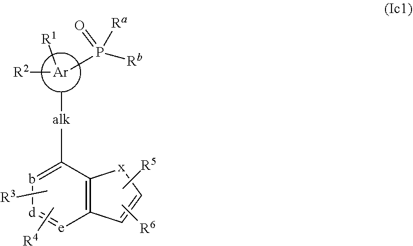

wherein x is NH, O, or S.

(C1i) In subembodiment (C1i) of embodiment C1, or a pharmaceutically acceptable salt thereof, are wherein x is NH.

(C1ii) In subembodiment (C1ii) of embodiment C1, or a pharmaceutically acceptable salt thereof, are wherein x is O.

(C1iii) In subembodiment (C1iii) of embodiment C1, or a pharmaceutically acceptable salt thereof, are wherein x is S.

(C1iv) In subembodiment (C1iv) of embodiment C1, the compounds of any one of embodiment C1 and subembodiments (C1i) to (C1iii) contained therein, or a pharmaceutically acceptable salt thereof, are where, when x is NH, then R⁶ is either attached to the nitrogen of NH or the carbon of the 5-membering ring that is adjacent to x, and R⁵ is attached to carbon of the 5-membered ring that is adjacent to a bridgehead carbon, i.e.

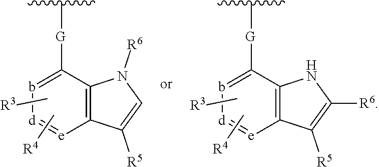

(C1vi) In subembodiment (C1v) of embodiment C1, the compounds of any one of embodiment C1 and subembodiments (C1ii), and (C1iii) contained therein, or a pharmaceutically acceptable salt thereof, are where, when, x is O or S, R⁶ is attached to the carbon of the 5-membered ring that is adjacent to x and R⁵ when present is attached to carbon of the 5-membered ring that is adjacent to a bridgehead carbon, i.e.

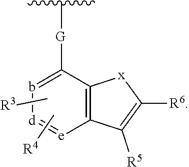

### Embodiment D1

In embodiment D, the compound of embodiment A1, or a pharmaceutically acceptable salt thereof, has a structure of formula (Ie1):

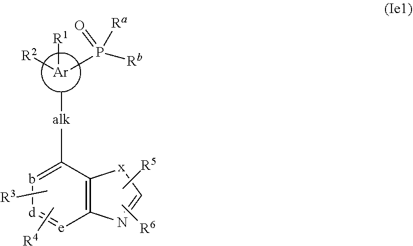

wherein x is NH, O, or S.

(D1i) In subembodiment (D1i) of embodiment D1, the compounds of embodiment D1, or a pharmaceutically acceptable salt thereof, are wherein x is NH.

(D1ii) In subembodiment (D1ii) of embodiment D1, the compounds of embodiment D1, or a pharmaceutically acceptable salt thereof, are wherein x is O.

(D1iii) In subembodiment (D1iii) of embodiment D1, or a pharmaceutically acceptable salt thereof, are wherein x is S.

(D1iv) In subembodiment (D1iv) of embodiment D1, the compounds of any one of embodiments D1 and subembodiment (D1i), or a pharmaceutically acceptable salt thereof, are where, when x is NH, then R⁶ is attached to the nitrogen of NH and R⁵ is attached to carbon of the 5-membered ring that is adjacent to x, i.e.

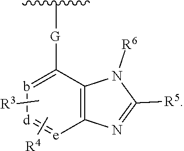

(D1v) In subembodiment (D1v) of embodiment D1, the compounds of any one of embodiments D and subembodiments (D1ii) and (D1iii) contained therein, or a pharmaceutically acceptable salt thereof, are where, when, x is O or S, R⁶ is absent and R⁵ is attached to carbon of the 5-membered ring that is adjacent to x, i.e.

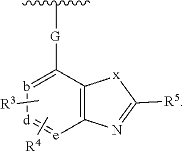

### Embodiment E1

In embodiment E1, the compounds of embodiment A1, or a pharmaceutically acceptable salt thereof, have a structure of formula (Ig1):

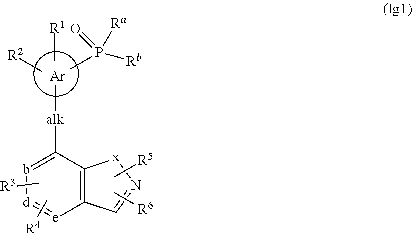

wherein x is NH, O, or S.

(E1i) In subembodiment (E1i) of embodiment E1, the compounds of any one of embodiment E1, or a pharmaceutically acceptable salt thereof, are wherein x is NH.

(E1ii) In subembodiment (E1ii) of embodiment E1, the compounds of any one of embodiment E1, or a pharmaceutically acceptable salt thereof, are wherein x is O.

(E1iii) In subembodiment (E1iii) of embodiment E1, or a pharmaceutically acceptable salt thereof, are wherein x is S.

(E1iv) In subembodiment (E1iv) of embodiment E1, the compounds of any one of embodiment E1 and subembodiment (E1i) contained therein, or a pharmaceutically acceptable salt thereof, are where, when x is NH, then R⁶, when present, is attached to the nitrogen of NH and R⁵ is attached to carbon of the 5-membered ring that is adjacent to a bridgehead carbon, i.e.

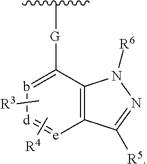

(E1v) In subembodiment (E1v) of embodiment E1, the compounds of any one of embodiment E1 and subembodiments (E1ii) and (E1iii) contained therein, or a pharmaceutically acceptable salt thereof, are where, when, x is O or S, R⁶ is absent and R⁵ is attached to carbon of the 5-membered ring that is adjacent to a bridgehead carbon, i.e.

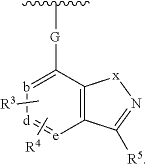

### Embodiment F1

In embodiment F1, the compounds of any one of embodiments A1, B1, C1, D1, and E1, and subembodiments contained therein, or a pharmaceutically acceptable salt thereof, are wherein b, d, and e are CH or C when attached to any one of R³ and R⁴.

### Embodiment G1

In embodiment G1, the compounds of any one of embodiments A1, B1, C1, D1, and E1, and subembodiments contained therein, or a pharmaceutically acceptable salt thereof, are wherein b is N and d, and e are CH or C when attached to any one of R³ and R⁴.

### Embodiment H1

In embodiment H1, the compounds of any one of embodiments A1, B1, C1, D1, and E1, and subembodiments contained therein, or a pharmaceutically acceptable salt thereof, are wherein d is N and b and e are CH or C when attached to any one of R³ and R⁴.

### Embodiment I1

In embodiment I1, the compounds of any one of embodiments A1, B1, C1, D1, and E1, and subembodiments contained therein, or a pharmaceutically acceptable salt thereof, are wherein e is N and b and d are CH or C when attached to any one of R³ and R⁴.

### Embodiment J1

In embodiment J1, the compounds of any one of embodiments A1, B1, C1, D1, and E1 and subembodiments contained therein, or a pharmaceutically acceptable salt thereof, are wherein b and e are N and d is CH or C when attached to any one of R³ and R⁴.

### Embodiment K1

In embodiment K1, the compounds of any one of embodiments A1, B1, C1, D1, E1, F1, G1, H1, I1, and J1, and subembodiment contained therein, or a pharmaceutically acceptable salt thereof, are wherein —P(═O)(R^{a})(R^{b}) is —P(═O)(OH)₂.

### Embodiment L1

In embodiment L1, the compounds of any one of embodiments A1, B1, C1, D1, E1, F1, G1, H1, I1, and J1, and subembodiment contained therein, or a pharmaceutically acceptable salt thereof, are wherein R^{a} and R^{b} are independently selected from hydroxy, alkoxy, —Oaryl (where aryl is optionally substituted with one to three substituents independently selected from alkyl, halo, haloalkyl, cyano, and nitro), —O—(CH₂)OCOR^{c} (where R^{c} is alkyl), and —O-(alk²)OR^{d} (where alk² is alkylene and R^{d} is alkyl); or
R^{a} and R^{b} together with the phosphorus atom to which they are attached form a ring of formula (a):

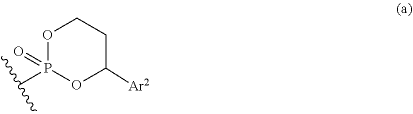

wherein Ar² is phenyl or six membered heteroaryl wherein the phenyl and six membered heteroaryl are optionally substituted with one to three halo. Preferably, R^{a} and R^{b} are independently selected from alkoxy, —Oaryl (where aryl is optionally substituted with one to three substituents independently selected from alkyl, halo, haloalkyl, cyano, and nitro), —O—(CH₂)OCOR^{c} (where R^{c} is alkyl), and —O-(alk²)OR^{d} (where alk² is alkylene and R^{d} is alkyl, such as methyl, isopropyl, n-propyl, isobutyl, or n-butyl). Preferably, R^{a} and R^{b} are independently hydroxy, alkoxy, or —Ophenyl (where phenyl is optionally substituted with one to three substituents independently selected from alkoxy, halo, haloalkyl, cyano, and nitro).

(L1i) In subembodiment (L1i) of embodiment L, the compounds of embodiment L1, or a pharmaceutically acceptable salt thereof, are wherein R^{a} and R^{b} are independently selected from hydroxy and alkoxy.

(L1ii) In subembodiment (L1ii) of embodiment L, the compounds of embodiment L1, or a pharmaceutically acceptable salt thereof, are wherein R^{a} and R^{b} together with the phosphorus atom to which they are attached form a ring of formula (a):

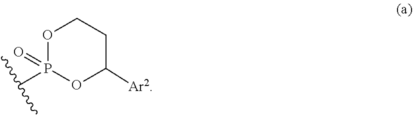

### Embodiment M1

In embodiment M, the compounds of any one of embodiments A1, B1, C1, D1, E1 F1, G1, H1, I1, J1 K1, and L1, and subembodiments contained therein or a pharmaceutically acceptable salt thereof, are wherein Ar is aryl, heteroaryl, or heterocyclyl, preferably aryl or heteroaryl.

(M1i). In subembodiment (M1i) of embodiment M1, the compounds of embodiment M1, or a pharmaceutically acceptable salt thereof, are wherein Ar is phenyl.

(M1ii). In subembodiment (M1ii) of embodiment M1, the compounds of embodiment M1 or a pharmaceutically acceptable salt thereof, are wherein Ar is phenyl and —P(═O)(R^{a})(R^{b}) is attached to carbon of the phenyl ring that is meta to the carbon attaching the phenyl ring to remaining compound of Formula (I), and (Ia1) to (Ig1).

(M1iii). In subembodiment (M1iii) of embodiment M1, the compounds of embodiment M1, or a pharmaceutically acceptable salt thereof, are wherein Ar is phenyl and —P(═O)(R^{a})(R^{b}) is attached to carbon on the phenyl ring that is para to the carbon attaching the phenyl ring to remaining compound of Formula (I), and (Ia1) to (Ig1), respectively.

(M11iv). In subembodiment (M1iv) of embodiment M1, the compounds of embodiment M1, or a pharmaceutically acceptable salt thereof, are wherein Ar is heteroaryl.

(M1v). In subembodiment (M1v) of embodiment M1, the compounds of embodiment M1, or a pharmaceutically acceptable salt thereof, are wherein Ar is pyridinyl, pyrimidinyl, pyridazinyl, thienyl, furanyl, thiazolyl, oxazolyl, isoxazolyl, pyrazolyl, triazolyl, oxadiazolyl, or imidazolyl.

(M1vi). In subembodiment (M1vi) of embodiment M1, the compounds of embodiment M1, or a pharmaceutically acceptable salt thereof, are wherein Ar is a six-membered ring such as pyridinyl, pyrimidinyl, or pyridazinyl wherein —P(═O)(R^{a})(R^{b}) is attached to carbon on the pyridinyl, pyrimidinyl, or pyridazinyl ring that is meta to the carbon attaching the pyridinyl, pyrimidinyl, or pyridazinyl ring to remaining compound of Formula (I), and (Ia1) to (Ig1), respectively.

(M1vii). In subembodiment (M1vii) of embodiment M1, the compounds of embodiment M1, or a pharmaceutically acceptable salt thereof, are those wherein Ar is benzofuranyl, quinolinyl, quinazolinyl, benzimidazolyl, indazolyl, benzotriazolyl, or benzoxazolyl.

(M1viii). In subembodiment (M1viii) of embodiment M1, the compounds of embodiment M1, or a pharmaceutically acceptable salt thereof, are those wherein Ar is heterocyclyl e.g., pyrrolidinyl or piperidinyl. In a subembodiment of embodiment (M1viii), Ar is piperidin-1-yl (i.e., piperidinyl ring is attached to the remainder of the molecule of Formula (I), and (Ia1) to (Ig1) via the nitrogen atom of the piperidinyl ring) and —P(═O)(R⁸)(R^{b}) is attached to carbon on the piperidin-1-yl ring that is para to the nitrogen atom of the piperidin-1-yl ring.

### Embodiment N1

In embodiment N1, the compounds of any one of embodiments A1, B1, C1, D1, E1 F1, G1, H1, I1, J1, K1, L1, and M1, and subembodiments contained therein, or a pharmaceutically acceptable salt thereof, are wherein alk is ethenylene.

### Embodiment O1

In embodiment O1, the compounds of any one of embodiments A1, B1, C1, D1, E1 F1, G1, H1, I1, J1, K1, L1, M1, and N1, and subembodiments contained therein, or a pharmaceutically acceptable thereof, are wherein R⁶ is absent, methyl, or isopropyl.

(O1i). In subembodiment (O1i) of embodiment O1, the compounds of embodiment O1, or a pharmaceutically acceptable salt thereof, are those wherein R⁶ is absent.

(O1ii). In subembodiment (O1ii) of embodiment O1, the compounds of embodiment O1, or a pharmaceutically acceptable salt thereof, are wherein R⁶ is methyl or isopropyl.

### Embodiment P1

In embodiment P1, the compounds of any one of embodiments A1, B1, C1, D1, E1 F1, G1, H1, I1, J1, K1, L1, M1, N1, and 01, and subembodiments contained therein, or a pharmaceutically acceptable thereof, are wherein R⁵ is absent, alkyl, hydroxy, halo, acylamino, alkoxyalkylamino, cyano, aminocarbonyl, alkylaminocarbonyl, or dialkylaminocarbonyl.

(P1i). In subembodiment (P1i) of embodiment P1, the compounds of embodiment P1, or a pharmaceutically acceptable salt thereof, are wherein R³ is absent or alkyl.

(P1ii). In subembodiment (P1ii) of embodiment P1, the compounds of embodiment P1, or a pharmaceutically acceptable salt thereof, are wherein R³ is cyano, acylamino, aminocarbonyl, alkylaminocarbonyl, or dialkylaminocarbonyl. Preferably R³ is acylamino (e.g., methylcarbonylamino, ethylcarbonylamino), aminocarbonyl, methylaminocarbonyl, ethylaminocarbonyl, dimethylaminocarbonyl, or isopropylcarbonyl.

### Embodiment Q1

In embodiment Q, the compounds of any one of embodiments A1, B1, C1, D1, E1 F1, G1, H1, I1, J1, K1, L1, M1, N1, O1, and P1, and subembodiments contained therein, or a pharmaceutically acceptable thereof, are wherein R¹ and R² are independently absent, methyl, ethyl, methoxy, fluoro, trifluoromethyl, trifluoromethoxy, or cyano.

(Q1i). In subembodiment (Q1i) of embodiment, Q1, the compounds of embodiment Q1, or a pharmaceutically acceptable salt thereof, are wherein R¹ and R² are absent.

### Embodiment R1

(R1i) In embodiment R1i, the compounds of any one of embodiments A1, B1, C1, D1, E1 F1, G1, H1, I1, J1, K1, L1, M1, N1, O1, P1, an Q1, and subembodiments contained therein, or a pharmaceutically acceptable thereof, are wherein R³ and R⁴ are independently absent, alkyl, alkoxy, hydroxy, amino, halo, haloalkyl, or haloalkoxy.

(R1i1). In subembodiment (R1i1) of embodiment (R1i), the compounds of embodiment (R1i), or a pharmaceutically acceptable thereof, are wherein R³ and R⁴ are independently absent, alkyl, alkoxy, amino, or hydroxy.

(R1i2). In subembodiment (R1i2) of embodiment (R1i), the compounds of embodiment (R1i), or pharmaceutically acceptable thereof, are wherein R³ and R⁴ are independently absent, alkyl, alkoxy, amino, or hydroxy, preferably, R³ and R⁴ are independently absent, methyl, ethyl, isopropyl, hydroxy, methoxy, ethoxy, or propoxy and R⁴, is present and is attached to the six membered ring comprising b, d, and e of Formula (I), and (Ia1) to (Ig1) as shown below:

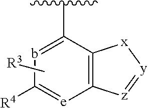

wherein the wavy line denotes the attachment point to the remainder of the molecule.

(R1i3). In subembodiment (R1i3) of embodiment (R1i), the compounds of embodiment (R1i), or pharmaceutically acceptable thereof, are wherein R³ and R⁴ are absent.

(R1i4). In subembodiment (R1i4) of embodiment (R1i), the compounds of embodiment (R1i), or pharmaceutically acceptable thereof, are those wherein when R⁴ is present, R⁴ is attached to the six membered ring comprising b, d, and e of Formula (I), and (Ia1) to (Ig1) as shown below:

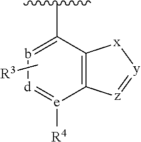

wherein the wavy line denotes the attachment point to the remainder of the molecule.

(R1ii). In embodiment (Rii), the compounds of any one of embodiments A1, B1, C1, D1, E1 F1, G1, H1, I1, J1, K1, L1, M1, N1, O1, P1, an Q1, and subembodiments contained therein, or a pharmaceutically acceptable thereof, are wherein:
R³ is absent, alkyl, alkoxy, hydroxy, halo, haloalkyl, or haloalkoxy; and
R⁴ is hydroxyalkyl, alkoxyalkyl, hydroxyalkoxy, alkoxyalkoxy, hydroxyalkylamino, alkoxyalkylamino, aminoalkyl, aminoalkoxy, aminoalkylamino, heterocyclyl, heterocyclyloxy, heterocyclylamino (wherein heterocyclyl either alone or part of heterocyclyloxy and heterocyclylamino is optionally substituted with one, two, or three substituents independently selected from alkyl, halo, hydroxy, alkoxy, hydroxyalkyl, alkoxyalkyl, and aminoalkyl), heterocyclylalkyl, heterocyclylalkyloxy, heterocyclylalkylamino (wherein the heterocyclyl ring in heterocyclylalkyl, heterocyclylalkyloxy, and heterocyclylalkylamino is optionally substituted with one, two, or three substituents independently selected from alkyl, halo, hydroxy, alkoxy, hydroxyalkyl, alkoxyalkyl, and aminoalkyl), cycloalkyloxy, phenyloxy, and heteroaryloxy (where phenyl in phenyloxy and heteroaryl in heteroaryloxy are optionally substituted with one or two substituents independently selected from alkyl, hydroxy, alkoxy, halo, haloalkyl, haloalkoxy, and cyano).

In a first subembodiment of embodiment (R1ii), the compounds of embodiment (Rii), or pharmaceutically acceptable thereof, are wherein R³ is absent, methoxy, ethoxy, or hydroxy, preferably R³ is methoxy or ethoxy; and R⁴ is 2-hydroxyethyloxy, 3-hydroxypropyloxy, 2-methoxyethyloxy, 2-ethoxyethyloxy, 3-methoxypropyloxy, 3-ethoxypropyloxy, 2-aminoethyloxy, 2-methylaminoethyloxy, 2-dimethylaminoethyloxy, 2-diethylaminoethyloxy, 3-aminopropyloxy, 3-methylaminopropyloxy, 3-dimethylaminopropyloxy, 3-diethylaminopropyloxy, pyrrolidinyloxy, piperidinyloxy, pyrrolidinylmethyloxy, piperidinylmethyloxy, pyrrolidinylethyloxy, piperidinylethyloxy, 2-hydroxyethylamino, 3-hydroxypropylamino, 2-methoxyethylamino, 2-ethoxyethylamino, 3-methoxypropylamino, 3-ethoxypropylamino, 2-aminoethylamino, 2-methylaminoethylamino, 2-dimethylaminoethylamino, 2-diethylaminoethylamino, 3-aminopropylamino, 3-methylaminopropylamino, 3-dimethylaminopropylamino, 3-diethylaminopropylamino, pyrrolidinylamino, piperidinylamino, pyrrolidinylmethylamino, piperidinylmethylamino, pyrrolidinylethylamino, or piperidinylethylamino (wherein pyrrolidinyl and piperidinyl in each of aforementioned groups, alone or part of another group is optionally substituted with one or two substituents independently selected from methyl, fluoro, hydroxy, and methoxy). Preferably, R³ and R⁴ are attached to the six membered ring comprising b, d, and e of Formula (I), and (Ia1) to (Ig1) as shown below

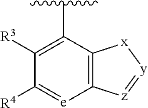

wherein the wavy line denotes the attachment point to the remainder of the molecule.

(R1iii). In embodiment (R1iii), the compounds of any one of embodiments A1, B1, C1, D1, E1 F1, G1, H1, I1, J1, K1, L1, M1, N1, O1, P1, and Q1, and subembodiments contained therein, or a pharmaceutically acceptable thereof, are wherein R³ and R⁴ are independently hydroxyalkyl, alkoxyalkyl, hydroxyalkoxy, alkoxyalkoxy, hydroxyalkylamino, alkoxyalkylamino, aminoalkyl, aminoalkoxy, aminoalkylamino, heterocyclyl, heterocyclyloxy, heterocyclylamino (wherein heterocyclyl either alone or part of heterocyclyloxy and heterocyclylamino is optionally substituted with one, two, or three substituents independently selected from alkyl, halo, hydroxy, alkoxy, hydroxyalkyl, alkoxyalkyl, and aminoalkyl), heterocyclylalkyl, heterocyclylalkyloxy, heterocyclylalkylamino (wherein the heterocyclyl ring in heterocyclylalkyl, heterocyclylalkyloxy, and heterocyclylalkylamino is optionally substituted with one, two, or three substituents independently selected from alkyl, halo, hydroxy, alkoxy, hydroxyalkyl, alkoxyalkyl, and aminoalkyl), cycloalkyloxy, phenyloxy, and heteroaryloxy (where phenyl of phenyloxy and heteroaryl of heteroaryloxy are optionally substituted with one or two 1 substituents, independently selected from alkyl, hydroxy, alkoxy, halo, haloalkyl, haloalkoxy, and cyano).

In a first subembodiment of embodiment (R1iii), the compounds of embodiment (R1iii), or pharmaceutically acceptable thereof, are wherein R³ and R⁴ are independently 2-hydroxyethyloxy, 3-hydroxypropyloxy, 2-methoxyethyloxy, 2-ethoxyethyloxy, 3-methoxypropyloxy, 3-ethoxypropyloxy, 2-aminoethyloxy, 2-methylaminoethyloxy, 2-dimethylaminoethyloxy, 2-diethylaminoethyloxy, 3-aminopropyloxy, 3-methylaminopropyloxy, 3-dimethylaminopropyloxy, 3-diethylaminopropyloxy, pyrrolidinyloxy, piperidinyloxy, pyrrolidinylmethyloxy, piperidinylmethyloxy, pyrrolidinylethyloxy, piperidinylethyloxy, 2-hydroxyethylamino, 3-hydroxypropylamino, 2-methoxyethylamino, 2-ethoxyethylamino, 3-methoxypropylamino, 3-ethoxypropylamino, 2-aminoethylamino, 2-methylaminoethylamino, 2-dimethylaminoethylamino, 2-diethylaminoethylamino, 3-aminopropylamino, 3-methylaminopropylamino, 3-dimethylaminopropylamino, 3-diethylaminopropylamino, pyrrolidinylamino, piperidinylamino, pyrrolidinylmethylamino, piperidinylmethylamino, pyrrolidinylethylamino, or piperidinylethylamino (wherein pyrrolidinyl and piperidinyl in each of aforementioned groups, alone or part of another group is optionally substituted with one or two substituents independently selected from methyl, fluoro, hydroxy, and methoxy). Preferably, R³ and R⁴ are attached to the six membered ring comprising b, d, and e of Formula (I), and (Ia1) to (Ig1) as shown below:

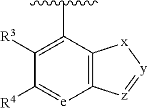

wherein the wavy line denotes the attachment point to the remainder of the molecule.

### General Synthetic Scheme

Compounds of this disclosure can be made by the methods depicted in the reaction schemes shown below.

The starting materials and reagents used in preparing these compounds are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this disclosure can be synthesized, and various modifications to these schemes can be made and will be suggested to one skilled in the art reading this disclosure. The starting materials and the intermediates, and the final products of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., such as from about 0° C. to about 125° C. and further such as at about room (or ambient) temperature, e.g., about 20° C.

Compounds of Formula (I) where G is NH, O, or S, R^{a} and R^{b} are independently alkoxy or hydroxy, and other groups are as defined in the Summary can be prepared as illustrated and described in Scheme 1 below.

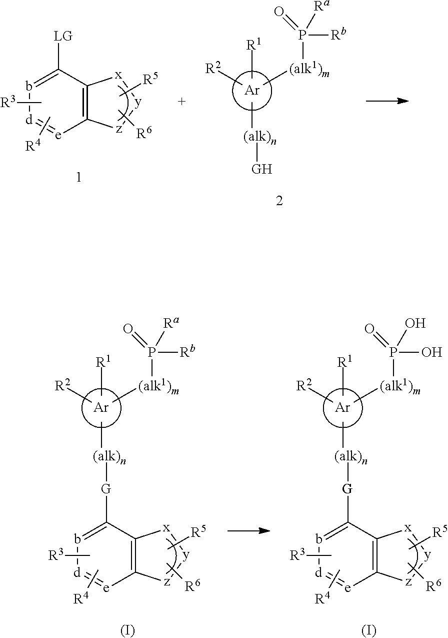

Treatment of a compound of formula 1, where LG is a suitable leaving group such as halo, triflate, and the like, and b, d, e, x, y, and z are as defined in the Summary and R³ and R⁴ are as defined in the Summary or a precursor group thereof (e.g., hydroxy is a precursor group of alkoxy, alkoxycarbonyl is a precursor group for aminocarbonyl), with a compound of the formula 2 where GH is N, O, or S, R^{a} and R^{b} are alkoxy, and Ar, alk, alk1, R¹, R², are as defined in the Summary or a precursor group thereof under SN2 reaction conditions provides a compound of Formula (I) where R^{a} and R^{b} are alkoxy. The reaction is carried out in the presence of a suitable organic or inorganic base such as potassium carbonate, cesium carbonate, triethylamine, DIEA, and the like, in a suitable organic solvent such as acetonitrile, DMSO, ethanol, and the like, either at room temperature or heating. Compounds of

Compounds of formula 1 and 2 are either commercially available or they can be prepared by methods well known the art. For example, synthesis of a number of compounds of formula 1 are described in Synthetic Examples below. Removal of the alkyl group from a compound of Formula (I) where R^{a} and R^{b} are alkoxy, provides a corresponding compound of Formula (I) where R^{a} and R^{b} are hydroxy.

In addition to above, compounds of Formula (I) can be converted to other compounds of Formula (I) by method well known in the art. Some such methods are described in Synthetic Examples below.

### Testing

The ENPP1 inhibitory activity of the compounds of the present disclosure can be tested using the in vitro assays described in Biological Examples 1 and 2 below.

### Administration and Pharmaceutical Composition

In general, the compounds of this disclosure will be administered in a therapeutically effective amount by any of the accepted modes of administration for agents that serve similar utilities. Therapeutically effective amounts of compounds this disclosure may range from about 0.01 to about 500 mg per kg patient body weight per day, which can be administered in single or multiple doses. A suitable dosage level may be from about 0.1 to about 250 mg/kg per day; about 0.5 to about 100 mg/kg per day. A suitable dosage level may be about 0.01 to about 250 mg/kg per day, about 0.05 to about 100 mg/kg per day, or about 0.1 to about 50 mg/kg per day. Within this range the dosage can be about 0.05 to about 0.5, about 0.5 to about 5 or about 5 to about 50 mg/kg per day. For oral administration, the compositions can be provided in the form of tablets containing about 1.0 to about 1000 milligrams of the active ingredient, particularly about 1, 5, 10, 15, 20, 25, 50, 75, 100, 150, 200, 250, 300, 400, 500, 600, 750, 800, 900, and 1000 milligrams of the active ingredient. The actual amount of the compound of this disclosure, i.e., the active ingredient, will depend upon numerous factors such as the severity of the disease to be treated, the age and relative health of the patient, the potency of the compound being utilized, the route and form of administration, and other factors.

In general, compounds of this disclosure will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen, which can be adjusted according to the degree of affliction. Compositions can take the form of tablets, pills, capsules, semisolids, powders, sustained release formulations, solutions, suspensions, elixirs, aerosols, or any other appropriate compositions.

The choice of formulation depends on various factors such as the mode of drug administration (e.g., for oral administration, formulations in the form of tablets, pills or capsules, including enteric coated or delayed release tablets, pills or capsules are preferred) and the bioavailability of the drug substance. Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a cross-linked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound of this disclosure in combination with at least one pharmaceutically acceptable excipient. Acceptable excipients are non-toxic, aid administration, and do not adversely affect the therapeutic benefit of the compound of this disclosure. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this disclosure in aerosol form. Inert gases suitable for this purpose are nitrogen, carbon dioxide, etc.

Other suitable pharmaceutical excipients and their formulations are described in Remington's Pharmaceutical Sciences, edited by E. W. Martin (Mack Publishing Company, 20th ed., 2000).

The level of the compound in a formulation can vary within the full range employed by those skilled in the art. Typically, the formulation will contain, on a weight percent (wt. %) basis, from about 0.01-99.99 wt. % of a compound of this disclosure based on the total formulation, with the balance being one or more suitable pharmaceutical excipients. For example, the compound is present at a level of about 1-80 wt. %.

The compounds of this disclosure may be used in combination with one or more other drugs in the treatment of diseases or conditions for which compounds of this disclosure or the other drugs may have utility. Such other drug(s) may be administered, by a route and in an amount commonly used therefore, contemporaneously or sequentially with a compound of the present disclosure. When a compound of this disclosure is used contemporaneously with one or more other drugs, a pharmaceutical composition in unit dosage form containing such other drugs and the compound of the present disclosure is preferred. However, the combination therapy may also include therapies in which the compound of this disclosure and one or more other drugs are administered on different overlapping schedules. It is also contemplated that when used in combination with one or more other active ingredients, the compounds of the present disclosure and the other active ingredients may be used in lower doses than when each is used singly. Accordingly, the pharmaceutical compositions of the present disclosure also include those that contain one or more other drugs, in addition to a compound of the present disclosure.

The above combinations include combinations of a compound of this disclosure not only with one other drug, but also with two or more other active drugs. Likewise, a compound of this disclosure may be used in combination with other drugs that are used in the prevention, treatment, control, amelioration, or reduction of risk of the diseases or conditions for which a compound of this disclosure is useful. Such other drugs may be administered, by a route and in an amount commonly used therefore, contemporaneously or sequentially with a compound of the present disclosure. When a compound of this disclosure is used contemporaneously with one or more other drugs, a pharmaceutical composition containing such other drugs in addition to the compound of this disclosure can be used. Accordingly, the pharmaceutical compositions of the present disclosure also include those that also contain one or more other active ingredients, in addition to a compound of this disclosure. The weight ratio of the compound of this disclosure to the second active ingredient may be varied and will depend upon the effective dose of each ingredient. Generally, an effective dose of each will be used.

Where the subject in need is suffering from or at risk of suffering from cancer, the subject can be treated with a compound of this disclosure in any combination with one or more other anti-cancer agents. In some embodiments, one or more of the anti-cancer agents are proapoptotic agents. Examples of anti-cancer agents include, but are not limited to, any of the following: gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec™), geldanamycin, 17-N-Allylamino-17-Demethoxygeldanamycin (17-AAG), flavopiridol, LY294002, bortezomib, trastuzumab, BAY 11-7082, PKC412, or PD184352, Taxol™, also referred to as “paclitaxel”, which is a well-known anti-cancer drug which acts by enhancing and stabilizing microtubule formation, and analogs of Taxol™, such as Taxotere™. Compounds that have the basic taxane skeleton as a common structure feature, have also been shown to have the ability to arrest cells in the G2-M phases due to stabilized microtubules and may be useful for treating cancer in combination with the compounds described herein.

Further examples of anti-cancer agents for use in combination with a compound of this disclosure include inhibitors of mitogen-activated protein kinase signaling, e.g., U0126, PD98059, PD184352, PD0325901, ARRY-142886, SB239063, SP600125, BAY 43-9006, wortmannin, or LY294002; Syk inhibitors; antibodies (e.g., rituxan); MET inhibitor such as foretinib, carbozantinib, or crizotinib; VEGFR inhibitor such as sunitinib, sorafenib, regorafinib, lenvatinib, vandetanib, carbozantinib, axitinib; EGFR inhibitor such as afatinib, brivanib, carbozatinib, erlotinib, gefitinib, neratinib, lapatinib; PI3K inhibitor such as XL147, XL765, BKM120 (buparlisib), GDC-0941, BYL719, IPI145, BAY80-6946. BEX235 (dactolisib), CAL101 (idelalisib), GSK2636771, TG100-115; MTOR inhibitor such as rapamycin (sirolimus), temsirolimus, everolimus, XL388, XL765, AZD2013, PF04691502, PKI-587, BEZ235, GDC0349; MEK inhibitor such as AZD6244, trametinib, PD184352, pimasertinib, GDC-0973, AZD8330; and proteasome inhibitor such as carfilzomib, MLN9708, delanzomib, or bortezomib.

Other anti-cancer agents that can be employed in combination with a compound of this disclosure include Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or Ril2), interferon alfa-2a; interferon alfa-2b; interferon alfa-n1; interferon alfa-n3; interferon beta-1a; interferon gamma-1 b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anti-cancer agents that can be employed in combination with a compound of the disclosure such as 8-(3-(4-acryloylpiperazin-1-yl)propyl)-6-(2,6-dichloro-3,5-dimethoxyphenyl)-2-(methylamino)pyrido[2,3-d]pyrimidin-7(8H)-one used to determine the anti-tumor activity in HGS and RT4 tumor models (Example 4 below: In HGS model, vehicle dosed group reached tumor size 645 dosing at day 42 after inoculation whereas for animals treated with 20/kg of compound, the tumor size was 55 mm3 showing significant antitumor activity and induced tumor regression), include: 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; Bfgf inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A; diethylstilbesterol; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras famesyl protein transferase inhibitors; ras inhibitors; ras-GAP inhibitor; retelliptine demethylated; rhenium Re 186 etidronate; rhizoxin; ribozymes; R.sub.11 retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

Yet other anticancer agents that can be employed in combination with a compound of this disclosure include alkylating agents, antimetabolites, natural products, or hormones, e.g., nitrogen mustards (e.g., mechloroethamine, cyclophosphamide, chlorambucil, etc.), alkyl sulfonates (e.g., busulfan), nitrosoureas (e.g., carmustine, lomusitne, etc.), or triazenes (decarbazine, etc.). Examples of antimetabolites include but are not limited to folic acid analog (e.g., methotrexate), or pyrimidine analogs (e.g., cytarabine), purine analogs (e.g., mercaptopurine, thioguanine, pentostatin).

Examples of natural products useful in combination with a compound of this disclosure include but are not limited to vinca alkaloids (e.g., vincristine), epipodophyllotoxins (e.g., etoposide), antibiotics (e.g., daunorubicin, doxorubicin, bleomycin), enzymes (e.g., L-asparaginase), or biological response modifiers (e.g., interferon alpha).

Examples of alkylating agents that can be employed in combination a compound of this disclosure) include, but are not limited to, nitrogen mustards (e.g., mechloroethamine, cyclophosphamide, chlorambucil, melphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, etc.). Examples of antimetabolites include, but are not limited to folic acid analog (e.g., methotrexate), or pyrimidine analogs (e.g., fluorouracil, floxuridine, cytarabine), purine analogs (e.g., mercaptopurine, thioguanine, pentostatin.

Examples of hormones and antagonists useful in combination a compound of this disclosure include, but are not limited to, adrenocorticosteroids (e.g., prednisone), progestins (e.g., hydroxyprogesterone caproate, megestrol acetate, medroxyprogesterone acetate), estrogens (e.g., diethylstilbestrol, ethinyl estradiol), antiestrogen (e.g., tamoxifen), androgens (e.g., testosterone propionate, fluoxymesterone), antiandrogen (e.g., flutamide), gonadotropin releasing hormone analog (e.g., leuprolide). Other agents that can be used in the methods and compositions described herein for the treatment or prevention of cancer include platinum coordination complexes (e.g., cisplatin, carboblatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide).

Examples of anti-cancer agents which act by arresting cells in the G2-M phases due to stabilized microtubules and which can be used in combination with an irreversible Btk inhibitor compound include without limitation the following marketed drugs and drugs in development: Erbulozole (also known as R-55104), Dolastatin 10 (also known as DLS-10 and NSC-376128), Mivobulin isethionate (also known as CI-980), Vincristine, NSC-639829, Discodermolide (also known as NVP-XX-A-296), ABT-751 (Abbott, also known as E-7010), Altorhyrtins (such as Altorhyrtin A and Altorhyrtin C), Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (also known as LU-103793 and NSC-D-669356), Epothilones (such as Epothilone A, Epothilone B, Epothilone C (also known as desoxyepothilone A or dEpoA), Epothilone D (also referred to as KOS-862, dEpoB, and desoxyepothilone B), Epothilone E, Epothilone F, Epothilone B N-oxide, Epothilone A N-oxide, 16-aza-epothilone B, 21-aminoepothilone B (also known as BMS-310705), 21-hydroxyepothilone D (also known as Desoxyepothilone F and dEpoF), 26-fluoroepothilone), Auristatin PE (also known as NSC-654663), Soblidotin (also known as TZT-1027), LS-4559-P (Pharmacia, also known as LS-4577), LS-4578 (Pharmacia, also known as LS-477-P), LS-4477 (Pharmacia), LS-4559 (Pharmacia), RPR-112378 (Aventis), Vincristine sulfate, DZ-3358 (Daiichi), FR-182877 (Fujisawa, also known as WS-9885B), GS-164 (Takeda), GS-198 (Takeda), KAR-2 (Hungarian Academy of Sciences), BSF-223651 (BASF, also known as ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (also known as LY-355703), AC-7739 (Ajinomoto, also known as AVE-8063A and CS-39·HCl), AC-7700 (Ajinomoto, also known as AVE-8062, AVE-8062A, CS-39-L-Ser·HCl, and RPR-258062A), Vitilevuamide, Tubulysin A, Canadensol, Centaureidin (also known as NSC-106969), T-138067 (Tularik, also known as T-67, TL-138067 and TI-138067), COBRA-1 (Parker Hughes Institute, also known as DDE-261 and WHI-261), H10 (Kansas State University), H16 (Kansas State University), Oncocidin A1 (also known as BTO-956 and DIME), DDE-313 (Parker Hughes Institute), Fijianolide B. Laulimalide, SPA-2 (Parker Hughes Institute), SPA-1 (Parker Hughes Institute, also known as SPIKET-P), 3-IAABU (Cytoskeleton/Mt. Sinai School of Medicine, also known as MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, also known as MF-191), TMPN (Arizona State University), Vanadocene acetylacetonate, T-138026 (Tularik), Monsatrol, Inanocine (also known as NSC-698666), 3-1AABE (Cytoskeleton/Mt. Sinai School of Medicine), A-204197 (Abbott), T-607 (Tuiarik, also known as T-900607), RPR-115781 (Aventis), Eleutherobins (such as Desmethyleleutherobin, Desaetyleleutherobin, Isoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (also known as NSCL-96F037), D-68838 (Asta Medica), D-68836 (Asta Medica), Myoseverin B, D-43411 (Zentaris, also known as D-81862), A-289099 (Abbott), A-318315 (Abbott), HTI-286 (also known as SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi).

Further examples of anti-cancer agents for use in combination with a compound of this disclosure include immune checkpoint inhibitors. Exemplary immune checkpoint inhibitors include inhibitors (smack molecules or biologics) against immune checkpoint molecules such as CD27, CD28, CD40, CD122, CD96, CD73, CD39, CD47, OX40, GITR, CSFIR, JAK, PI3K delta, PI3K gamma, TAM kinase, arginase, CD137 (also known as 4-1BB), ICOS, A2AR, A2BR, HIF-2α, B7-H3, B7-H4, BTLA, CTLA-4, LAG3, TIM3, VISTA, CD96, TIGIT, PD-1, PD-L1 and PD-L2. In some embodiments, the immune checkpoint molecule is a stimulatory checkpoint molecule selected from CD27, CD28, CD40, ICOS, OX40, GITR, CD137 and STING. In some embodiments, the immune checkpoint molecule is an inhibitory checkpoint molecule selected from B7-H3, B7-H4, BTLA, CTLA-4, IDO, TDO, Arginase, KIR, LAG3, PD-1, TIM3, CD96, TIGIT and VISTA. In some embodiments, the compounds provided herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the inhibitor of an immune checkpoint molecule is an inhibitor of PD-1, e.g., an anti-PD-1 monoclonal antibody. In some embodiments, the anti-PD-1 monoclonal antibody is nivolumab, pembrolizumab (also known as MK-3475), pidilizumab, SHR-1210, PDR001, or AMP-224. In some embodiments, the anti-PD-1 monoclonal antibody is nivolumab, or pembrolizumab or PDR001. In some embodiments, the anti-PD1 antibody is pembrolizumab.

In some embodiments, the inhibitor of an immune checkpoint molecule is an inhibitor of PD-L1, e.g., an anti-PD-L1 monoclonal antibody. In some embodiments, the anti-PD-L1 monoclonal antibody is BMS-935559, MEDI4736, MPDL3280A (also known as RG7446), or MSB0010718C. In some embodiments, the anti-PD-L1 monoclonal antibody is MPDL3280A (atezolizumab) or MEDI4736 (durvalumab).

In some embodiments, the inhibitor of an immune checkpoint molecule is an inhibitor of CTLA-4, e.g., an anti-CTLA-4 antibody. In some embodiments, the anti-CTLA-4 antibody is ipilimumab or tremelimumab. In some embodiments, the inhibitor of an immune checkpoint molecule is an inhibitor of LAG3, e.g., an anti-LAG3 antibody. In some embodiments, the anti-LAG3 antibody is BMS-986016 or LAG525. In some embodiments, the inhibitor of an immune checkpoint molecule is an inhibitor of GITR, e.g., an anti-GITR antibody. In some embodiments, the anti-GITR antibody is TRX518 or, MK-4166, INCAGN01876 or MK-1248. In some embodiments, the inhibitor of an immune checkpoint molecule is an inhibitor of OX40, e.g., an anti-OX40 antibody or OX40L fusion protein. In some embodiments, the anti-OX40 antibody is MED10562 or, INCAGN01949, GSK2831781, GSK-3174998, MOXR-0916, PF-04518600 or LAG525. In some embodiments, the OX40L fusion protein is MEDI6383

### EXAMPLES

The following preparations of compounds of Formula (I) are given to enable those skilled in the art to more clearly understand and to practice the present disclosure. They should not be considered as limiting the scope of the disclosure, but merely as being illustrative and representative thereof.

All solvents used were commercially available and were used without further purification. Reactions were typically run using anhydrous solvents under an inert atmosphere of nitrogen.

¹H spectra were recorded at 400 MHz or 300 MHz for proton on a Bruker 400 NMR Spectrometer equipped with a Bruker 400 BBO probe or Bruker BBFO ULTRASHIELD™ 300 AVANCE III, respectively. All deuterated solvents contained typically 0.03% to 0.05% v/v tetramethylsilane, which was used as the reference signal (set at S 0.00 for both ¹H and ¹³C).

LCMS analyses were performed on a SHIMADZU LCMS consisting of an UFLC 20-AD and LCMS 2020 MS detector. The Diode Array Detector was scanned from 190-400 nm. The mass spectrometer was equipped with an electrospray ion source (ESI) operated in a positive or negative mode. The mass spectrometer was scanned between m/z 90-900 with a scan time from 0.5 to 3.0 s.

HPLC analyses were performed on a SHIMADZU UFLC with two LC20 AD pump and a SPD-M20A Photodiiode Array Detector. The column used was an XBridge C18, 3.5 μm, 4.6×100 mm. A linear gradient was applied, starting at 90% A (A: 0.05% TFA in water) and ending at 95% B (B: 0.05% TFA in MCCN) over 10 min with a total run time of 15 min. The column temperature was at 40° C. with the flow rate of 1.5 mL/min. The Diode Array Detector was scanned from 200-400 nm.

Thin layer chromatography (TLC) was performed on Alugram® (Silica gel 60 F254) from Mancherey-Nagel and UV was typically used to visualize the spots. Additional visualization methods were also employed in some cases. In these cases the TLC plate was developed with iodine (generated by adding approximately 1 g of I₂ to 10 g silica gel and thoroughly mixing), ninhydrin (available commercially from Aldrich), or Magic Stain (generated by thoroughly mixing 25 g (NH₄)₆Mo₇O₂₄·4H₂O, 5 g (NH₄)₂Ce(IV)(NO₃)₆ in 450 mL water and 50 mL concentrated H₂SO₄) to visualize the compound. Flash chromatography was performed using 40-63 μm (230-400 mesh) silica gel from Silicycle following analogous techniques to those disclosed in Still, W. C.; Kahn, M.; and Mitra, M. Journal of Organic Chemistry, 1978, 43, 2923. Typical solvents used for flash chromatography or thin layer chromatography were mixtures of chloroform/methanol, dichloromethane/methanol, ethyl acetate/methanol and hexanes/ethyl acetate.

### SYNTHETIC EXAMPLES

### Example 1

### Synthesis of 4-[([1-methylpyrazolo[4,3-d]pyrimidin-7-yl]amino)methyl]phenyl-phosphonic acid

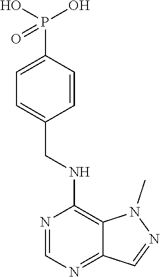 [SMILES: Cn1ncc2ncnc(NCc3ccc(P(=O)(O)O)cc3)c21]

### Step 1: diethyl 4-(aminomethyl)phenylphosphonate

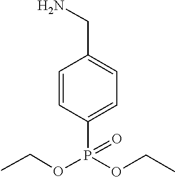 [SMILES: CCOP(=O)(OCC)c1ccc(CN)cc1]

To a mixture of 1-(4-iodophenyl)methanamine (500 mg, 2.15 mmol, 1.00 equiv) in water (10 mL) were added triethyl phosphate (1172 mg, 6.44 mmol, 3.00 equiv), PdCl₂ (38 mg, 0.22 mmol, 0.10 equiv), TBAB (692 mg, 2.15 mmol, 1.00 equiv) and DIEA (832 mg, 6.44 mmol, 3.00 equiv). After stirring for overnight at 100° C., the reaction mixture was cooled to room temperature and concentrated under reduced pressure. The residue was purified by column chromatography on silica gel 80 g (eluent: dichloromethane methyl alcohol 100%, 90:10) to give the title compound (375 mg, 72%) as a colorless oil.

### Step 2: diethyl 4-[([1-methylpyrazolo[4,3-d]pyrimidin-7-yl]amino)methyl]phenyl-phosphonate

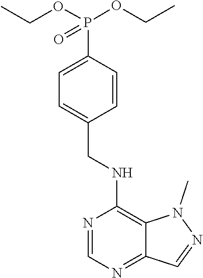

A mixture of 7-chloro-1-methylpyrazolo[4,3-d]pyrimidine (160 mg, 0.95 mmol, 1.00 equiv), TEA (288 mg, 2.847 mmol, 3.00 equiv) and diethyl 4-(aminomethyl)phenylphosphonate (346 mg, 1.42 mmol, 1.50 equiv) in EtOH (8 mL) was stirred overnight at 80° C. After cooling down to room temperature, the reaction mixture was concentrated under reduced pressure. The residue was purified by column chromatography on silica gel 40 g (eluent: petroleum ether ethyl acetate 100%, 50:50) to give the title compound (260 mg, 73%) as an off-white solid.

### Step 3: 4-[([1-methylpyrazolo[4,3-d]pyrimidin-7-yl]amino)methyl]phenylphosphonic acid

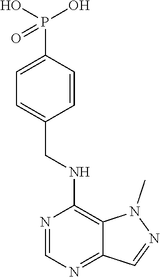 [SMILES: Cn1ncc2ncnc(NCc3ccc(P(=O)(O)O)cc3)c21]

To a stirred solution of diethyl 4-[([1-methylpyrazolo[4,3-d]pyrimidin-7-yl]amino)-methyl]phenylphosphonate (240 mg, 0.64 mmol, 1.00 equiv) in DCM (6 mL) was added bromotrimethylsilane (2 mL) at room temperature. After stirring for 12 h at room temperature, the resulting mixture was concentrated under reduced pressure. The crude product was purified by prep-HPLC with the following conditions (Column: Sunfire prep C18 column, 30*150, Sum; Mobile Phase A: Water (0.1% FA), Mobile Phase B: ACN; Flow rate: 60 mL/min; Gradient: 2 B to 10 B in 7 min; 254/220 nm) to afford the title compound (89.8 mg, 44%) as a white solid. MS (ESI, pos. ion) m/z: 320.2 (M+1). ¹H NMR (300 MHz, DMSO-d₆. ppm) δ 8.16 (d, J=10.3 Hz, 1H), 7.97 (d, J=11.9 Hz, 2H), 7.62 (dd, J=12.7, 7.9 Hz, 2H), 7.46 (dd, J=8.1, 3.4 Hz, 2H), 4.80 (d, J=5.7 Hz, 2H), 4.33 (s, 3H).

### Example 2

### Synthesis of ethoxy (4-([[7-(methylcarbamoyl)-5H-pyrrolo[3,2-d]pyrimidin-4-yl]amino]methyl)phenyl)phosphinic acid

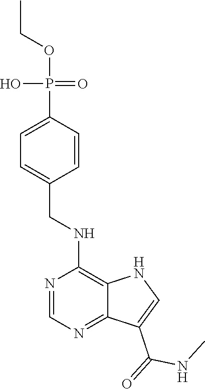 [SMILES: CCOP(=O)(O)c1ccc(CNc2ncnc3c(C(=O)NC)c[nH]c23)cc1]

### Step 1: ethyl 4-([[4-(diethoxyphosphoryl)phenyl]methyl]amino)-5H-pyrrolo[3,2-d]-pyrimidine-7-carboxylate

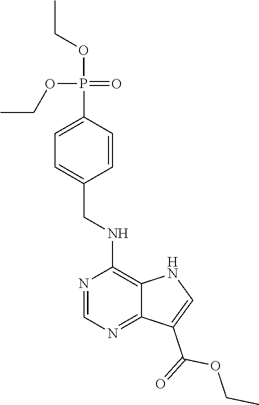 [SMILES: CCOC(=O)c1c[nH]c2c(NCc3ccc(P(=O)(OCC)OCC)cc3)ncnc12]

The title compound was synthesized by the same method as described in Example 1, step 2 except ethyl 4-chloro-5H-pyrrolo[3,2-d]pyrimidine-7-carboxylate (300 mg, 1.33 mmol) was used. The crude product was purified by column chromatography on silica gel 80 g (eluent: dichloromethane methyl alcohol 100%, 95:5) to give the title compound (500 mg, 85%) as a white solid.

### Step 2: ethoxy(4-([[7-(methylcarbamoyl)-5H-pyrrolo[3,2-d]pyrimidin-4-yl]amino]methyl)-phenyl)phosphinic acid

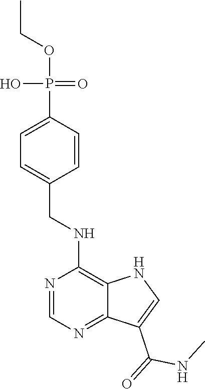 [SMILES: CCOP(=O)(O)c1ccc(CNc2ncnc3c(C(=O)NC)c[nH]c23)cc1]

A mixture of ethyl 4-([[4-(diethoxyphosphoryl)phenyl]methyl]amino)-5H-pyrrolo[3,2-d]-pyrimidine-7-carboxylate (500 mg, 1.16 mmol) in CH₃NH₂ (30% in MeOH) was stirred overnight at 80° C. After cooling to room temperature, the reaction mixture was concentrated and the crude product was purified by column chromatography on silica gel 80 g (eluent: dichloromethane methyl alcohol 100%, 60:40) to give the title compound (340 mg, 74%) as a white solid. MS (ESI, pos. ion) m/z: 390.3 (M+1). ¹H NMR (300 MHz, DMSO-d₆. ppm) 12.10 (s, 1H), δ 8.39 (d, J=23.7 Hz, 2H), 8.04 (s, 1H), 7.61 (m, 2H), 7.43 (s, 2H), 4.82 (d, J=5.4 Hz, 2H), 3.82 (m, 2H), 3.17 (s, 1H), 2.87 (d, J=4.7 Hz, 3H), 1.15 (t, J=7.0 Hz, 3H).

### Example 3

### Synthesis of 4-([[7-(methylcarbamoyl)-5H-pyrrolo[3,2-d]pyrimidin-4-yl]amino]-methyl)phenylphosphonic acid

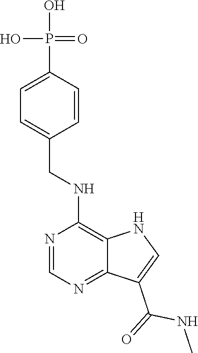 [SMILES: CNC(=O)c1c[nH]c2c(NCc3ccc(P(=O)(O)O)cc3)ncnc12]

The title compound was synthesized by the same method as described in Example 1, step 3 except ethoxy(4-([[7-(methylcarbamoyl)-5H-pyrrolo[3,2-d]-pyrimidin-4-yl]amino]methyl)-phenyl)phosphinic acid (290 mg, 0.75 mmol) was used. The crude product was purified by prep-HPLC with the following conditions (Column: Xselect CSH OBD Column 30*150 mm Sum, n; Mobile Phase A: Water (0.1% FA), Mobile Phase B: ACN; Flow rate: 60 mL/min; Gradient: 2 B to 15 B in 7 min, 254; 220 nm) to afford the title compound (41.2 mg, 15%) as a white solid. MS (ESI, pos. ion) m/z: 362.2 (M+1). ¹H NMR (300 MHz, DMSO-d₆ and a drop of 1 M HCl. ppm) δ 13.24 (d, J=3.3 Hz, 1H), 10.42 (t, J=6.0 Hz, 1H), 8.64 (m, 1H), 8.52 (s, 1H), 8.37 (d, J=3.1 Hz, 1H), 7.65 (dd, J=12.7, 7.8 Hz, 2H), 7.48 (dd, J=8.0, 3.3 Hz, 2H), 4.93 (d, J=5.8 Hz, 2H), 2.81 (d, J=3.9 Hz, 3H).

### Example 4

### Synthesis of ethoxy([4-([[7-(methylcarbamoyl)-5H-pyrrolo[3,2-d]pyrimidin-4-yl]amino]-methyl)-phenyl]methyl)phosphinic acid

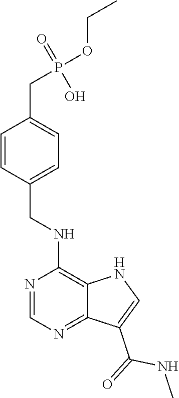 [SMILES: CCOP(=O)(O)Cc1ccc(CNc2ncnc3c(C(=O)NC)c[nH]c23)cc1]

### Step 1: diethyl (4-(aminomethyl)benzyl)phosphonate

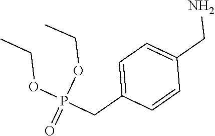 [SMILES: CCOP(=O)(Cc1ccc(CN)cc1)OCC]

The title compound was synthesized by the same method as described in Example 1, step 1 except tert-butyl N-[[4-(bromomethyl)phenyl]methyl]carbamate (2 g, 6.66 mmol) was used. The title compound (1.8 g, 55%) was obtained as a brown oil.

### Step 2: ethyl 4-[([4-[(diethoxyphosphoryl)methyl]phenyl]methyl)amino]-5H-pyrrolo[3,2-d]-pyrimidine-7-carboxylate

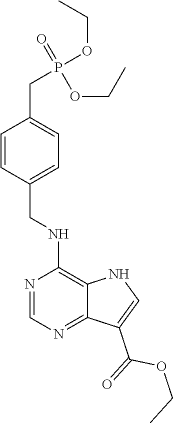 [SMILES: CCOC(=O)c1c[nH]c2c(NCc3ccc(CP(=O)(OCC)OCC)cc3)ncnc12]

The title compound was synthesized by the same method as described in Example 1, step 2 except ethyl 4-chloro-5H-pyrrolo[3,2-d]pyrimidine-7-carboxylate (300 mg, 1.33 mmol) and diethyl [4-(aminomethyl)phenyl]methylphosphonate (684 mg, 2.66 mmol) were used. The title compound (256 mg, 41%) was obtained as a white solid.

### Step 3: ethoxy([4-([[7-(methylcarbamoyl)-5H-pyrrolo[3,2-d]pyrimidin-4-yl]amino]methyl)-phenyl]methyl)phosphinic acid

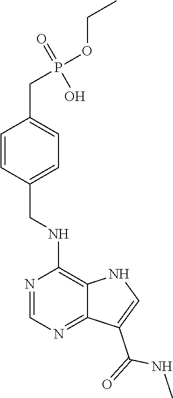 [SMILES: CCOP(=O)(O)Cc1ccc(CNc2ncnc3c(C(=O)NC)c[nH]c23)cc1]

The title compound was synthesized by the same method as described in Example 2, step 2 except ethyl 4-[([4-[(diethoxyphosphoryl)methyl]phenyl]methyl)amino]-5H-pyrrolo[3,2-d]pyrimidine-7-carboxylate (256 mg, 0.57 mmol) was used. The title compound (171 mg, 73%) was obtained as a white solid. MS (ESI, pos. ion) m/z: 404.0 (M+1).

### Example 5

### Synthesis of [4-([[7-(methylcarbamoyl)-5H-pyrrolo[3,2-d]pyrimidin-4-yl]amino]-methyl)phenyl]methylphosphonic acid

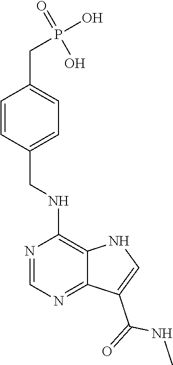 [SMILES: CNC(=O)c1c[nH]c2c(NCc3ccc(CP(=O)(O)O)cc3)ncnc12]

The title compound was synthesized by the same method as described in Example 1, step 3 except ethoxy([4-([[7-(methylcarbamoyl)-5H-pyrrolo[3,2-d]pyrimidin-4-yl]amino]methyl)phenyl]methyl)phosphinic acid (171 mg, 0.424 mmol) was used. The crude product was purified by prep-HPLC with the following conditions (Column: Sunfire prep C18 column, 30*150, 5 um; Mobile Phase A: Water (0.1% FA), Mobile Phase B: ACN; Flow rate: 60 mL/min; Gradient: 5 B to 15 B in 7 min; 254/220 nm) to afford the title compound (54.5 mg, 34%) as a white solid. MS (ESI, pos. ion) m/z: 376.2 (M+1). ¹H NMR (300 MHz, DMSO-d₆, ppm) δ 11.57 (s, 1H), 8.42 (d, J=5.1 Hz, 1H), 8.34 (s, 1H), 7.97 (s, 1H), 7.82 (s, 1H), 7.36-7.13 (m, 4H), 4.69 (d, J=4.9 Hz, 2H), 3.02-2.76 (m, 5H).

### Example 6

### Synthesis of ethoxy(2-[1-[7-(methylcarbamoyl)-5H-pyrrolo[3,2-d]pyrimidin-4-yl]piperidin-4-yl]ethyl)phosphinic acid

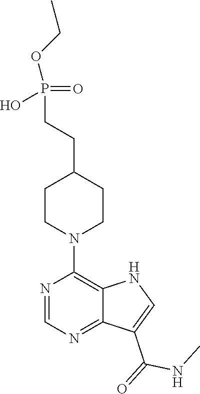 [SMILES: CCOP(=O)(O)CCC1CCN(c2ncnc3c(C(=O)NC)c[nH]c23)CC1]

### Step 1: ethyl 4-[4-[2-(diethoxyphosphoryl)ethyl]piperidin-1-yl]-5H-pyrrolo[3,2-d]-pyrimidine-7-carboxylate

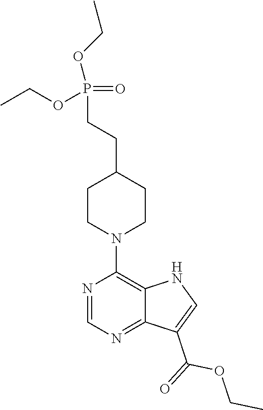 [SMILES: CCOC(=O)c1c[nH]c2c(N3CCC(CCP(=O)(OCC)OCC)CC3)ncnc12]

The title compound was synthesized by the same method as described in Example 1, step 2 except ethyl 4-chloro-5H-pyrrolo[3,2-d]pyrimidine-7-carboxylate (350 mg, 1.55 mmol) and diethyl 2-(piperidin-4-yl)ethylphosphonate (773 mg, 3.10 mmol) were used. The title compound (633 mg, 92%) was obtained as a yellow oil.

### Step 2: ethoxy(2-[1-[7-(methylcarbamoyl)-5H-pyrrolo[3,2-d]pyrimidin-4-yl]piperidin-4-yl]ethyl)phosphinic acid

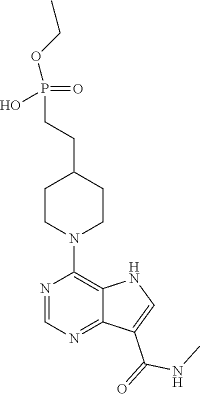 [SMILES: CCOP(=O)(O)CCC1CCN(c2ncnc3c(C(=O)NC)c[nH]c23)CC1]

The title compound was synthesized by the same method as described in Example 2, step 2 except ethyl 4-[4-[2-(diethoxyphosphoryl)ethyl]piperidin-1-yl]-5H-pyrrolo[3,2-d]pyrimidine-7-carboxylate (300 mg, 0.68 mmol) was used. The crude product was purified by prep-HPLC with the following conditions (Column: XSelect CSH Fluoro Phenyl, 30 mm×150 mm, 5 um; Mobile Phase A: Water (0.1% FA), Mobile Phase B: ACN; Flow rate: 60 mL/min; Gradient: 3 B to 30 B in 10 min, 254/220 nm) to afford the title compound (225 mg, 82%) as a white solid. MS (ESI, pos. ion) m/z: 396.3 (M+1). ¹H NMR (300 MHz, DMSO-d₆ and D₂O, ppm) δ 8.31 (s, 1H), 7.98 (s, 1H), 4.46 (d, J=13.1 Hz, 2H), 3.83 (m, 2H), 3.19-3.04 (m, 2H), 2.83 (s, 3H), 1.86-1.75 (m, 2H), 1.70-1.30 (m, 5H), 1.13 (m, 5H).

### Example 7

### Synthesis of 2-[1-[7-(methylcarbamoyl)-5H-pyrrolo[3,2-d]pyrimidin-4-yl]piperidin-4-yl]ethylphosphonic acid

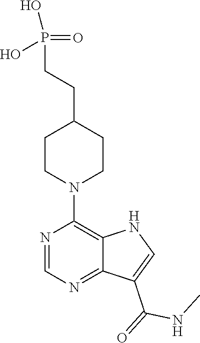 [SMILES: CNC(=O)c1c[nH]c2c(N3CCC(CCP(=O)(O)O)CC3)ncnc12]

The title compound was synthesized by the method as described in Example 1, step 3 except ethoxy(2-[1-[7-(methylcarbamoyl)-5H-pyrrolo[3,2-d]pyrimidin-4-yl]piperidin-4-yl]ethyl)phosphinic acid (225 mg, 0.57 mmol) was used. The crude product was purified by prep-HPLC with the following conditions (Column: XSelect CSH Fluoro Phenyl, 30 mm×150 mm, Sum; Mobile Phase A: Water (0.1% FA), Mobile Phase B: ACN; Flow rate: 60 mL/min; Gradient: 3 B to 30 B in 10 min, 254/220 nm) to afford the title compound (68.1 mg, 32%) as a white solid. MS (ESI, pos. ion) m/z: 368.3 (M+1). ¹H NMR (300 MHz, DMSO-d₆ and D20, ppm) δ 8.42 (d, J=2.1 Hz, 1H), 8.28-8.20 (m, 1H), 4.65 (s, 2H), 3.33 (s, 2H), 2.79 (d, J=1.6 Hz, 3H), 1.90 (d, J=13.0 Hz, 2H), 1.72 (s, 1H), 1.67-1.49 (m, 1H), 1.48-1.40 (m, 3H), 1.20 (d, J=12.4 Hz, 2H).

### Example 8

### Synthesis of diethyl [1-[7-(methylcarbamoyl)-5H-pyrrolo[3,2-d]pyrimidin-4-yl]piperidin-4-yl]methylphosphonate and ethoxy([1-[7-(methylcarbamoyl)-5H-pyrrolo[3,2-d]pyrimidin-4-yl]piperidin-4-yl]methyl)phosphinic acid

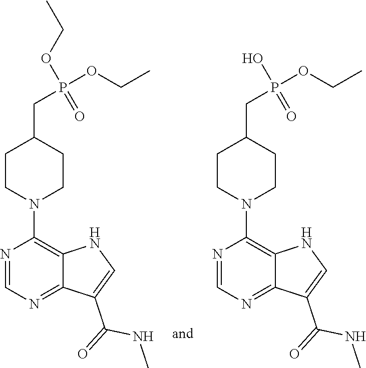 [SMILES: CCOP(=O)(CC1CCN(c2ncnc3c(C(=O)NC)c[nH]c23)CC1)OCC ; CCOP(=O)(O)CC1CCN(c2ncnc3c(C(=O)NC)c[nH]c23)CC1]

### Step 1: diethyl (1-benzylpiperidin-4-ylidene)methylphosphonate

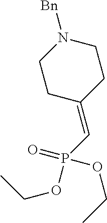 [SMILES: CCOP(=O)(C=C1CCN(Cc2ccccc2)CC1)OCC]

To a solution of 1-benzylpiperidin-4-one (1.50 g, 7.926 mmol, 1.00 equiv) in toluene (50.00 mL) were added NaH (0.63 g, 15.852 mmol, 2.00 equiv, 60%) and diethyl (diethoxyphosphoryl)methylphosphonate (2.74 g, 9.511 mmol, 1.20 equiv) at 0° C. under nitrogen atmosphere. After stirring for 2 h at 0° C., the reaction mixture was quenched with 2M HCl (15 mL). The resulting mixture was concentrated under reduced pressure and purified by silica gel column chromatography, eluted with EA/PE (1:1) to afford the title compound (1.8 g, 70%) as a colorless oil.

### Step 2: diethyl piperidin-4-ylmethylphosphonate

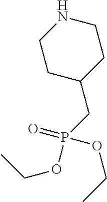

To a solution of diethyl (1-benzylpiperidin-4-ylidene)methylphosphonate (1.80 g, 5.566 mmol, 1.00 equiv) in MeOH (30.00 mL) was added Pd/C (0.20 g, 0.188 mmol, 10%). After stirring for 3 h under hydrogen atmosphere, the resulting mixture was filtered through a Celite. The filter cake was washed with MeOH. The filtrate was concentrated under reduced pressure to give the title compound (1.0 g, 76%) as a colorless oil.

### Step 3: ethyl 4-[4-[(diethoxyphosphoryl)methyl]piperidin-1-yl]-5H-pyrrolo[3,2-d]-pyrimidine-7-carboxylate

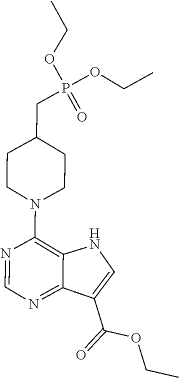 [SMILES: CCOC(=O)c1c[nH]c2c(N3CCC(CP(=O)(OCC)OCC)CC3)ncnc12]

The title compound was synthesized by the method as described in Example 1, step 2 except diethylpiperidin-4-ylmethylphosphonate (400 mg, 1.700 mmol) and ethyl 4-chloro-5H-pyrrolo[3,2-d]pyrimidine-7-carboxylate (384 mg, 1.700 mmol) were used. The title compound (512 mg, 71%) was obtained as an off-white solid.

### Step 4: diethyl [1-[7-(methylcarbamoyl)-5H-pyrrolo[3,2-d]pyrimidin-4-yl]piperidin-4-yl]-methylphosphonate and ethoxy([1-[7-(methylcarbamoyl)-5H-pyrrolo[3,2-d]pyrimidin-4-yl]piperidin-4-yl]methyl)phosphinic acid

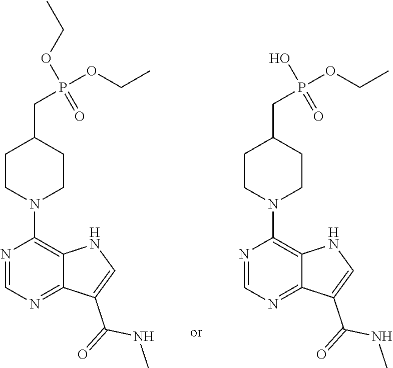 [SMILES: CCOP(=O)(CC1CCN(c2ncnc3c(C(=O)NC)c[nH]c23)CC1)OCC ; CCOP(=O)(O)CC1CCN(c2ncnc3c(C(=O)NC)c[nH]c23)CC1]

The title compounds was synthesized by the method as described in Example 2, step 2 except ethyl4-[4-[(diethoxyphosphoryl)methyl]piperidin-1-yl]-5H-pyrrolo[3,2-d]pyrimidine-7-carboxylate (512 mg, 1.21 mmol) was used. The crude product was purified by prep-HPLC Column: Xselect CSH OBD Column 30*150 mm 5 um, n; Mobile Phase A:Water (0.1% FA), Mobile Phase B: ACN; Flow rate: 60 mL/min; Gradient: 2 B to 10 B in 8 min; 254; 220 nm). The fractions containing the desired products were combined and lyophilized to give two fractions:

Fraction 1: Rt: 7.50 min. diethyl [1-[7-(methylcarbamoyl)-5H-pyrrolo[3,2-d]pyrimidin-4-yl]piperidin-4-yl]methylphosphonate (31.0 mg) was obtained as a colorless oil. MS (ESI, pos. ion) m/z: 410.3 (M+1). ¹H NMR (300 MHz, DMSO-d₆. ppm) δ 11.93 (s, 1H), 8.63 (q, J=4.7 Hz, 1H), 8.34 (s, 1H), 7.96 (s, 1H), 4.48 (d, J=13.2 Hz, 2H), 4.09-3.90 (m, 4H), 3.21-3.04 (m, 2H), 2.89 (d, J=4.7 Hz, 3H), 1.93 (d, J=12.6 Hz, 3H), 1.76 (dd, J=18.0, 6.3 Hz, 2H), 1.24 (t, J=7.1 Hz, 8H).

Fraction 2: Rt: 7.78 min. ethoxy([1-[7-(methylcarbamoyl)-5H-pyrrolo[3,2-d]pyrimidin-4-yl]piperidin-4-yl]methyl)phosphinic acid (30.3 mg) was obtained as a white solid. MS (ESI, pos. ion) m/z: 382.3 (M+1). ¹H NMR (300 MHz, DMSO-d₆. ppm) δ 11.98 (s, 1H), 8.63 (d, J=5.2 Hz, 1H), 8.35 (s, 1H), 7.96 (s, 1H), 4.48 (d, J=13.2 Hz, 2H), 3.93 (p, J=7.2 Hz, 2H), 3.11 (t, J=12.5 Hz, 2H), 2.88 (d, J=4.8 Hz, 3H), 1.96 (d, J=12.2 Hz, 3H), 1.66 (d, J=6.1 Hz, 1H), 1.61 (d, J=6.2 Hz, 1H), 1.33 (t, J=11.8 Hz, 2H), 1.22 (t, J=7.0 Hz, 3H).

### Example 9

### Synthesis of [1-[7-(methylcarbamoyl)-5H-pyrrolo[3,2-d]pyrimidin-4-yl]piperidin-4-yl]-methylphosphonic acid

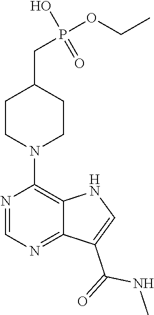 [SMILES: CCOP(=O)(O)CC1CCN(c2ncnc3c(C(=O)NC)c[nH]c23)CC1]

The title compound was synthesized by the method as described in Example 1, step 3 except ethoxy([1-[7-(methylcarbamoyl)-5H-pyrrolo[3,2-d]pyrimidin-4-yl]piperidin-4-yl]methyl)phosphinic acid (250 mg, 0.656 mmol) was used. The crude product was purified by prep-HPLC with the following conditions (Column: Xselect CSH OBD Column 30*150 mm 5 um, n; Mobile Phase A:Water (0.1% FA), Mobile Phase B: ACN; Flow rate: 60 mL/min; Gradient: 2 B to 10 B in 8 min, 254; 220 nm) to afford the title compound (121.8 mg, 52.6%) as a white solid. MS (ESI, pos. ion) m/z: 354.2 (M+1). ¹H NMR (300 MHz, DMSO-d₆, ppm) δ 12.01 (s, 1H), 8.62 (d, J=5.0 Hz, 1H), 8.35 (s, 1H), 7.97 (s, 1H), 4.49 (d, J=13.2 Hz, 2H), 3.11 (t, J=12.5 Hz, 2H), 2.88 (d, J=4.7 Hz, 3H), 1.99 (d, J=12.7 Hz, 3H), 1.58 (d, J=6.1 Hz, 1H), 1.52 (d, J=6.1 Hz, 1H), 1.34 (d, J=12.1 Hz, 1H), 1.26 (d, J=12.4 Hz, 1H).

### Example 10

### Synthesis of ethoxy(3-[1-[7-(methylcarbamoyl)-5H-pyrrolo[3,2-d]pyrimidin-4-yl]-piperidin-4-yl]propyl)phosphinic acid

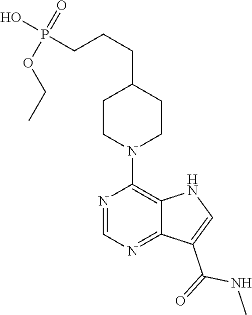 [SMILES: CCOP(=O)(O)CCCC1CCN(c2ncnc3c(C(=O)NC)c[nH]c23)CC1]

### Step 1: tert-butyl 4-[(2E)-3-(diethoxyphosphoryl)prop-2-en-1-yl]piperidine-1-carboxylate

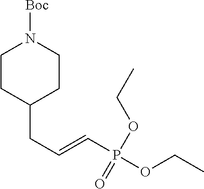 [SMILES: CCOP(=O)(/C=C/CC1CCN(C(=O)OC(C)(C)C)CC1)OCC]

The title compound was synthesized by the same method as described in Example 8, step 1 except tert-butyl 4-(2-oxoethyl)piperidine-1-carboxylate (2.00 g, 8.80 mmol) was used. The residue was purified by column chromatography on silica gel 120 g (eluent: petroleum ether ethyl acetate 100%, 50:50) to give the title compound (857 mg, 27%) as a yellow oil.

### Step 2: tert-butyl 4-[3-(diethoxyphosphoryl)propyl]piperidine-1-carboxylate

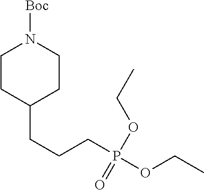 [SMILES: CCOP(=O)(CCCC1CCN(C(=O)OC(C)(C)C)CC1)OCC]

The title compound was synthesized by the same method as described in Example 8, step 2 except tert-butyl 4-[(2E)-3-(diethoxyphosphoryl)prop-2-en-1-yl]piperidine-1-carboxylate (857 mg, 2.37 mmol) was used. The title compound (850 mg, 99%) was obtained as a colorless oil.

### Step 3: diethyl (3-(piperidin-4-yl)propyl)phosphonate hydrochloride

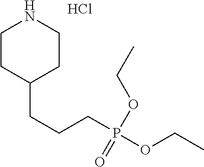 [SMILES: Cl ; CCOP(=O)(CCCC1CCNCC1)OCC]

To a stirred solution of tert-butyl 4-[3-(diethoxyphosphoryl)propyl]piperidine-1-carboxylate (850 mg, 2.34 mmol, 1.00 equiv) in EA (10 mL) was added 5 mL HCl (g, 2 M in EA) at room temperature. After stirring for 12 h at room temperature, the mixture was concentrated under reduced pressure to afford the title compound (610 mg, 98%) as a white solid.

### Step 4: Synthesis of ethyl 4-[4-[3-(diethoxyphosphoryl)propyl]piperidin-1-yl]-5H-pyrrolo-[3,2-d]pyrimidine-7-carboxylate

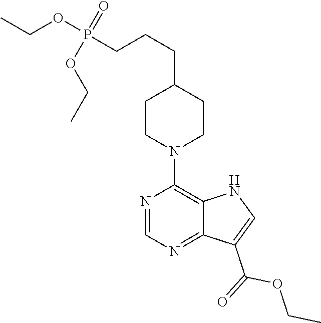 [SMILES: CCOC(=O)c1c[nH]c2c(N3CCC(CCCP(=O)(OCC)OCC)CC3)ncnc12]

The title compound was synthesized by the same method as described in Example 1, step 2 except diethyl (3-(piperidin-4-yl)propyl)phosphonate hydrochloride (610 mg, 2.04 mmol) was used. The residue was purified by column chromatography on silica gel 120 g (eluent: dichloromethane methyl alcohol 100%, 82:18) to give the title compound (749 mg, 62%) as a yellow oil. MS (ESI, pos. ion) m/z: 453.4 (M+1).

### Step 5: Synthesis of ethoxy(3-[1-[7-(methylcarbamoyl)-5H-pyrrolo[3,2-d]pyrimidin-4-yl]-piperidin-4-yl]propyl)phosphinic acid

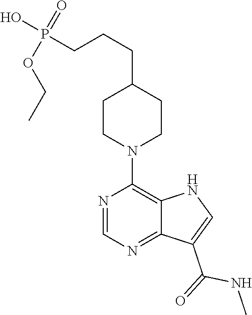 [SMILES: CCOP(=O)(O)CCCC1CCN(c2ncnc3c(C(=O)NC)c[nH]c23)CC1]

The title compound was synthesized by the same method as described in Example 2, step 2 except ethyl 4-[4-[3-(diethoxyphosphoryl)propyl]piperidin-1-yl]-5H-pyrrolo[3,2-d]pyrimidine-7-carboxylate (749 mg, 1.66 mmol) was used. The crude product was purified by prep-HPLC with the following conditions (Column: Xselect CSH OBD Column 30*150 mm 5 um, n; Mobile Phase A: Water (0.1% FA), Mobile Phase B: ACN; Flow rate: 60 mL/min; Gradient: 4 B to 15 B in 7 min, 15 B to 27 B in 10 min, 254/220 nm) to afford the title compound (585 mg, 86%) as a white solid. MS (ESI, pos. ion) m/z: 410.3 (M+1). ¹H NMR (300 MHz, DMSO-d₆, ppm) δ 12.00 (s, 1H), 8.63 (d, J=4.7 Hz, 1H), 8.34 (s, 1H), 7.96 (s, 1H), 4.51 (d, J=13.0 Hz, 2H), 3.92 (q, J=7.3 Hz, 2H), 3.20-3.00 (m, 2H), 2.88 (m, J=4.7 Hz, 4H), 1.77 (d, J=12.6 Hz, 2H), 1.56 (d, J=14.1 Hz, 6H), 1.31 (s, 1H), 1.20 (t, J=7.1 Hz, 4H)

### Example 11

### Synthesis of 3-[1-[7-(methylcarbamoyl)-5H-pyrrolo[3,2-d]pyrimidin-4-yl]piperidin-4-yl]propylphosphonic add

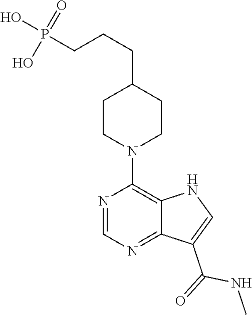 [SMILES: CNC(=O)c1c[nH]c2c(N3CCC(CCCP(=O)(O)O)CC3)ncnc12]

The title compound was synthesized by the same method as described in Example 1, step 3 except ethoxy(3-[1-[7-(methylcarbamoyl)-5H-pyrrolo[3,2-d]pyrimidin-4-yl]piperidin-4-yl]propyl)phosphinic acid (245 mg, 0.60 mmol) was used. The crude product was purified by prep-HPLC with the following conditions (Column: Xselect CSH OBD Column 30*150 mm 5 um, n; Mobile Phase A:Water (0.1% FA), Mobile Phase B: ACN; Flow rate: 60 mL/min; Gradient: 4 B to 15 B in 10 min, 254; 220 nm) to afford the title compound (93.6 mg, 41%) as a white solid. MS (ESI, pos. ion) m/z: 382.3 (M+1). ¹H NMR (400 MHz, DMSO-d₆, ppm) δ 11.96 (s, 1H), 8.59 (t, J=4.9 Hz, 1H), 8.32 (s, 1H), 7.94 (s, 1H), 4.49 (d, J=13.1 Hz, 2H), 3.06 (t, J=12.2 Hz, 2H), 2.86 (d, J=4.8 Hz, 3H), 1.75 (d, J=12.8 Hz, 2H), 1.62-1.50 (m, 1H), 1.48 (m, J=9.6 Hz, 4H), 1.29 (d, J=7.1 Hz, 2H), 1.24-1.06 (m, 2H).

### Example 12

### Synthesis of 4-([1H-pyrazolo[4,3-d]pyrimidin-7-ylamino]methyl)phenyl-phosphonic acid

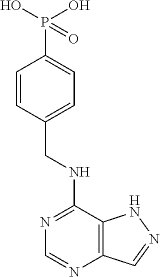 [SMILES: O=P(O)(O)c1ccc(CNc2ncnc3cn[nH]c23)cc1]

### Step 1: diethyl 4-([1H-pyrazolo[4,3-d]pyrimidin-7-ylamino]methyl)phenylphosphonate

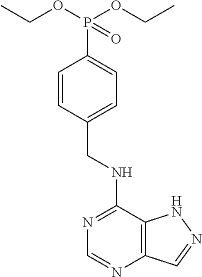 [SMILES: CCOP(=O)(OCC)c1ccc(CNc2ncnc3cn[nH]c23)cc1]

The title compound was synthesized by the same method as described in Example 1, step 2 except 7-chloro-1H-pyrazolo[4,3-d]pyrimidine (200 mg, 1.29 mmol) and diethyl 4-(aminomethyl)phenylphosphonate (378 mg, 1.55 mmol) were used. The title compound (220 mg, 47%) as a yellow solid was obtained. MS (ESI, pos. ion) m/z: 362.0 (M+1).

### Step 2: 4-([1H-pyrazolo[4,3-d]pyrimidin-7-ylamino]methyl)phenylphosphonic acid

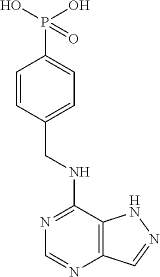 [SMILES: O=P(O)(O)c1ccc(CNc2ncnc3cn[nH]c23)cc1]

The title compound was synthesized by the same method as described in Example 1, step 3 except diethyl4-([1H-pyrazolo[4,3-d]pyrimidin-7-ylamino]methyl)phenylphosphonate (200 mg, 0.55 mmol) was used. The crude product was purified by prep-HPLC with the following conditions (Column: Xselect CSH OBD Column 30*150 mm 5 um, n; Mobile Phase A: Water (0.1% FA), Mobile Phase B: MeOH—Preparative; Flow rate: 60 mL/min; Gradient: 2 B to 25 B in 7 min, 254/220 nm) to afford the title compound (17.2 mg, 10%) as a white solid. MS (ESI, pos. ion) m/z: 306.0 (M+1). ¹H NMR (300 MHz, DMSO-d₆, ppm) δ 8.76 (s, 1H), 8.19 (m, 2H), 7.64 (dd, J=12.8, 7.9 Hz, 2H), 7.42 (dd, J=8.2, 3.3 Hz, 2H), 4.78 (d, J=5.9 Hz, 2H)

### Example 13

### Synthesis of ethoxy(4-([[3-(methylcarbamoyl)-1H-pyrazolo[4,3-d]pyrimidin-7-yl]amino]methyl)-phenyl)phosphinic acid

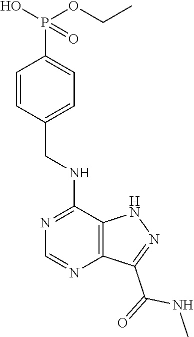 [SMILES: CCOP(=O)(O)c1ccc(CNc2ncnc3c(C(=O)NC)n[nH]c23)cc1]

### Step 1: 1-tert-butyl 3,5-dimethyl 4-nitropyrazole-1,3,5-tricarboxylate

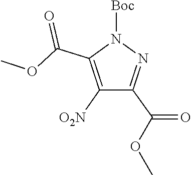 [SMILES: COC(=O)c1nn(C(=O)OC(C)(C)C)c(C(=O)OC)c1[N+](=O)[O-]]

To a solution of 3,5-dimethyl 4-nitro-1H-pyrazole-3,5-dicarboxylate (3.00 g, 13.09 mmol, 1.00 equiv) in THF (20 mL) were added TEA (3.97 g, 39.23 mmol, 3.00 equiv) and (Boc)₂O (6.86 g, 31.43 mmol, 2.40 equiv) at room temperature. After stirring overnight at 40° C., the reaction mixture was cooled to room temperature and concentrated under reduced pressure. The residue was purified by column chromatography on silica gel 120 g (eluent: petroleum ether ethyl acetate 100%, 70:30) to give the title compound (2.5 g, 54%) as a colorless oil.

### Step 2: 1-tert-butyl 3,5-dimethyl 4-aminopyrazole-1,3,5-tricarboxylate

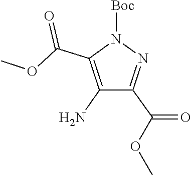 [SMILES: COC(=O)c1nn(C(=O)OC(C)(C)C)c(C(=O)OC)c1N]

The title compound was synthesized by the same method as described in Example 8, step 2 except 1-tert-butyl 3,5-dimethyl 4-nitropyrazole-1,3,5-tricarboxylate (1.70 g, 5.16 mmol) was used. The title compound (1.43 g, 86%) was obtained as an off-white solid.

### Step 3: 1-tert-butyl 3-methyl 7-oxo-6H-pyrazolo[4,3-d]pyrimidine-1,3-dicarboxylate

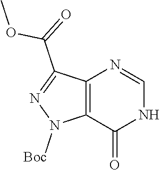 [SMILES: COC(=O)c1nn(C(=O)OC(C)(C)C)c2c(=O)[nH]cnc12]

To a stirred solution of 1-tert-butyl 3,5-dimethyl 4-aminopyrazole-1,3,5-tricarboxylate (2.00 g, 6.68 mmol, 1.00 equiv) in MeOH (40 mL) was added formamidine acetate (0.83 g, 8.02 mmol, 1.20 equiv) at room temperature. After stirring for 12 h at 105° C., the reaction mixture was cooled to room temperature and concentrated under reduced pressure. The residue was purified by CombiFlash with the following conditions, MeOH in water, 10% to 100% gradient in 30 min; detector, UV 254/220 nm.) to afford the title compound (1.2 g, 61%) as an off-white solid.

### Step 4: 1-tert-butyl 3-methyl 7-chloropyrazolo[4,3-d]pyrimidine-1,3-dicarboxylate

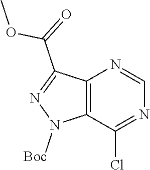 [SMILES: COC(=O)c1nn(C(=O)OC(C)(C)C)c2c(Cl)ncnc12]

To a solution of 1-tert-butyl 3-methyl 7-oxo-6H-pyrazolo[4,3-d]pyrimidine-1,3-dicarboxylate (1.20 g, 4.08 mmol, 1.00 equiv) in SO₂Cl₂ (20 mL) was added DMF (0.10 mL) at room temperature. After stirring for 2 h at 80° C., the reaction mixture was cooled to room temperature and concentrated under reduced pressure. The residue was purified CombiFlash with the following conditions, ACN in water, 10% to 50% gradient in 30 min; detector, UV 254/220 nm.) to afford the title compound (565 mg, 44%) as an off-white solid.

### Step 5: 1-tert-butyl 3-methyl 7-([[4-(diethoxyphosphoryl)phenyl]methyl]amino)pyrazolo[4,3-d]pyrimidine-1,3-dicarboxylate

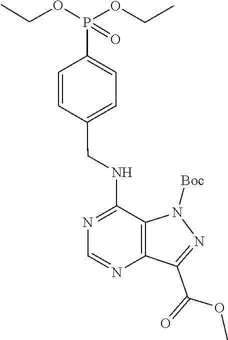 [SMILES: CCOP(=O)(OCC)c1ccc(CNc2ncnc3c(C(=O)OC)nn(C(=O)OC(C)(C)C)c23)cc1]

The title compound was synthesized by the same method as described in Example 1, step 2 except 1-tert-butyl 3-methyl 7-chloropyrazolo[4,3-d]pyrimidine-1,3-dicarboxylate (100 mg, 0.32 mmol) and diethyl 4-(aminomethyl)phenylphosphonate (93 mg, 0.38 mmol) were used. The title compound (160 mg, 87%) was obtained as a white solid.

### Step 6: tert-butyl 7-((4-(ethoxy(hydroxy)phosphoryl)benzyl)amino)-3-(methyl-carbamoyl)-1H-pyrazolo[4,3-d]pyrimidine-1-carboxylate

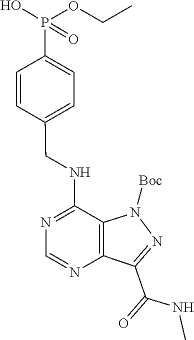 [SMILES: CCOP(=O)(O)c1ccc(CNc2ncnc3c(C(=O)NC)nn(C(=O)OC(C)(C)C)c23)cc1]

The title compound was synthesized by the same method as described in Example 2, step 2 except 1-tert-butyl 3-methyl 7-([[4-(diethoxyphosphoryl)phenyl]-methyl]amino)-pyrazolo[4,3-d]pyrimidine-1,3-dicarboxylate (160 mg, 0.31 mmol) was used. The title compound (140 mg, 83%) as a white solid was obtained.

### Step 7: ethoxy(4-([[3-(methylcarbamoyl)-1H-pyrazolo[4,3-d]pyrimidin-7-yl]amino]methyl)-phenyl)phosphinic acid

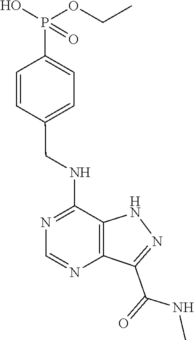 [SMILES: CCOP(=O)(O)c1ccc(CNc2ncnc3c(C(=O)NC)n[nH]c23)cc1]

A solution of 4-([[1-(tert-butoxycarbonyl)-3-(methylcarbamoyl)pyrazolo[4,3-d]pyrimidin-7-yl]amino]methyl)phenyl(ethoxy)phosphinic acid (110 mg, 0.22 mmol, 1.00 equiv) in 4 mL HCl (gas, 4 M in 1,4-dioxane) was stirred for 4 h at 50° C. was After cooling down to room temperature, the reaction mixture was concentrated under reduced pressure. The residue was purified by CombiFlash with the following conditions, ACN in water, 5% to 60% gradient in 30 min; detector, UV 254/220 nm.) to give the title compound (40 mg, 46%) as white solid. MS (ESI, pos. ion) m/z: 391.2 (M+1).

### Example 14

### Synthesis of 4-([[3-(methylcarbamoyl)-1H-pyrazolo[4,3-d]pyrimidin-7-yl]amino]methyl)-phenylphosphonic acid

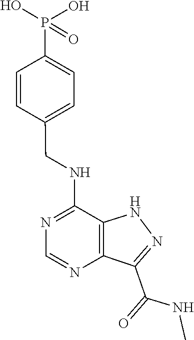 [SMILES: CNC(=O)c1n[nH]c2c(NCc3ccc(P(=O)(O)O)cc3)ncnc12]

The title compound was synthesized by the same method as described in Example 1, step 3 except ethoxy(4-([[3-(methylcarbamoyl)-1H-pyrazolo[4,3-d]pyrimidin-7-yl]amino]methyl)phenyl)phosphinic acid (40 mg, 0.10 mmol) was used. The crude product was purified by prep-HPLC with the following conditions (Column: Xselect CSH OBD Column 30*150 mm Sum, n; Mobile Phase A: Water (0.1% FA), Mobile Phase B: ACN; Flow rate: 60 mL/min; Gradient: 2% B to 13% B in 8 min, 13% B; Wave Length: 254220 nm;) to afford the title compound (13.2 mg, 35%) as a white solid. MS (ESI, pos. ion) m/z: 363.2 (M+1). ¹H NMR (300 MHz, DMSO-d₆ and D20, ppm) δ 8.33 (s, 1H), 7.61 (m, 2H), 7.39 (m, 2H), 4.78 (s, 2H), 2.91 (s, 3H).

### Example 15

### Synthesis of 4-[([3-carbamoyl-1H-pyrazolo[4,3-d]pyrimidin-7-yl]amino)methyl]-phenylphosphonic acid

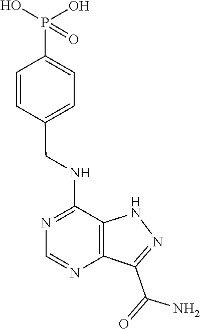 [SMILES: NC(=O)c1n[nH]c2c(NCc3ccc(P(=O)(O)O)cc3)ncnc12]

### Step 1: tert-butyl 3-carbamoyl-7-([[4-(diethoxyphosphoryl)phenyl]methyl]amino)pyrazolo-[4,3-d]pyrimidine-1-carboxylate

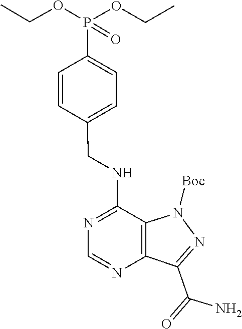 [SMILES: CCOP(=O)(OCC)c1ccc(CNc2ncnc3c(C(N)=O)nn(C(=O)OC(C)(C)C)c23)cc1]

The title compound was synthesized by the same method as described in Example 2, step 2 except 1-tert-butyl 3-methyl 7-([[4-(diethoxyphosphoryl)phenyl]-methyl]amino)pyrazolo[4,3-d]pyrimidine-1,3-dicarboxylate (140 mg, 0.27 mmol) and concentrated ammonia (10 mL) was used. The title compound (50 mg, 37%) was obtained as a white solid.

### Step 2: 4-[([3-carbamoyl-1H-pyrazolo[4,3-d]pyrimidin-7-yl]amino)methyl]phenyl-phosphonic acid

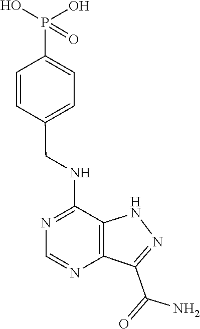 [SMILES: NC(=O)c1n[nH]c2c(NCc3ccc(P(=O)(O)O)cc3)ncnc12]

The title compound was synthesized by the same method as described in Example 1, step 3 except tert-butyl 3-carbamoyl-7-([[4-(diethoxyphosphoryl)phenyl]-methyl]amino)pyrazolo[4,3-d]pyrimidine-1-carboxylate (20 mg, 0.04 mmol) was used. The crude product was purified by prep-HPLC with the following conditions (Column: XSelect CSH Prep C18 OBD Column, 19*250 mm, 5 μm; Mobile Phase A: Water (0.1% FA), Mobile Phase B: ACN; Flow rate: 25 mL/min; Gradient: 2% B to 16% B in 7 min, 16% B; Wave Length: 254/220 nm;) to give the title compound (1.9 mg, 5%) as a white solid. MS (ESI, pos. ion) m/z: 348.9 (M+1). ¹H NMR (300 MHz, DMSO-d₆ and a drop of HCl (aq, 1 M), ppm) δ 11.07 (t, J=6.0 Hz, 1H), 8.62 (s, 1H), 8.30 (s, 1H), 8.16 (d, J=13.9 Hz, 1H), 7.92 (s, 1H), 7.65 (m, 2H), 7.51 (m, 2H), 5.00 (s, 2H).

### Example 16

### Synthesis of 4-[([7-acetamidothieno[3,2-d]pyrimidin-4-yl]amino)methyl]phenyl-phosphonic acid

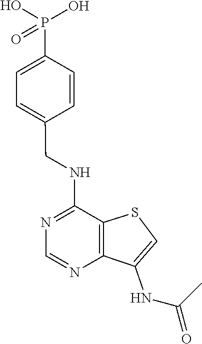 [SMILES: CC(=O)Nc1csc2c(NCc3ccc(P(=O)(O)O)cc3)ncnc12]

### Step 1: 7-nitrothieno[3,2-d]pyrimidin-4-ol

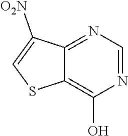 [SMILES: O=[N+]([O-])c1csc2c(O)ncnc12]

Into a 100-mL round-bottom flask, was placed H₂SO₄ (12.70 equiv) and the solution was stirred for 30 min at 0° C. 4-Chlorothieno[3,2-d]pyrimidine (1 equiv.) was added and the reaction mixture stirred for 30 min at 0° C. HNO₃ (7.5 equiv) was added and stirring was continued for 30 min at 0° C. The resulting mixture was allowed to warm to room temperature and stirred for 2 h. The reaction mixture was basified to pH 7 with saturated aqueous sodium bicarbonate. The precipitate was collected by filtration, washed with water and dried in vacuo to afford the title compound (5 g, 83%) was obtained as a white solid.

### Step 2: 4-chloro-7-nitrothieno[3,2-d]pyrimidine

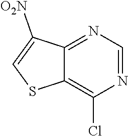 [SMILES: O=[N+]([O-])c1csc2c(Cl)ncnc12]

To a stirred solution of 7-nitrothieno[3,2-d]pyrimidin-4-ol (4.60 g, 23.33 mmol, 1.00 equiv) in thionyl chloride (120 mL) was added DMF (0.50 mL) at room temperature. After stirring for 2 h at 80° C., the reaction mixture was cooled to room temperature and concentrated under reduced pressure. The residue was purified by Combiflash with the following conditions, ACN in water, 10% to 40% gradient in 20 min; detector, UV 254/220 nm.) to give the title compound (3.26 g, 64%) as a white solid.

### Step 3: 4-chlorothieno[3,2-d]pyrimidin-7-amine

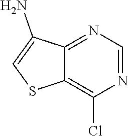 [SMILES: Nc1csc2c(Cl)ncnc12]

To a stirred solution of 4-chloro-7-nitrothieno[3,2-d]pyrimidine (3.26 g, 15.12 mmol, 1.00 equiv) in EtOH (180 mL) and H₂O (30 mL) were added Fe (8.44 g, 0.15 mmol, 10.00 equiv) and NH₄Cl (8.09 g, 0.15 mmol, 10.00 equiv) at room temperature under e nitrogen. The resulting mixture was stirred for 2 h at 80° C. After cooling down to room temperature, the reaction mixture was filtered through a Celite and the filter cake was washed with EtOH. The filtrate was concentrated under reduced pressure. The residue was purified by column chromatography on silica gel 120 g (eluent: petroleum ether ethyl acetate 100%, 64:36) to give the title compound (1.8 g, 63%) as a yellow solid.

### Step 4: N-[4-chlorothieno[32-d]pyrimidin-7-yl]acetamide

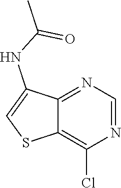

To a stirred solution of 4-chlorothieno[3,2-d]pyrimidin-7-amine (300 mg, 1.62 mmol, 1.00 equiv) in DCM (8 mL) were added DIEA (418 mg, 3.23 mmol, 2.00 equiv) and acetyl chloride (254 mg, 3.23 mmol, 2.00 equiv) at 0° C. After stirring for 1 h at 0° C., the mixture was concentrated under reduced pressure. The residue was purified by column chromatography on silica gel 40 g (eluent: petroleum ether ethyl acetate 100%, 82:18) to give the title compound (310 mg, 83%) as a white solid.

### Step 5: diethyl 4-[([7-acetamidothieno[3,2-d]pyrimidin-4-yl]amino)methyl]phenyl-phosphonate

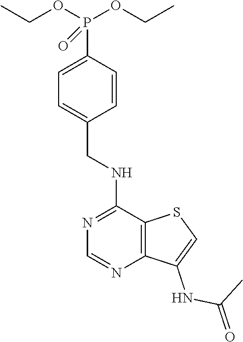 [SMILES: CCOP(=O)(OCC)c1ccc(CNc2ncnc3c(NC(C)=O)csc23)cc1]

The title compound was synthesized by the same method as described in Example 1, step 2 except N-[4-chlorothieno[3,2-d]pyrimidin-7-yl]acetamide (170 mg, 0.75 mmol) was used. The title compound (300 mg, 91%) was obtained as a brown oil.

### Step 6: 4-[([7-acetamidothieno[3,2-d]pyrimidin-4-yl]amino)methyl]phenylphosphonic acid

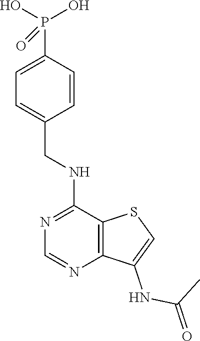 [SMILES: CC(=O)Nc1csc2c(NCc3ccc(P(=O)(O)O)cc3)ncnc12]

The title compound was synthesized by the same method as described in Example 1, step 3 except diethyl4-[([7-acetamidothieno[3,2-d]pyrimidin-4-yl]amino)-methyl]phenylphosphonate (150 mg, 0.35 mmol) was used. The crude product was purified by prep-HPLC with the following conditions (Column: Xselect CSH OBD Column 30*150 mm 5 um, n; Mobile Phase A: Water (0.1% FA), Mobile Phase B: ACN; Flow rate: 60 mL/min; Gradient: 2% B to 14% B in 7 min, 14% B; Wave Length: 254/220 nm;) to give the title compound (35.8 mg, 27%) as a white solid. MS (ESI, pos. ion) m/z: 379.0 (M+1). ¹H NMR (300 MHz, DMSO-d₆, ppm) δ 10.20 (s, 1H), 8.57 (t, J=5.9 Hz, 1H), 8.48 (s, 1H), 8.23 (s, 1H), 7.62 (d, J=12.6, 7.8 Hz, 2H), 7.37 (d, J=8.2, 3.2 Hz, 2H), 4.76 (d, J=5.9 Hz, 2H), 2.17 (s, 3H).

### Example 17

### Synthesis of 4-([thieno[3,2-d]pyrimidin-4-ylamino]methyl)phenylphosphonic acid

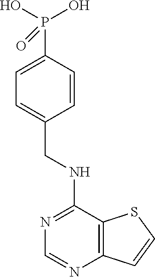 [SMILES: O=P(O)(O)c1ccc(CNc2ncnc3ccsc23)cc1]

### Step 1: diethyl 4-([thieno[3,2-d]pyrimidin-4-ylamino]methyl)phenylphosphonate

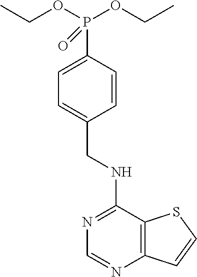 [SMILES: CCOP(=O)(OCC)c1ccc(CNc2ncnc3ccsc23)cc1]

The title compound was synthesized by the same method as described in Example 1, step 2 except 4-chlorothieno[3,2-d]pyrimidine (130 mg, 0.76 mmol) was used. The title compound (250 mg, 87%) was obtained as a white solid.

### Step 2: 4-([thieno[3,2-d]pyrimidin-4-ylamino]methyl)phenylphosphonic acid

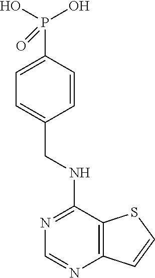 [SMILES: O=P(O)(O)c1ccc(CNc2ncnc3ccsc23)cc1]

The title compound was synthesized by the same method as described in Example 1, step 3 except diethyl 4-([thieno[3,2-d]pyrimidin-4-ylamino]methyl)phenyl-phosphonate (125 mg, 0.33 mmol) was used. The crude product was purified by prep-HPLC with the following conditions (Column: Xselect CSH OBD Column 30*150 mm 5 um, n; Mobile Phase A: Water (0.1% FA), Mobile Phase B: ACN; Flow rate: 60 mL/min; Gradient: 2% B to 10% B in 7 min, 10% B; Wave Length: 254/220 nm;) to give the title compound (53.9 mg, 50%) as a white solid. MS (ESI, pos. ion) m/z: 322.2 (M+1). ¹H NMR (300 MHz, DMSO-d₆ and a drop of HCl (aq, 1M), ppm) δ 10.59 (t, J=6.0 Hz, 1H), 8.86 (s, 1H), 8.49 (d, J=5.4 Hz, 1H), 7.71-7.52 (m, 3H), 7.46 (d, J=8.1, 3.4 Hz, 2H), 4.90 (d, J=5.9 Hz, 2H).

### Example 18

### Synthesis of 4-[([5-isopropyl-1H-pyrazolo[4,3-d]pyrimidin-7-yl]amino)methyl]phenyl-phosphonic acid formic acid salt

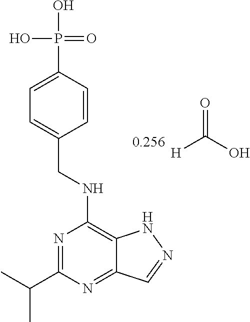 [SMILES: CC(C)c1nc(NCc2ccc(P(=O)(O)O)cc2)c2[nH]ncc2n1 ; O=CO]

### Step 1: diethyl 4-[([5-isopropyl-1H-pyrazolo[4,3-d]pyrimidin-7-yl]amino)methyl]phenyl-phosphonate

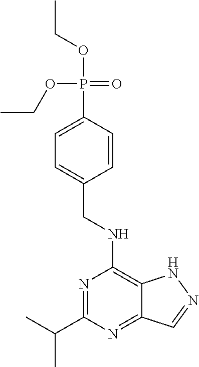 [SMILES: CCOP(=O)(OCC)c1ccc(CNc2nc(C(C)C)nc3cn[nH]c23)cc1]

To a solution of 7-chloro-5-isopropyl-1H-pyrazolo[4,3-d]pyrimidine (50 mg, 0.25 mmol, 1.0 equivalent) in ethanol (5 ml) were added diethyl 4-(aminomethyl)phenylphosphonate (74 mg, 0.30 mmol, 1.2 equivalents) and TEA (51 mg, 0.50 mmol, 2.0 equivalents). After stirring overnight at 80° C., the reaction mixture was concentrated under reduced pressure. The residue was purified by silica gel column chromatography, eluted with EA/PE (3:1) to afford the title compound (80 mg, 77%) as a light yellow oil.

### Step 2: 4-[([5-isopropyl-1H-pyrazolo[4,3-d]pyrimidin-7-yl]amino)methyl]phenylphosphonic acid formic acid salt

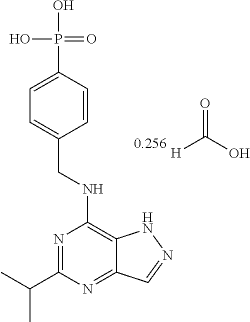 [SMILES: CC(C)c1nc(NCc2ccc(P(=O)(O)O)cc2)c2[nH]ncc2n1 ; O=CO]

To a solution of diethyl 4-[([5-isopropyl-1H-pyrazolo[4,3-d]pyrimidin-7-yl]amino)-methyl]phenylphosphonate (80 mg, 0.19 mmol, 1.0 equiv) in DCM (5 mL) was added bromotrimethylsilane (2 mL) at room temperature. After stirring at room temperature for 48 h, the reaction mixture was concentrated under reduce pressure. The residue was purified by prep-HPLC with the following conditions: Column: Xselect CSH C18 OBD Column 30*150 mm 5 μm; Mobile Phase A: Water (0.1% FA), Mobile Phase B: ACN; Flow rate: 60 mL/min; Gradient: 3% B to 16% B in 9 min, 16% B; Wave Length: 254/220 nm). The collected fractions were combined and concentrated to give the title compound (21.5 mg, 27%) as an off-white solid. MS (ESI, pos. ion) m/z: 348.3 (M+1). ¹H-NMR: (400 MHz, DMSO-d& ppm) δ 8.14 (s, 0.256H), 8.04 (s, 1H), 7.65-7.55 (m, 2H), 7.43-7.37 (m, 2H), 4.76 (s, 2H), 2.96-2.90 (m, 1H), 1.21 (d, J=6.8 Hz, 6H).

### BIOLOGICAL EXAMPLES

### Example 1

### Measurement of pNP-TMP Hydrolysis by ENPP1

p-Nitrophenyl thymidine 5′-monophosphate (pNP-TMP) is a synthesized substrate for ENPP1. The ENPP1 enzyme activity assay with pNP-TMP substrate was conducted as follows:

First, in a 60 μl reaction, 7.5 ng purified ENPP1 was mixed with compounds of Formula (I) (test compound) ranging from 13.7 pM to 10 μM. Incubation of ENPP1 with compounds was set at 25° C. for 10 min. Reactions with DMSO only (with ENPP1 but no compound) gave the fastest reaction (MAX Activity). For each compound dilution, wells with assay buffer (50 mM Tris-HCl, pH8.8, 250 mM NaCl, 0.1 mg/ml BSA, 1% DMSO) only but no ENPP1 were included as controls for subtraction of test compound derived absorbance at 405 nm.

Second, after the 10 minutes ENPP1 and test compound incubation the assay was initiated by transferring 50 μl of the above mentioned ENPP1/test compound reaction into 50 μl of 1 mM pNP-TMP in assay buffer results in a 100 μl total reaction in clear bottom 96 well plates. Absorbance at 405 nm was recorded immediately in kinetic mode by PerkinElmer 2300 Enspire multimode plate reader.

For each inhibitor, the specific ENPP1 activity was calculated using the following equation: ENPP1 activity (pmol/min/μg)=Adjusted Vmax (OD405 nm/min)×conversion factor (pmol/OD405 nm)/amount of enzyme (μg)

Adjusted Vmax=V₀×(Km+(S))/(S). In this assay, Km=232 μM, (S)=500 μM. Adjusted Vmax=1.464×V₀.

V₀=(OD405 nm with ENPP1−OD405 nm ENPP1 blank)/minutes. OD405 nm was plotted, with blank subtracted, against time (minutes), the initial linear rate is V₀. blank subtracted, against time (minutes), the initial linear rate is V₀.

The conversion factor (pmol/OD405 nm), was determined by plotting the amount of standard, 4-Nitrophenol (Sigma-Aldrich, Catalog #241326), against absorbance at 405 nm. The slope is the conversion factor. The percent ENPP1 activity for each sample was calculated using the following equation:

% enzyme activity=sample enzyme activity/MAX Activity×100%.

To determine the IC₅₀ for each compound, compound concentration values and percent enzyme activity values were inserted into GraphPad Prism (GraphPad Prism version 7.0 for Windows, GraphPad Software, La Jolla California USA, www.graphpad.com), and Prism's Transform analysis was used to convert the x-axis values (compound concentration) to logarithms. A sigmoidal variable slope nonlinear regression analysis was performed using the following equation: Y=Bottom+(Top−Bottom)/(1+10\hat{ }((Log IC₅₀−X)*HillSlope)).

Kᵢ values for each compound were calculated from the observed IC₅₀ from GraphPad analysis using the Cheng-Prusoff equation: Kᵢ=IC₅₀/(1+(S)/K_{M}). (S) here is 500 μM and K_{M} is determined to be 232 μM.

Ki for a representative compound of Formula (I) in Compound Table 1 above is provided in Table 2 below:

TABLE 2
Ki (pNP-TMP)

| Group A | Group B | Group C | Group D |
|---|---|---|---|
| (<0.10 μM) | (<1 μM) | (<10 μM) | (>10 μM) |
| 1, 12, 15, 16 | 3, 4, 6, 9, 14, 17, 18 | 11 | 2, 7,10 |

### Example 2

### Measurement of 2′3′-cGAMP hydrolysis by ENPP1

ENPP1 catalyzes the hydrolysis of 2′3′-cGAMP into 5′-AMP and 5′-GMP, and hence the ENPP1 enzyme activity with 2′3′-cGAMP as substrate is monitored by measurement of the product 5′-AMP. The AMP-Glo assay kit from Promega (catalog number V5012) is used for measurement of 5′-AMP production.

First, an ENPP1 and test compound incubation is set up in assay buffer (50 mM Tris-HCl, pH8.8, 250 mM NaCl, 0.1 mg/ml BSA, 1% DMSO) with following conditions: ENPP1 concentration: 1.25 nM; test compound concentration ranging from 68 pM to 20 μM. This incubation is carried out at 25° C. for 10 min.

Second, after the 10 minute ENPP1 and test compound incubation, prepare on a separate plate, 15 μl of the substrate 2′3′-cGAMP at 200 pM in assay buffer. Then, 15 μl of the ENPP1/Compound incubation is transferred to the 200 μM 2′3′-cGAMP solution to initiate the reaction. The 30 μl mixture is incubated for 30 min at 25° C. In all these assays a DMSO control without compound is included which gave the maximum 5′-AMP production (MAX RLU). After 30 min the reaction is stopped by heating at 90° C. for 3 min.

Third, the Promega AMP-Glo kit is used to detect 5′-AMP production as a measurement of ENPP1 enzyme activity. To do this 10 μl of the above mentioned 30 μl total reaction per sample is transferred into 384 well white solid assay plate for measurement of 5′-AMP production. For each well, 10 μl of AMP-Glo Reagent I is added, mixed well, and incubated for 1 hour at 25° C. At this time AMP detection solution is prepared and 20 μl is added per well, and the resulting solution is incubated for 1 hr at 25° C. Duplicates are run for each inhibitor concentration. Luminescence signal (relative luminescence units, RLU) is recorded using a PerkinElmer 2300 Enspire multimode plate reader.

The % inhibition is calculated using the following equation: % inhibition=(MAX RLU−sample RLU)/MAX RLU×100%.

IC₅₀ values of compounds are determined by loading compound concentration data and percent inhibition values into GraphPad Prism (GraphPad Prism version 7.0 for Windows, GraphPad Software, La Jolla California USA, www.graphpad.com) and conducted a Sigmoidal variable slope nonlinear regression fitting.

Kᵢ values for each compound are calculated from the observed IC₅₀ from GraphPad analysis using the Cheng-Prusoff equation: Kᵢ=IC₅₀/(1+(S)/K_{M}). (S) here is 100 μM and K_{M} is 32 μM.

### FORMULATION EXAMPLES

The following are representative pharmaceutical formulations containing a compound of the present disclosure.

### Tablet Formulation

The following ingredients are mixed intimately and pressed into single scored tablets.

| | Quantity per tablet |
|---|---|
| Ingredient | mg |
| compound of this disclosure | 400 |
| cornstarch | 50 |
| croscarmellose sodium | 25 |
| lactose | 120 |
| magnesium stearate | 5 |

### Capsule Formulation

The following ingredients are mixed intimately and loaded into a hard-shell gelatin capsule.

| | Quantity per capsule |
|---|---|
| Ingredient | mg |
| compound of this disclosure | 200 |
| lactose spray dried | 148 |
| magnesium stearate | 2 |

### Injectable Formulation

Compound of the disclosure (e.g., compound 1) in 2% HPMC, 1% Tween 80 in DI water, pH 2.2 with MSA, q.s. to at least 20 mg/mL.

### Inhalation Composition

To prepare a pharmaceutical composition for inhalation delivery, 20 mg of a compound disclosed herein is mixed with 50 mg of anhydrous citric acid and 100 mL of 0.9% sodium chloride solution. The mixture is incorporated into an inhalation delivery unit, such as a nebulizer, which is suitable for inhalation administration.

### Topical Gel Composition

To prepare a pharmaceutical topical gel composition, 100 mg of a compound disclosed herein is mixed with 1.75 g of hydroxypropyl cellulose, 10 mL of propylene glycol, 10 mL of isopropyl myristate and 100 mL of purified alcohol USP. The resulting gel mixture is then incorporated into containers, such as tubes, which are suitable for topical administration.

### Ophthalmic Solution Composition

To prepare a pharmaceutical ophthalmic solution composition, 100 mg of a compound disclosed herein is mixed with 0.9 g of NaCl in 100 mL of purified water and filtered using a 0.2 micron filter. The resulting isotonic solution is then incorporated into ophthalmic delivery units, such as eye drop containers, which are suitable for ophthalmic administration.

### Nasal Spray Solution

To prepare a pharmaceutical nasal spray solution, 10 g of a compound disclosed herein is mixed with 30 mL of a 0.05M phosphate buffer solution (pH 4.4). The solution is placed in a nasal administrator designed to deliver 100 ul of spray for each application.

What is claimed:

1. A compound of Formula (I): 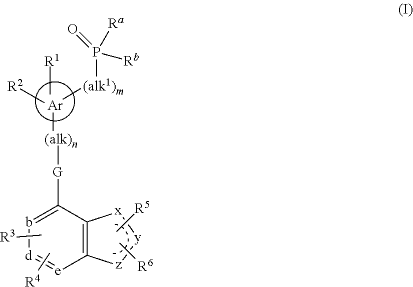
wherein: [symbol]
dashed line is a bond between x and y or y and z;
b, d, and e are CH; or one or two of b, d, and e are N and remaining of b, d, and e are CH;
one of x and z is NH, O, or S and the other of x and z is CH or N; and y is CH or N; provided that, at least one of y and the x or z that is CH or N, is CH;
G is a bond, NR (where R is hydrogen or alkyl), O, S, or SO₂;
alk is alkylene optionally substituted with one, two, or three halo, or alkynylene, provided that when alk is alkynylene G is a bond and n is 1;
alk¹ is alkylene optionally substituted with one, two, or three halo;
m and n are independently 0 or 1; provided that at least one of m and n is 1;
Ar is aryl, heteroaryl, or heterocyclyl;
R^{a} and R^{b} are independently selected from hydroxy, alkoxy, —Oaryl (where aryl is optionally substituted with one to three substituents independently selected from alkyl, alkenyl, alkoxy, halo, haloalkyl, amino, alkylamino, dialkylamino, cyano, and nitro), —O—(CH₂)OCOR^{c} (where R^{c} is alkyl), —O—(CH₂)OCOOR^{c1} (where R^{c1} is alkyl), —O-(alk²)OR^{d} (where alk² is alkylene and R^{d} is alkyl), —S—(CH₂)₂SCOR^{c}(where R^{c} is alkyl), and —NR^{g}—(CHR^{h})OCOR^{f} (where R^{h} is hydrogen, alkyl, hydroxymethyl, thiomethyl, methylthiomethyl, amidinopropyl, indol-3-ylmethyl, indol-4-ylmethyl, carboxymethyl, carboxyethyl, aminocarbonylmethyl, aminocarbonylethyl, phenyl or phenylalkyl (wherein phenyl, either alone or as part of phenylalkyl, is optionally substituted with one to three substituents independently selected from alkyl, alkoxy, halo, hydroxy, cyano, and nitro), R^{f} is alkyl or benzyl and R^{g} is hydrogen or R^{g} together with R^{h} forms —(CH₂)₃—); or
R^{a} and R^{b} together with the phosphorus atom to which they are attached form a ring of formula (a): 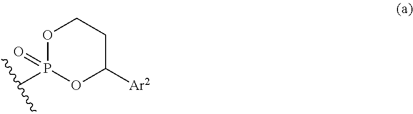
wherein Ar² is phenyl or six membered heteroaryl wherein the phenyl and six membered heteroaryl aryl optionally substituted with one to three halo;
R¹ and R² are independently absent, alkyl, alkoxy, halo, haloalkyl, haloalkoxy, or cyano;
R³ and R⁴ are independently absent, alkyl, alkoxy, hydroxy, halo, haloalkyl, haloalkoxy, alkylsulfonyl, hydroxyalkyl, alkoxyalkyl, hydroxyalkoxy, alkoxyalkoxy, hydroxyalkylamino, alkoxyalkylamino, amino, aminoalkyl, aminoalkoxy, aminoalkylamino, heterocyclyl, heterocyclyloxy, heterocyclylamino (wherein heterocyclyl, either alone or part of heterocyclyloxy, and heterocyclylamino is optionally substituted with Rⁱ, R^{j}, and/or R^{k} independently selected from alkyl, halo, hydroxy, alkoxy, hydroxyalkyl, alkoxyalkyl, and aminoalkyl), heterocyclylalkyl, heterocyclylalkyloxy, heterocyclylalkylamino (wherein the heterocyclyl ring in heterocyclylalkyl, heterocyclylalkyloxy, and heterocyclylalkylamino is optionally substituted with one, two, or three substituents independently selected from alkyl, halo, hydroxy, alkoxy, hydroxyalkyl, alkoxyalkyl, and aminoalkyl), cycloalkyloxy, phenyl, heteroaryl, phenylalkenyl, heteroarylalkenyl, phenyloxy, and heteroaryloxy (where phenyl, by itself or as part of phenylalkenyl and phenyloxy and heteroaryl, by itself or as part of heteroarylalkenyl and heteroaryloxy are optionally substituted with one, two, or three substituents independently selected from alkyl, hydroxy, alkoxy, halo, haloalkyl, haloalkoxy, and cyano); provided that when two of b, d, and e are N, then at least one of R³ and R⁴ is absent;
R⁵ is absent, alkyl, hydroxy, alkoxy, alkoxycarbonyl, halo, acylamino, hydroxyalkylamino, alkoxyalkylamino, cyano, aminocarbonyl, alkylaminocarbonyl, dialkylaminocarbonyl, aminoalkyloxycarbonyl, alkoxyalkyloxycarbonyl, or hydroxyalkylaminocarbonyl; and
R⁶ is absent or alkyl; provided that one of R³ and R⁶ is absent when two of x, y, and z are other than CH; or
a pharmaceutically acceptable salt thereof.

2. The compound of claim 1, or a pharmaceutically acceptable salt thereof, wherein
alk is alkylene optionally substituted with one, two, or three halo;
R³ and R⁴ are independently absent, alkyl, alkoxy, hydroxy, halo, haloalkyl, haloalkoxy, alkylsulfonyl, hydroxyalkyl, alkoxyalkyl, hydroxyalkoxy, alkoxyalkoxy, hydroxyalkylamino, alkoxyalkylamino, amino, aminoalkyl, aminoalkoxy, aminoalkylamino, heterocyclyl, heterocyclyloxy, heterocyclylamino (wherein heterocyclyl, either alone or part of heterocyclyloxy, and heterocyclylamino is optionally substituted with Rⁱ, R^{j}, and/or R^{k} independently selected from alkyl, halo, hydroxy, alkoxy, hydroxyalkyl, alkoxyalkyl, and aminoalkyl), heterocyclylalkyl, heterocyclylalkyloxy, heterocyclylalkylamino (wherein the heterocyclyl ring in heterocyclylalkyl, heterocyclylalkyloxy, and heterocyclylalkylamino is optionally substituted with one, two, or three substituents independently selected from alkyl, halo, hydroxy, alkoxy, hydroxyalkyl, alkoxyalkyl, and aminoalkyl), cycloalkyloxy, phenyloxy, and heteroaryloxy (where phenyl in phenyloxy and heteroaryl in heteroaryloxy are optionally substituted with one, two, or three substituents independently selected from alkyl, hydroxy, alkoxy, halo, haloalkyl, haloalkoxy, and cyano); provided that when two of b, d, and e are N, then at least one of R³ and R⁴ is absent; and
R⁵ is absent, alkyl, hydroxy, halo, acylamino, hydroxyalkylamino, alkoxyalkylamino, cyano, aminocarbonyl, alkylaminocarbonyl, or dialkylaminocarbonyl.

3. The compound of claim 2, or a pharmaceutically acceptable salt thereof, wherein:
x is NH, O, or S;
y and z are independently CH or N; provided that at least one of y and z is CH; and
G is NR (where R is hydrogen or alkyl), O, or S.

4. The compound of claim 3, or pharmaceutically acceptable salt thereof, wherein
G is NR (where R is hydrogen or alkyl) or O;

5. The compound of any one of claims 2, 3, or 4, or a pharmaceutically acceptable salt thereof, having a structure of formula (Ia) or (Ib):

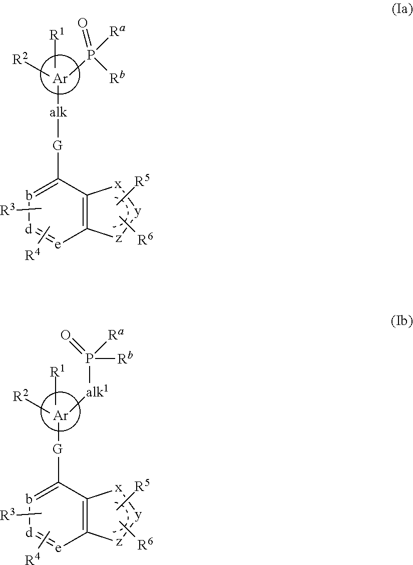

6. The compound of claim 5, or a pharmaceutically acceptable salt thereof, having a structure of formula (Ia).

7. The compound of claim 5, or a pharmaceutically acceptable salt thereof, having a structure of formula (Ib).

8. The compound of any one of claims 2, 3 or 4, or a pharmaceutically acceptable salt thereof, having a structure of formula (Ic) or (Id): 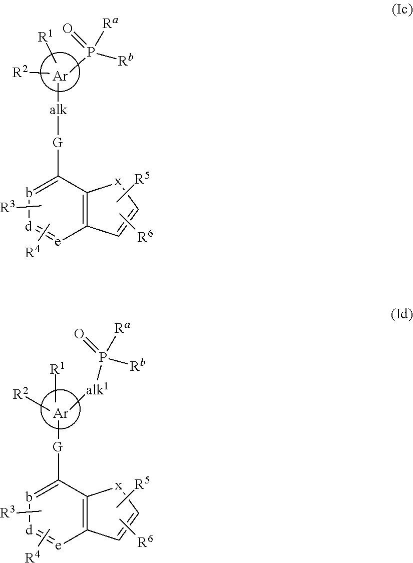
wherein x is NH, O, or S.

9. The compound of claim 8, or a pharmaceutically acceptable salt thereof, having a structure of formula (Ic).

10. The compound of claim 8, or a pharmaceutically acceptable salt thereof, having a structure of formula (Id).

11. The compound of any one of claims 8 to 10, or a pharmaceutically acceptable salt thereof, wherein x is NH.

12. The compound of any one of claims 8 to 10, or a pharmaceutically acceptable salt thereof, wherein x is O.

13. The compound of any one of claims 8 to 10, or a pharmaceutically acceptable salt thereof, wherein x is S.

14. The compound of claim 11, or a pharmaceutically acceptable salt thereof, wherein R⁶ is either attached to the nitrogen of NH or the carbon of the 5-membering ring that is adjacent to x, and R⁵ when present is attached to carbon of the 5-membered ring that is adjacent to a bridgehead carbon.

15. The compound of claim 12 or 13, or a pharmaceutically acceptable salt thereof, wherein R⁶ is attached to the carbon of the 5-membered ring that is adjacent to x and R⁵ when present is attached to carbon of the 5-membered ring that is adjacent to a bridgehead carbon.

16. The compound of any one of claims 2, 3 or 4, or a pharmaceutically acceptable salt thereof, having a structure of formula (Ie) or (If): 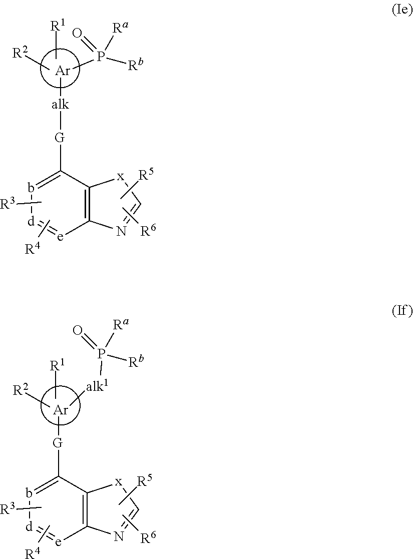
wherein x is NH, O, or S.

17. The compound of claim 16, or a pharmaceutically acceptable salt thereof, having a structure of formula (Ie).

18. The compound of claim 16, or a pharmaceutically acceptable salt thereof, having a structure of formula (If).

19. The compound of any one of claims 16 to 18, or a pharmaceutically acceptable salt thereof, wherein x is NH.

20. The compound of any one of claims 16 to 18, or a pharmaceutically acceptable salt thereof, wherein x is O.

21. The compound of any one of claims 16 to 18, or a pharmaceutically acceptable salt thereof, wherein x is S.

22. The compound of claim 19, or a pharmaceutically acceptable salt thereof, wherein R⁶ is attached to the nitrogen of NH and R³ is attached to carbon of the 5-membered ring that is adjacent to x.

23. The compound of claim 20 or 21, or a pharmaceutically acceptable salt thereof, wherein R⁶ is absent and R⁵ is attached to carbon of the 5-membered ring that is adjacent to x.

24. The compound of any one of claims 2, 3 or 4, or a pharmaceutically acceptable salt thereof, having a structure of formula (Ig) or (Ih): 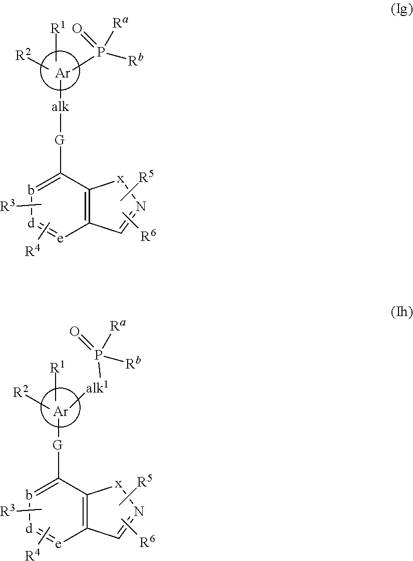
wherein x is NH, O, or S.

25. The compound of claim 24, or a pharmaceutically acceptable salt thereof, having a structure of formula (Ig).

26. The compound of claim 24, or a pharmaceutically acceptable salt thereof, having a structure of formula (Ih).

27. The compound of any one of claims 24 to 26, or a pharmaceutically acceptable salt thereof, wherein x is NH.

28. The compound of any one of claims 24 to 26, or a pharmaceutically acceptable salt thereof, wherein x is O.

29. The compound of any one of claims 24 to 26, or a pharmaceutically acceptable salt thereof, wherein x is S.

30. The compound of any one of claims 27 to 29, or a pharmaceutically acceptable salt thereof, wherein R⁶ is absent and R⁵ is attached to carbon of the 5-membered ring that is adjacent to a bridgehead carbon.

31. The compound of any one of claims 2 to 30, or a pharmaceutically acceptable salt thereof, wherein G is NR.

32. The compound of any one of claims 2 to 30, or a pharmaceutically acceptable salt thereof, wherein G is O.

33. The compound of any one of claims 2 to 32, or a pharmaceutically acceptable salt thereof, wherein b, d, and e are CH.

34. The compound of any one of claims 2 to 32, or a pharmaceutically acceptable salt thereof, wherein b is N and d, and e are CH.

35. The compound of any one of claims 2 to 32, or a pharmaceutically acceptable salt thereof, wherein d is N and b and e are CH.

36. The compound of any one of claims 2 to 32, or a pharmaceutically acceptable salt thereof, wherein e is N and b and d are CH.

37. The compound of any one of claims 2 to 32, or a pharmaceutically acceptable salt thereof, wherein b and e are N and d is CH.

38. The compound of any one of claims 2 to 37, or a pharmaceutically acceptable salt thereof, wherein —P(═O)(R^{a})(R^{b}) is —P(═O)(OH)₂.

39. The compound of any one of claims 2 to 38, or a pharmaceutically acceptable salt thereof, wherein Ar is phenyl.

40. The compound of any one of claims 2 to 38, or a pharmaceutically acceptable salt thereof, wherein Ar is phenyl and —P(═O)(R^{a})(R^{b}) is attached to carbon on the phenyl ring that is para to the carbon attaching the phenyl ring to remaining compound of Formula (I), (IA), (IB), and (Ia) to (Ih).

41. The compound of any one of claims 2 to 40, or a pharmaceutically acceptable salt thereof, wherein alk and alk¹ are independently methylene, ethylene, or propylene.

42. The compound of any one of claims 2 to 40, or a pharmaceutically acceptable salt thereof, wherein alk and alk¹ are methylene.

43. The compound of any one of claims 2 to 42, or a pharmaceutically acceptable salt thereof, wherein R⁶ is absent, methyl, or isopropyl.

44. The compound of any one of claims 2 to 43, or a pharmaceutically acceptable salt thereof, wherein R⁵ is absent, alkyl, hydroxy, halo, acylamino, alkoxyalkylamino, cyano, aminocarbonyl, alkylaminocarbonyl, or dialkylaminocarbonyl.

45. The compound of any one of claims 2 to 43, or a pharmaceutically acceptable salt thereof, wherein R³ is aminocarbonyl, methylaminocarbonyl, ethylaminocarbonyl, dimethylaminocarbonyl, or isopropylcarbonyl.

46. The compound of any one of claims 2 to 45, or a pharmaceutically acceptable salt thereof, wherein R¹ and R² are independently absent, methyl, ethyl, methoxy, fluoro, trifluoromethyl, trifluoromethoxy, or cyano.

47. The compound of claim 46, or a pharmaceutically acceptable salt thereof, wherein R¹ and R² are absent.

48. The compound of any one of claims 2 to 47, or a pharmaceutically acceptable salt thereof, wherein R³ and R⁴ are independently absent, alkyl, alkoxy, hydroxy, amino, halo, haloalkyl, or haloalkoxy.

49. A pharmaceutical composition comprising a compound any one of claims 1 to 48, and a pharmaceutically acceptable excipient.

50. A method of treating a disease or condition mediated by ENPP1 in a patient comprising administering to the patient a compound of any one of claims 1 to 48, or a pharmaceutical composition of claim 49 in a therapeutically effective amount.

51. The method of claim 50, wherein the disease or condition is a cancer, an inflammatory disease, a metabolic disease, or a viral disease.

52. The method of claim 51, wherein the disease or condition is a cancer.

53. The method of claim 52, wherein the disease or condition is a cancer wherein the cancer is hepatocellular carcinomas, glioblastomas, melanomas, testicular, pancreatic, thyroid or breast cancer.

54. The method of claim 52 or 53, wherein the compound of any one of claims 1 to 48 is administered in combination with another anticancer agent.
